(19) United States
(12) Patent Application Publication
(10) Pub. No.: US 2024/0199624 A1
(43) Pub. Date: Jun. 20, 2024

(54) COMPOUNDS, COMPOSITIONS, AND METHODS

(71) Applicant: CHDI Foundation, Inc., New York, NY (US)

(72) Inventors: Tasir Haque, Yardley, PA (US); Michel Maillard, Goleta, CA (US); Gareth Brace, Wantage (GB); George Ballantyne, Abingdon (GB); Elizabeth Frush, Pennington, NJ (US); Kim Hirst, Saffron Walden (GB); Katherine Jones, Norwich (GB); Emanuela Gancia, Royston (GB); Chris Lock, Cambridge (GB); Cole Clissold, Cambridge (GB)

(21) Appl. No.: 18/521,696

(22) Filed: Nov. 28, 2023

Related U.S. Application Data
(60) Provisional application No. 63/428,690, filed on Nov. 29, 2022.

(51) Int. Cl. C07D 487/04; A61K 9/02; A61K 9/20; A61K 9/48; A61K 47/12; A61K 47/14; A61K 47/26

(52) U.S. Cl. CPC C07D 487/04; A61K 9/02; A61K 9/2013; A61K 9/2018; A61K 9/2054; A61K 9/2059; A61K 9/4858; A61K 47/12; A61K 47/14; A61K 47/26

(57) ABSTRACT

The present disclosure relates generally to small molecule inhibitors of MutSβ protein, or a pharmaceutically acceptable salt, isotopically enriched analog, stereoisomer, mixture of stereoisomers, or prodrug thereof, methods of making and intermediates thereof, and methods of using thereof.

### CROSS-REFERENCE TO RELATED APPLICATIONS

This application claims the benefit under 35 U.S.C. § 119(e) of U.S. Provisional Application Ser. No. 63/428,690, filed Nov. 29, 2022, the contents of which are hereby incorporated by reference in its entirety.

### FIELD

The present disclosure relates generally to small molecule MutSβ modulators, and their use as therapeutic agents.

### BACKGROUND

Huntington's Disease (HD) is a neurodegenerative disease caused by a mutation in the huntingtin gene. Expansion of trinucleotide repeats in the huntingtin gene results in mutant huntingtin protein (mHtt), which leads to neuronal damage. The degradation of neurons in those suffering from HD can present as a wide variety of symptoms, including changes in mood and behavior, agitation, sensory disturbances, motor and cognitive difficulties, and memory loss, which can progress to inability to move or speak, dementia, and ultimately death.

Recent studies have suggested that the MutSβ complex, a DNA mismatch repair protein comprised of MSH2 and MSH3 proteins, has been linked to the progression of HD. It has been suggested that inhibition of MutSβ inhibits somatic expansion of trinucleotide repeats and thus has therapeutic potential for the treatment of HD.

### SUMMARY

Provided herein are compounds, or a pharmaceutically acceptable salt, isotopically enriched analog, stereoisomer, mixture of stereoisomers, or prodrug thereof, that are useful in treating and/or preventing diseases mediated, at least in part, by MutSβ.

In certain embodiments, provided are compounds of Formula I:

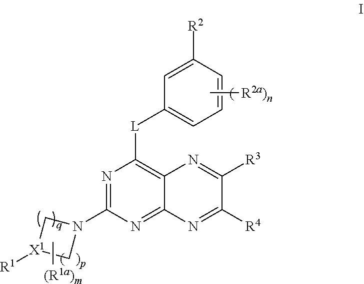

or a pharmaceutically acceptable salt, isotopically enriched analog, stereoisomer, mixture of stereoisomers, or prodrug thereof, wherein:
n is 0, 1, 2, 3, or 4;
m is 0, 1, 2, 3, 4, or 5;
p is 0, 1, 2, 3, 4, or 5;
q is 0, 1, 2, 3, or 4; provided that p+q is 2, 3, 4, or 5;
L is —NH—, —O—, —CH₂—, —NHCH₂—, —OCH₂—, or —CH₂CH₂—;
X¹ is CR⁵ or N;
R¹ is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R^{1a} and R^{2a} are each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R² is halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R³ and R⁴ are each independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R⁵ is hydrogen, C₁₋₆ alkyl, or —N(R¹¹)₂;
each R¹⁰ is independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —OR¹¹, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl; and
each R¹¹ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆haloalkyl, C₁₋₆ alkoxy, C₁₋₆ haloalkoxy, C₃₋₁₀ cycloalkyl, aryl, heteroaryl, or heterocyclyl;
provided that:
when L is —NH—, R³ and R⁴ are both hydrogen, and the moiety

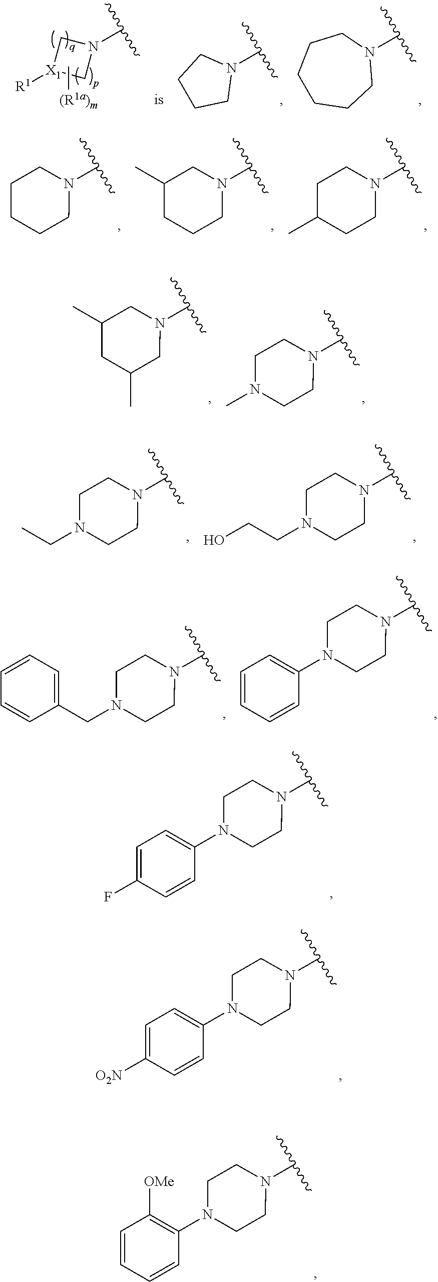

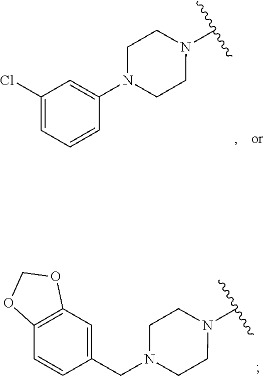 [SMILES: *N1CCN(c2cccc(Cl)c2)CC1 ; *N1CCN(Cc2ccc3c(c2)OCO3)CC1]

then the moiety

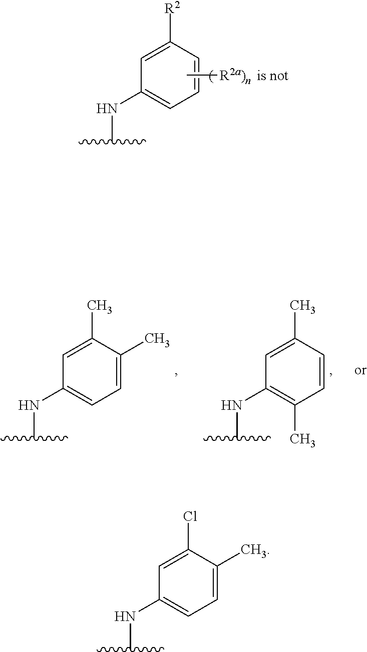

In another embodiment, provided is a pharmaceutical composition comprising a compound as described herein, or a pharmaceutically acceptable salt, isotopically enriched analog, stereoisomer, mixture of stereoisomers, or prodrug thereof, and a pharmaceutically acceptable excipient.

In another embodiment, provided is a method for treating a disease or disorder modulated, at least in part, by MutSβ, the method comprising administering to a subject in need thereof an effective amount of a compound disclosed herein, or a pharmaceutically acceptable salt, isotopically enriched analog, stereoisomer, mixture of stereoisomers, or prodrug thereof or a pharmaceutical composition as disclosed herein.

The disclosure also provides compositions, including pharmaceutical compositions, kits that include the compounds, or a pharmaceutically acceptable salt, isotopically enriched analog, stereoisomer, mixture of stereoisomers, or prodrug thereof, methods of using (or administering) and making the compounds, or a pharmaceutically acceptable salt, isotopically enriched analog, stereoisomer, mixture of stereoisomers, or prodrug thereof, and intermediates thereof.

The disclosure further provides compounds, or a pharmaceutically acceptable salt, isotopically enriched analog, stereoisomer, mixture of stereoisomers, or prodrug thereof, or compositions thereof for use in a method of treating a disease, disorder, or condition that is mediated, at least in part, by MutSβ.

Moreover, the disclosure provides uses of the compounds, or a pharmaceutically acceptable salt, isotopically enriched analog, stereoisomer, mixture of stereoisomers, or prodrug thereof, or compositions thereof in the manufacture of a medicament for the treatment of a disease, disorder, or condition that is mediated, at least in part, by MutSβ.

### DESCRIPTION

The description herein sets forth exemplary embodiments of the present technology. It should be recognized, however, that such description is not intended as a limitation on the scope of the present disclosure but is instead provided as a description of exemplary embodiments.

### 1. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl) or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, a divalent heteroaryl group, etc., may also be referred to as an “alkylene” group or an “alkylenyl” group (for example, methylenyl, ethylenyl, and propylenyl), an “arylene” group or an “arylenyl” group (for example, phenylenyl or napthylenyl, or quinolinyl for heteroarylene), respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkenyl” refers to an alkyl group containing at least one (e.g., 1-3 or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Alkylthio” refers to the group “alkyl-S—”. “Alkylsulfinyl” refers to the group “alkyl-S(O)—”. “Alkylsulfonyl” refers to the group “alkyl-S(O)₂—”.

“Acyl” refers to a group —C(O)R^{y}, wherein R^{y} is hydrogen, alkyl, alkenyl, alkynyl, cycloalkyl, heterocyclyl, aryl, heteroalkyl, or heteroaryl; each of which may be optionally substituted, as defined herein. Examples of acyl include, e.g., formyl, acetyl, cyclohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Arylalkyl” or “Aralkyl” refers to the group “aryl-alkyl-”.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl, or heteroaryl; each of which may be optionally substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₂ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Cycloalkylalkyl” refers to the group “cycloalkyl-alkyl-”.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Halogen” or “halo” refers to atoms occupying group VIIA of the periodic table, such as fluoro, chloro, bromo, or iodo.

“Haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6 or 1 to 3) hydrogen atoms are replaced by a halogen.

“Hydroxyalkyl” refers to an alkyl group as defined above, wherein one or more (e.g., 1 to 6 or 1 to 3) hydrogen atoms are replaced by a hydroxy group.

“Heteroalkyl” refers to an alkyl group in which one or more of the carbon atoms (and any associated hydrogen atoms), excluding any terminal carbon atom(s), are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl, or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.), and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkyl includes 2 to 10 carbon atoms, 2 to 8 carbon atoms, or 2 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, thiophenyl (i.e., thienyl), triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) or N-oxide (—O⁻) moieties Any non-aromatic ring or fused ring system containing at least one heteroatom and one non-aromatic ring is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. For example, fused ring systems such as decahydroquinazolinyl, 1,2,3,4-tetrahydroquinazolinyl, and 5,6,7,8-tetrahydroquinazolinyl are heterocyclyl, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Heterocyclylalkyl” refers to the group “heterocyclyl-alkyl-.”

“Oxime” refers to the group —CR^{y}(═NOH) wherein R^{y} is hydrogen, alkyl, alkenyl, alkynyl, cycloalkyl, heterocyclyl, aryl, heteroalkyl, or heteroaryl; each of which may be optionally substituted, as defined herein.

“Sulfonyl” refers to the group —S(O)₂R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfonyl are methylsulfonyl, ethylsulfonyl, phenylsulfonyl, and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfinyl are methylsulfinyl, ethylsulfinyl, phenylsulfinyl, and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5 or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5 or 1 to 3) hydrogen atom is replaced by a bond to a non-hydrogen atom such as, but not limited to alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanadino, halo, haloalkyl, haloalkoxy, hydroxyalkyl, heteroalkyl, heteroaryl, heteroarylalkyl, heterocyclyl, heterocyclylalkyl, —NHNH₂, ═NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide, or —Si(R^{y})₃, wherein each R^{y} is independently hydrogen, alkyl, alkenyl, alkynyl, heteroalkyl, cycloalkyl, aryl, heteroaryl, or heterocyclyl.

In certain embodiments, “substituted” includes any of the above alkyl, alkenyl, alkynyl, cycloalkyl, heterocyclyl, aryl, or heteroaryl groups in which one or more (e.g., 1 to 5 or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(O)R^{h}, —NR^{g}C(O)NR^{g}R^{h}, —NR^{g}C(O)OR^{h}, —NR^{g}S(O)₁₋₂R^{h}, —C(O)R^{g}, —C(O)OR^{g}, —OC(O)OR^{g}, —OC(O)R^{g}, —C(O)NR^{g}R^{h}, —OC(O)NR^{g}R^{h}, —OR^{g}, —SR^{g}, —S(O)R^{g}, —S(O)₂R^{g}, —OS(O)₁₋₂R^{g}, —S(O)₁₋₂OR^{g}, —NR^{g}S(O)₁₋₂NR^{g}R^{h}, ═NSO₂R^{g}, ═NOR^{g}, —S(O)₁₋₂NR^{g}R^{h}, —SF₅, —SCF₃, or —OCF₃. In certain embodiments, “substituted” also means any of the above groups in which one or more (e.g., 1 to 5 or 1 to 3) hydrogen atoms are replaced with —C(O)R^{g}, —C(O)OR⁹, —C(O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5 or 1 to 3) hydrogen atoms are replaced by a bond to an amino, cyano, hydroxy, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or R^{g} and R^{h} are taken together with the atoms to which they are attached to form a heterocyclyl ring optionally substituted with oxo, halo, or alkyl optionally substituted with oxo, halo, amino, hydroxy, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to three.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure include, for example, those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example, by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example, increased in vivo half-life, reduced dosage requirements, and/or an improvement in therapeutic index. An ¹⁸F, ³H, or ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino, and/or carboxyl groups, or groups similar thereto.

Provided is also a pharmaceutically acceptable salt, isotopically enriched analog, deuterated analog, stereoisomer, mixture of stereoisomers, or prodrugs of the compounds described herein. “Pharmaceutically acceptable” or “physiologically acceptable” refer to compounds, salts, compositions, dosage forms, and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃), mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds of the disclosure, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and/or fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers, or mixtures thereof, and includes “enantiomers,” which refers to two stereoisomers whose molecules are nonsuperimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds described herein, and the like. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

### 2. Compounds

Provided herein are compounds that are inhibitors of MutSβ. In certain embodiments, provided is a compound of Formula I:

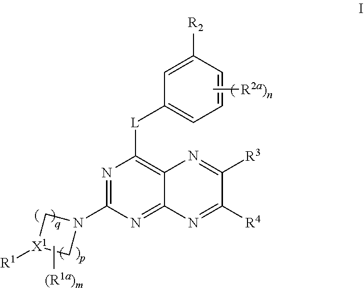

or a pharmaceutically acceptable salt, isotopically enriched analog, stereoisomer, mixture of stereoisomers, or prodrug thereof, wherein:
n is 0, 1, 2, 3, or 4;
m is 0, 1, 2, 3, 4, or 5;
p is 0, 1, 2, 3, 4, or 5;
q is 0, 1, 2, 3, or 4; provided that p+q is 2, 3, 4, or 5;
L is —NH—, —O—, —CH₂—, —NHCH₂—, —OCH₂—, or —CH₂CH₂—;
X¹ is CR⁵ or N;
R¹ is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 R¹⁰;
R^{1a} and R^{2a} are each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 R¹⁰;
R² is halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 R¹⁰;
R³ and R⁴ are each independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 R¹⁰;
R⁵ is hydrogen, C₁₋₆ alkyl, or —N(R¹¹)₂;
each R¹⁰ is independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —OR¹¹, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl; and
each R¹¹ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl;
provided that:
when L is —NH—, R³ and R⁴ are both hydrogen, and the moiety

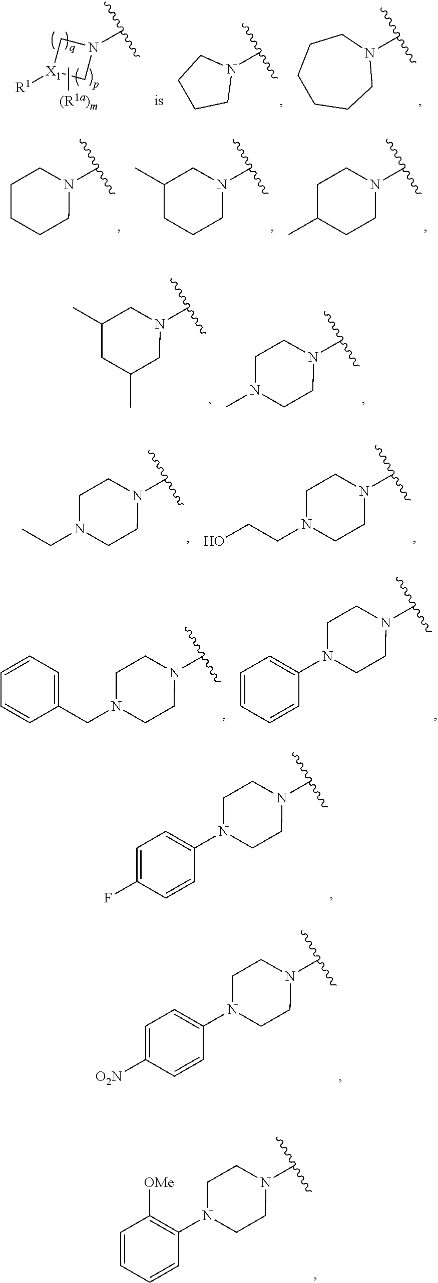

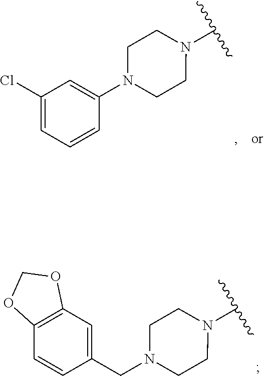 [SMILES: *N1CCN(c2cccc(Cl)c2)CC1 ; *N1CCN(Cc2ccc3c(c2)OCO3)CC1]

then the moiety

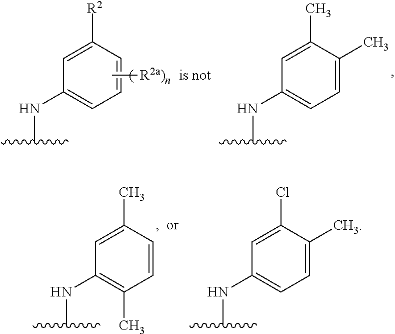

In certain embodiments, p is 0. In certain embodiments, p is 1. In certain embodiments, p is 2. In certain embodiments, p is 3. In certain embodiments, p is 4. In certain embodiments, p is 5.

In certain embodiments, q is 0. In certain embodiments, q is 1. In certain embodiments, q is 2. In certain embodiments, q is 3. In certain embodiments, q is 4.

In certain embodiments, p+q is 2. In certain embodiments, p+q is 3 or 4. In certain embodiments, p+q is 3. In certain embodiments, p+q is 4. In certain embodiments, p+q is 5.

In certain embodiments, p is 0 and q is 2. In certain embodiments, p is 0 and q is 3. In certain embodiments, p is 0 and q is 4. In certain embodiments, p is 1 and q is 1. In certain embodiments, p is 1 and q is 2. In certain embodiments, p is 1 and q is 3. In certain embodiments, p is 1 and q is 4. In certain embodiments, p is 2 and q is 0. In certain embodiments, p is 2 and q is 1. In certain embodiments, p is 2 and q is 2. In certain embodiments, p is 2 and q is 3. In certain embodiments, p is 3 and q is 0. In certain embodiments, p is 3 and q is 1. In certain embodiments, p is 3 and q is 2. In certain embodiments, p is 4 and q is 0. In certain embodiments, p is 4 and q is 1. In certain embodiments, p is 5 and q is 0.

In certain embodiments, the moiety

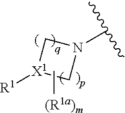

is

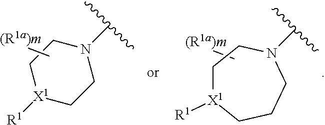

In certain embodiments, the moiety

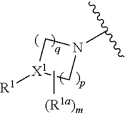

is

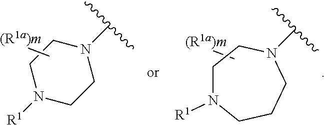

In certain embodiments, the moiety

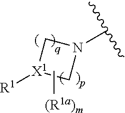

is

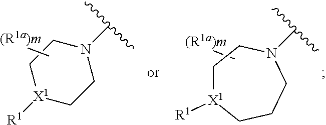

and R³ and R⁴ are each independently hydrogen or C₁₋₃ alkyl.

In certain embodiments, the moiety

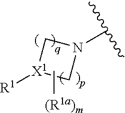

is

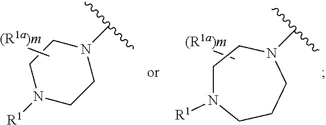

and R³ and R⁴ are each independently hydrogen or C₁₋₃ alkyl.

In certain embodiments, the moiety

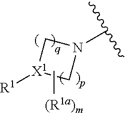

is

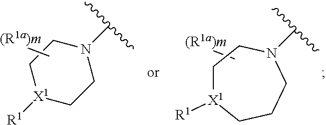

R¹ is C₁₋₃ alkyl; and each R^{1a} is independently C₁₋₃ alkyl.

In certain embodiments, the moiety

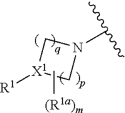

is

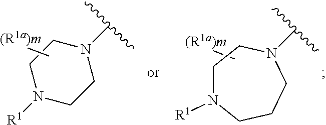

R¹ is C₁₋₃ alkyl; and each R^{1a} is independently C₁₋₃ alkyl.

In certain embodiments, the moiety

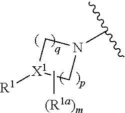

is

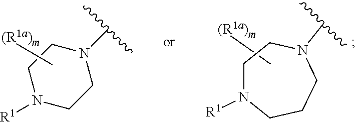

R¹ is C₁₋₃ alkyl; and
R³ and R⁴ are each independently hydrogen or C₁₋₃ alkyl.

In certain embodiments, the moiety

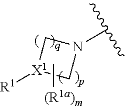

is

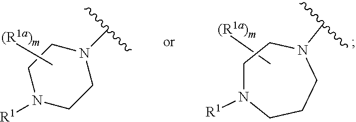

n is 0 or 1;
m is 0 or 1;
R¹ is C₁₋₃ alkyl; and
R³ and R⁴ are each independently hydrogen or C₁₋₃ alkyl.

In certain embodiments, the moiety

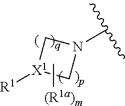

is

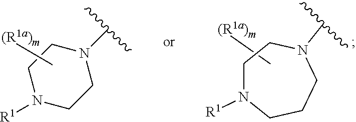

n is 0 or 1;
m is 0 or 1;

### L is —NH—, —O—, or —NHCH₂—;

R¹ is C₁₋₃ alkyl; and
R³ and R⁴ are each independently hydrogen or C₁₋₃ alkyl.

In certain embodiments, the moiety

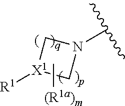

is

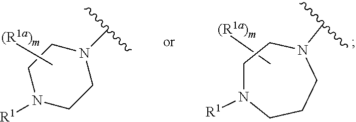

n is 0 or 1;
m is 0 or 1;

### L is —NH—, —O—, or —NHCH₂—;

R¹ is C₁₋₃ alkyl;
R^{1a} is C₁₋₃ alkyl; and
R³ and R⁴ are each independently hydrogen or C₁₋₃ alkyl.

In certain embodiments provided is a compound of Formula IA:

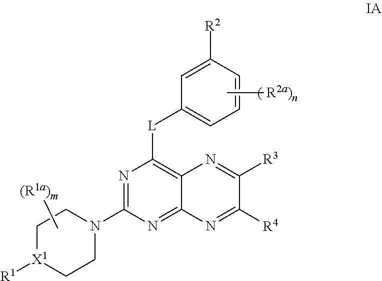

or a pharmaceutically acceptable salt, isotopically enriched analog, stereoisomer, mixture of stereoisomers, or prodrug thereof.

In certain embodiments, X¹ is N.

In certain embodiments, X¹ is CR⁵.

In certain embodiments, R⁵ is hydrogen. In certain embodiments, R⁵ is C₁₋₆ alkyl. In certain embodiments, R⁵ is —N(R¹¹)₂.

In certain embodiments, R¹ is hydrogen, halo, or cyano. In certain embodiments, R¹ is hydrogen. In certain embodiments, R¹ is halo. In certain embodiments, R¹ is cyano.

In certain embodiments, R¹ is C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰.

In certain embodiments, R¹ is C₂₋₆ alkenyl optionally substituted with 1-5 R¹⁰.

In certain embodiments, R¹ is C₂₋₆ alkynyl optionally substituted with 1-5 R¹⁰.

In certain embodiments, R¹ is C₁₋₆ haloalkyl optionally substituted with 1-5 R¹⁰.

In certain embodiments, R¹ is C₃₋₁₀ cycloalkyl optionally substituted with 1-5 R¹⁰.

In certain embodiments, R¹ is C₃₋₁₀ cycloalkenyl optionally substituted with 1-5 R¹⁰.

In certain embodiments, R¹ is heterocyclyl optionally substituted with 1-5 R¹⁰.

In certain embodiments, R¹ is aryl optionally substituted with 1-5 R¹⁰.

In certain embodiments, R¹ is heteroaryl optionally substituted with 1-5 R¹⁰.

In certain embodiments, R¹ is C₁₋₆ alkyl optionally substituted with 1-5 R¹⁰.

In certain embodiments, R¹ is C₁₋₆ alkyl. In certain embodiments, R¹ is C₁₋₃ alkyl. In certain embodiments, R¹ is methyl. In certain embodiments, R¹ is ethyl. In certain embodiments, R¹ is isopropyl.

In certain embodiments, X¹ is N and R¹ is C₁₋₆ alkyl. In certain embodiments, X¹ is N and R¹ is methyl. In certain embodiments, X¹ is N and R¹ is ethyl. In certain embodiments, X¹ is N and R¹ is isopropyl.

In certain embodiments, provided is a compound of Formula IB:

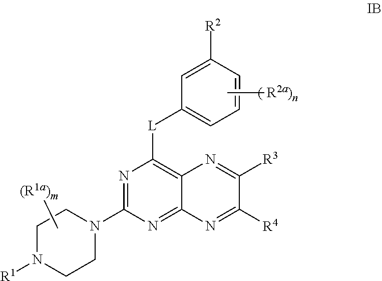

or a pharmaceutically acceptable salt, isotopically enriched analog, stereoisomer, mixture of stereoisomers, or prodrug thereof.

In certain embodiments, L is —NH—, —O—, or —NHCH₂—. In certain embodiments, L is —NH—. In certain embodiments, L is —O—. In certain embodiments, L is —CH₂—. In certain embodiments, L is —NHCH₂—. In certain embodiments, L is —OCH₂—. In certain embodiments, L is —CH₂CH₂—.

In certain embodiments, provided is a compound of Formula IC:

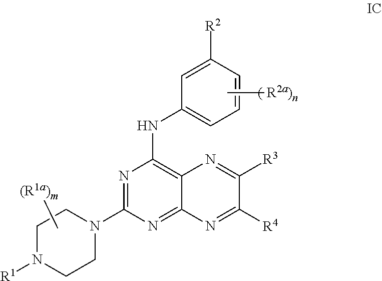

or a pharmaceutically acceptable salt, isotopically enriched analog, stereoisomer, mixture of stereoisomers, or prodrug thereof.

In certain embodiments, n is 0. In certain embodiments, n is 1. In certain embodiments, n is 2. In certain embodiments, n is 3. In certain embodiments, n is 4. In certain embodiments, n is 0 or 1.

In certain embodiments, provided is a compound of Formula ID:

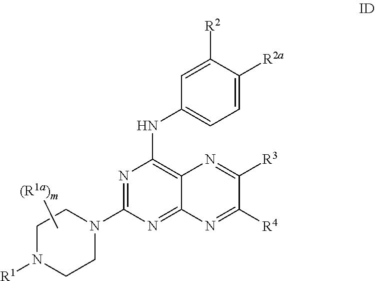

or a pharmaceutically acceptable salt, isotopically enriched analog, stereoisomer, mixture of stereoisomers, or prodrug thereof.

In certain embodiments, provided is a compound of formula IE:

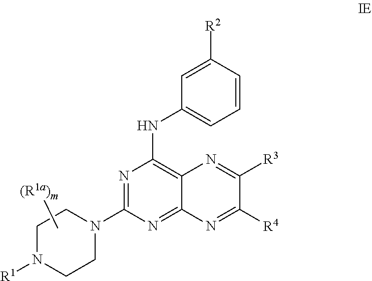

or a pharmaceutically acceptable salt, isotopically enriched analog, stereoisomer, mixture of stereoisomers, or prodrug thereof.

In certain embodiments, provided is a compound of formula IF:

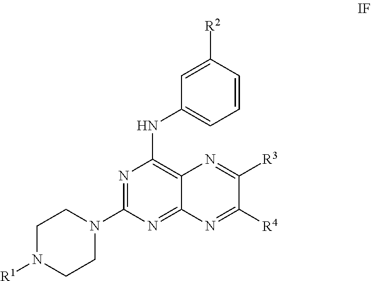

or a pharmaceutically acceptable salt, isotopically enriched analog, stereoisomer, mixture of stereoisomers, or prodrug thereof.

In certain embodiments, R² and R^{2a} are each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰.

In certain embodiments, R² and R^{2a} are each independently halo, cyano, C₁₋₆ alkyl, C₁₋₆ haloalkyl, C₃₋₁₀ cycloalkyl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₁₋₆ haloalkyl, or C₃₋₁₀ cycloalkyl is independently optionally substituted with 1-5 R¹⁰.

In certain embodiments, R² and R^{2a} are each independently halo, cyano, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹.

In certain embodiments, R² and R^{2a} are each independently halo.

In certain embodiments, R² and R^{2a} are each independently fluoro or chloro. In certain embodiments, R² and R^{2a} are fluoro. In certain embodiments, R² and R^{2a} are chloro.

In certain embodiments, R² and R^{2a} are each independently C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰. In certain embodiments, R² and R^{2a} are each independently C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 R¹⁰.

In certain embodiments, R² and R^{2a} are each independently C₁₋₆ alkyl.

In certain embodiments, R² and R^{2a} are methyl.

In certain embodiments, R² and R^{2a} are each independently halo or C₁₋₆ alkyl. In certain embodiments, R² and R^{2a} are each independently halo or methyl.

In certain embodiments, R² and R^{2a} are each independently fluoro, chloro, or methyl. In certain embodiments, R² is fluoro and R^{2a} is methyl. In certain embodiments, R² is methyl and R^{2a} is fluoro. In certain embodiments, R² is chloro and R^{2a} is methyl. In certain embodiments, R² is methyl and R^{2a} is chloro.

In certain embodiments, the moiety

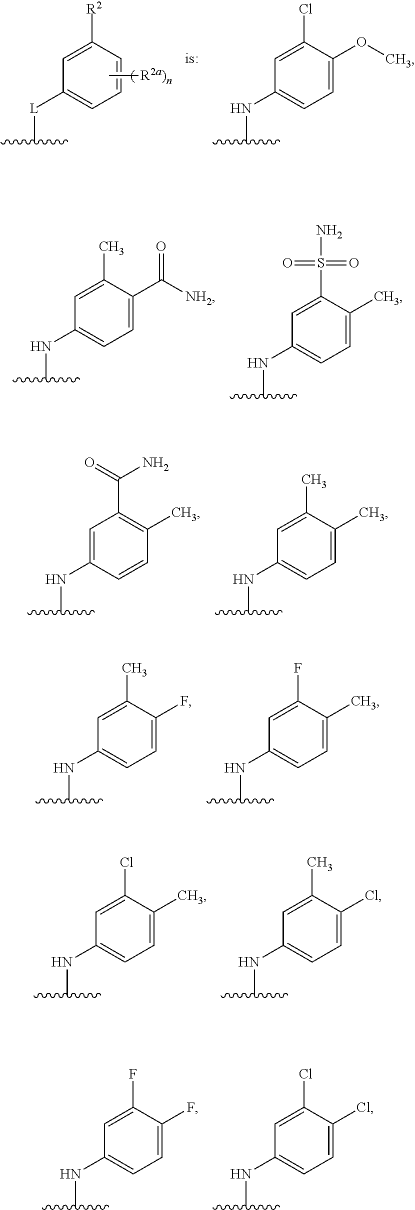

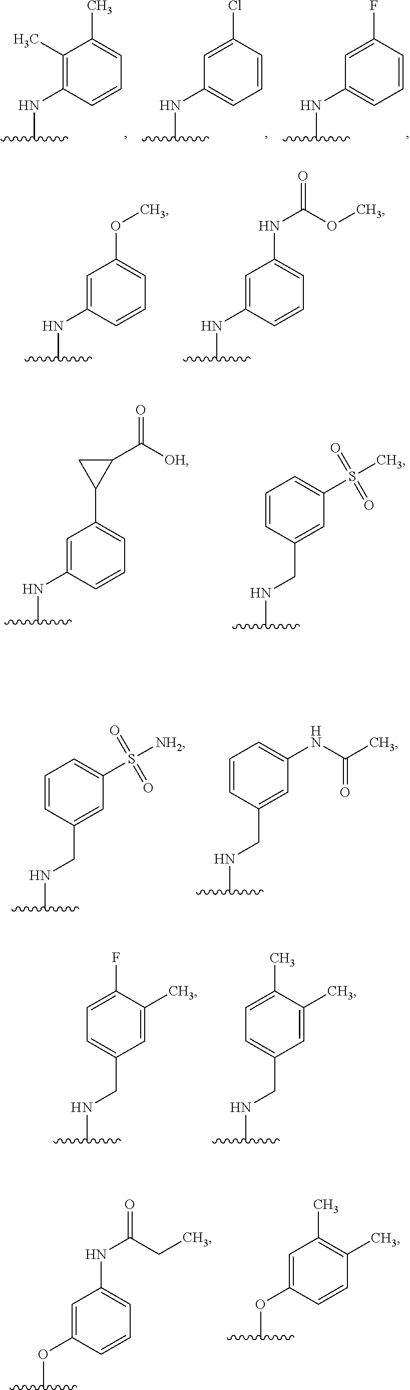

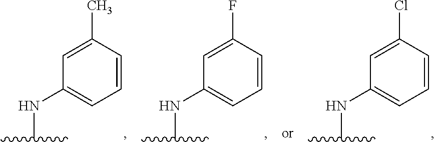

In certain embodiments, the moiety

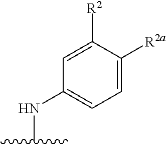

is

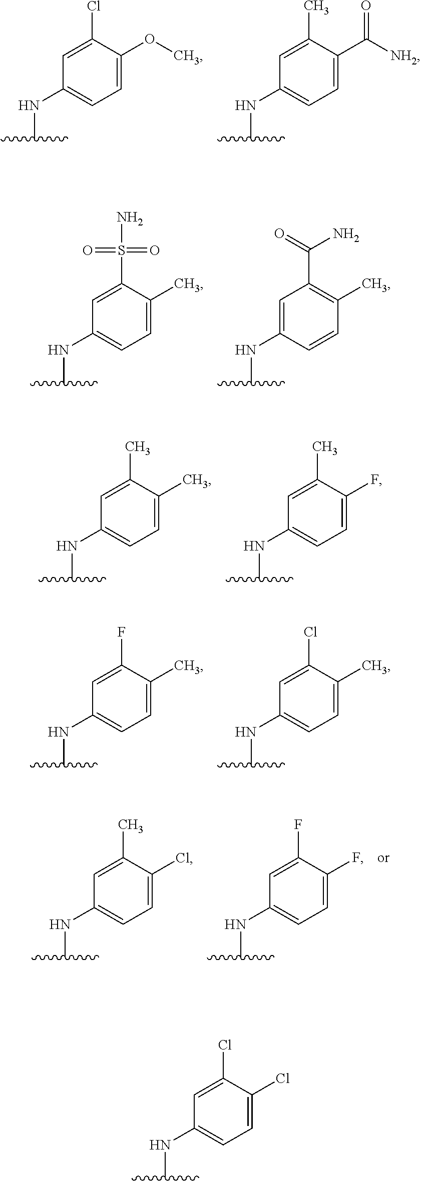

In certain embodiments, the moiety

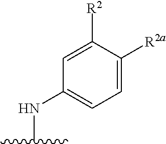

is:

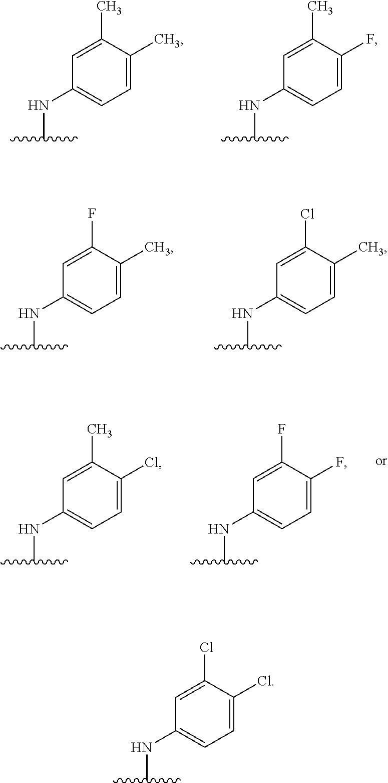

In certain embodiments, n is 0 and R² is methyl. In certain embodiments, n is 0 and R² is fluoro. In certain embodiments, n is 0 and R² is chloro.

In certain embodiments, the moiety

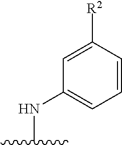

is:

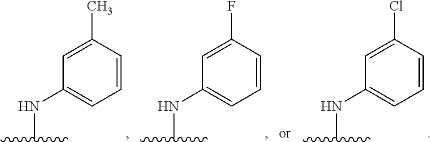

In certain embodiments, R³ and R⁴ are each independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰.

In certain embodiments, R³ and R⁴ are each independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 R¹⁰.

In certain embodiments, R³ and R⁴ are each independently hydrogen, halo, cyano, or C₁₋₆ alkyl, In certain embodiments, R³ and R⁴ are each independently hydrogen, halo, cyano, or C₁₋₃ alkyl.

In certain embodiments, R³ and R⁴ are each independently hydrogen, halo, or cyano.

In certain embodiments, R³ and R⁴ are each independently hydrogen or C₁₋₃ alkyl.

In certain embodiments, R³ and R⁴ are each independently hydrogen.

In certain embodiments, R³ is hydrogen.

In certain embodiments, R⁴ is hydrogen.

In certain embodiments, R^{1a} is each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰.

In certain embodiments, R^{1a} is each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl, wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰.

In certain embodiments, R^{1a} is halo. In certain embodiments, R^{1a} is cyano. In certain embodiments, R^{1a} is C₁₋₆ alkyl optionally substituted with 1-5 R¹⁰. In certain embodiments, R^{1a} is C₁₋₃ alkyl optionally substituted with 1-5 R¹⁰. In certain embodiments, R^{1a} is C₂₋₆ alkenyl optionally substituted with 1-5 R¹⁰. In certain embodiments, R^{1a} is C₂₋₆ alkynyl optionally substituted with 1-5 R¹⁰. In certain embodiments, R^{1a} is C₁₋₆ haloalkyl optionally substituted with 1-5 R¹⁰. In certain embodiments, R^{1a} is C₃₋₁₀ cycloalkyl optionally substituted with 1-5 R¹⁰. In certain embodiments, R^{1a} is C₃₋₁₀ cycloalkenyl optionally substituted with 1-5 R¹⁰. In certain embodiments, R^{1a} is heterocyclyl optionally substituted with 1-5 R¹⁰. In certain embodiments, R^{1a} is aryl optionally substituted with 1-5 R¹⁰. In certain embodiments, R^{1a} is heteroaryl optionally substituted with 1-5 R¹⁰.

In certain embodiments, m is 0. In certain embodiments, m is 1. In certain embodiments, m is 2. In certain embodiments, m is 3. In certain embodiments, m is 4. In certain embodiments, m is 5. In certain embodiments, m is 0 or 1.

In certain embodiments, provided is a compound of Formula IG:

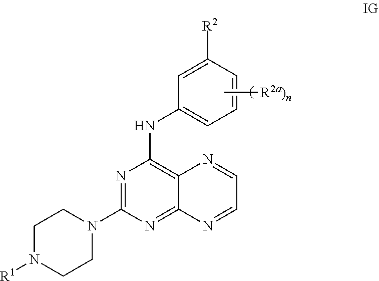

or a pharmaceutically acceptable salt, isotopically enriched analog, stereoisomer, mixture of stereoisomers, or prodrug thereof, wherein R¹, R², R^{2a}, and n are as described herein.

In certain embodiments, each R¹⁰ is independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl, —OR¹¹, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹.

In certain embodiments, each R¹⁰ is independently C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl. In certain embodiments, each R¹⁰ is independently C₁₋₆ alkyl.

In certain embodiments, each R¹¹ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl. In certain embodiments, each R¹¹ is independently hydrogen or C₁₋₆ alkyl. In certain embodiments, each R¹¹ is independently hydrogen.

In certain embodiments, a compound or a pharmaceutically acceptable salt, isotopically enriched analog, stereoisomer, mixture of stereoisomers, or prodrug thereof, is selected from:

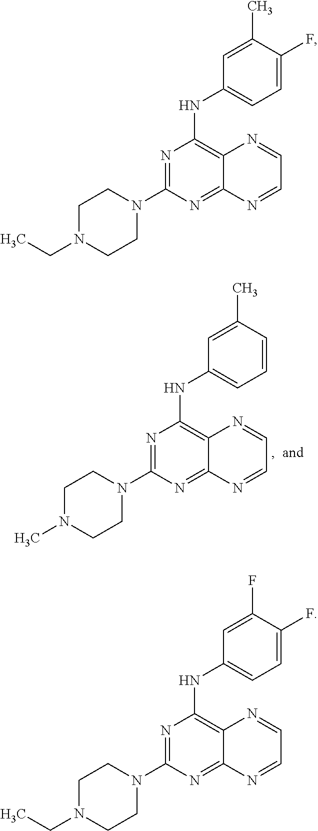 [SMILES: CCN1CCN(c2nc(Nc3ccc(F)c(C)c3)c3nccnc3n2)CC1 ; Cc1cccc(Nc2nc(N3CCN(C)CC3)nc3nccnc23)c1 ; CCN1CCN(c2nc(Nc3ccc(F)c(F)c3)c3nccnc3n2)CC1]

In certain embodiments, a compound or a pharmaceutically acceptable salt, isotopically enriched analog, stereoisomer, mixture of stereoisomers, or prodrug thereof, is selected from:

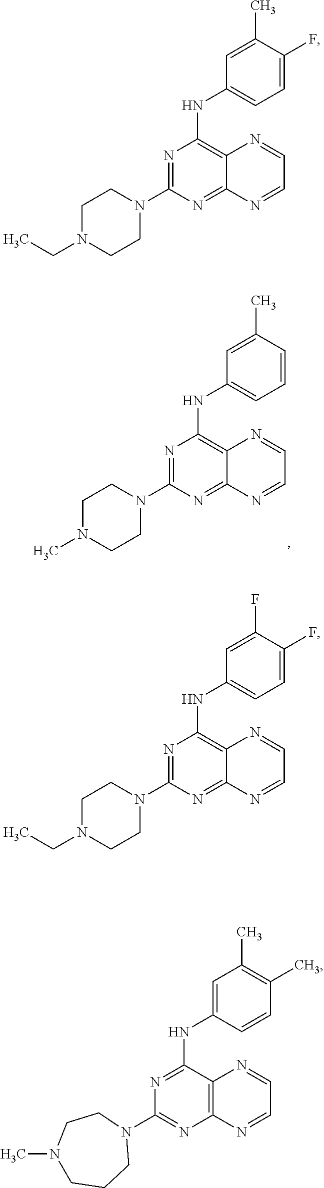 [SMILES: CCN1CCN(c2nc(Nc3ccc(F)c(C)c3)c3nccnc3n2)CC1 ; Cc1cccc(Nc2nc(N3CCN(C)CC3)nc3nccnc23)c1 ; CCN1CCN(c2nc(Nc3ccc(F)c(F)c3)c3nccnc3n2)CC1 ; Cc1ccc(Nc2nc(N3CCCN(C)CC3)nc3nccnc23)cc1C]

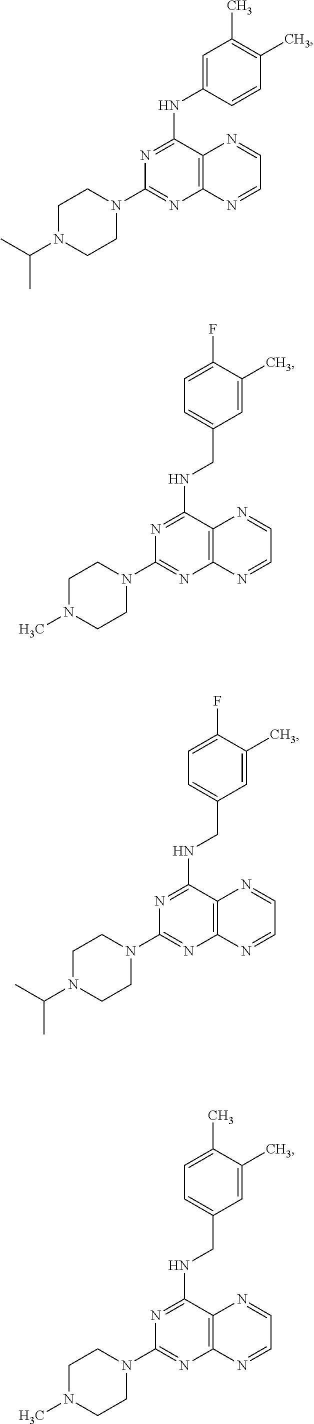 [SMILES: Cc1ccc(Nc2nc(N3CCN(C(C)C)CC3)nc3nccnc23)cc1C ; Cc1cc(CNc2nc(N3CCN(C)CC3)nc3nccnc23)ccc1F ; Cc1cc(CNc2nc(N3CCN(C(C)C)CC3)nc3nccnc23)ccc1F ; Cc1ccc(CNc2nc(N3CCN(C)CC3)nc3nccnc23)cc1C]

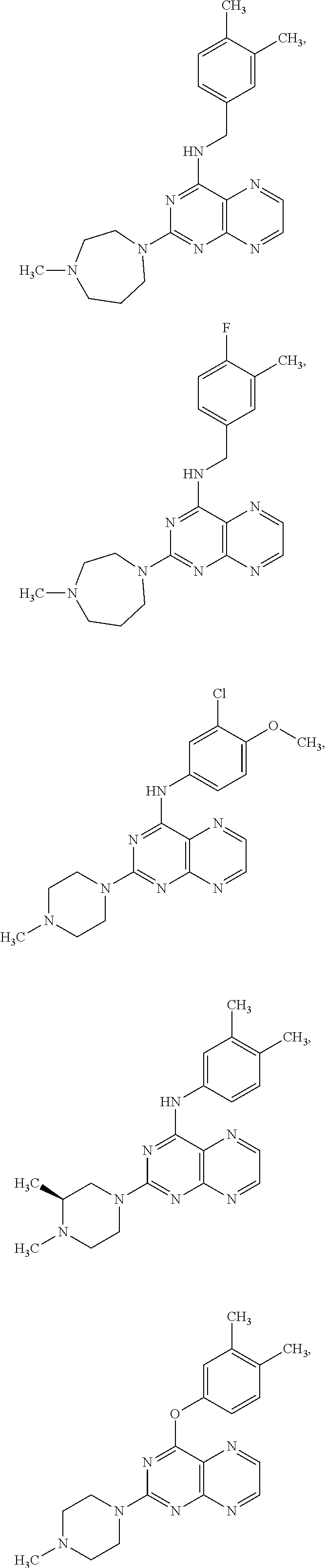 [SMILES: Cc1ccc(CNc2nc(N3CCCN(C)CC3)nc3nccnc23)cc1C ; Cc1cc(CNc2nc(N3CCCN(C)CC3)nc3nccnc23)ccc1F ; COc1ccc(Nc2nc(N3CCN(C)CC3)nc3nccnc23)cc1Cl ; Cc1ccc(Nc2nc(N3CCN(C)[C@@H](C)C3)nc3nccnc23)cc1C ; Cc1ccc(Oc2nc(N3CCN(C)CC3)nc3nccnc23)cc1C]

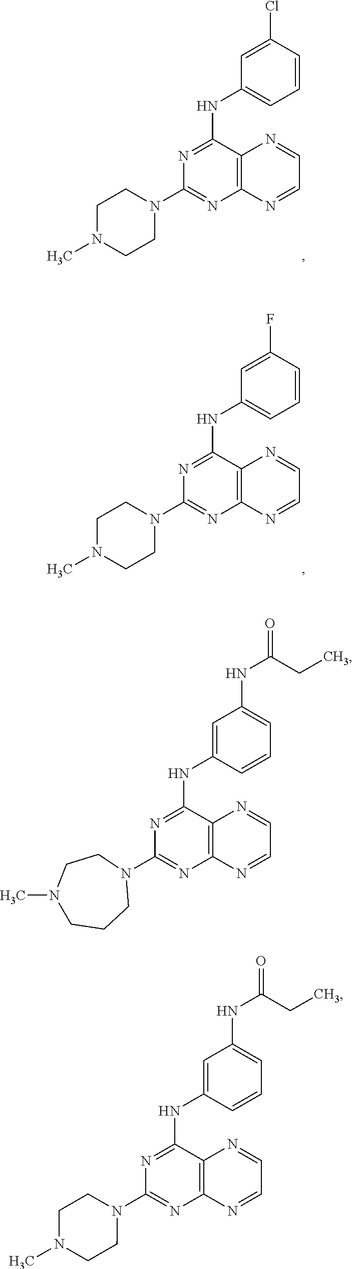 [SMILES: CN1CCN(c2nc(Nc3cccc(Cl)c3)c3nccnc3n2)CC1 ; CN1CCN(c2nc(Nc3cccc(F)c3)c3nccnc3n2)CC1 ; CCC(=O)Nc1cccc(Nc2nc(N3CCCN(C)CC3)nc3nccnc23)c1 ; CCC(=O)Nc1cccc(Nc2nc(N3CCN(C)CC3)nc3nccnc23)c1]

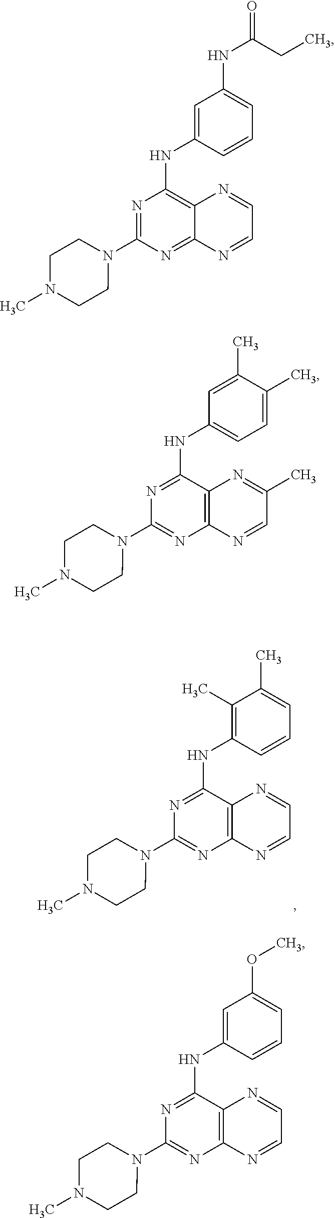

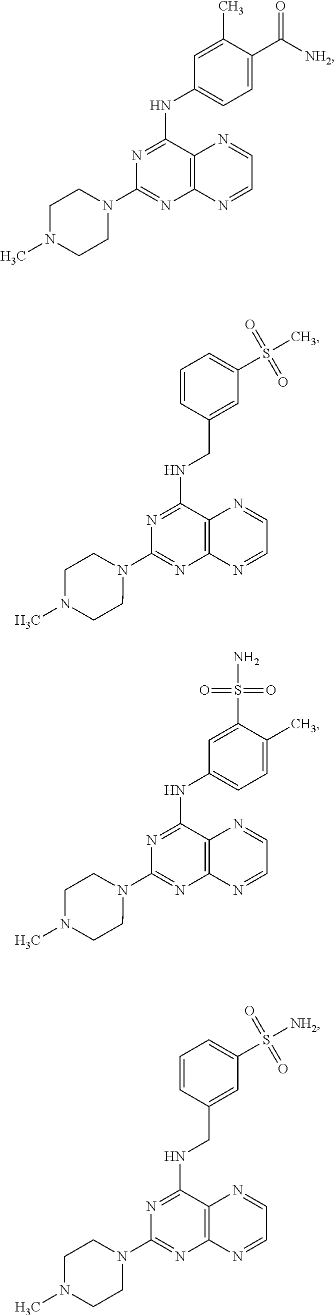 [SMILES: Cc1cc(Nc2nc(N3CCN(C)CC3)nc3nccnc23)ccc1C(N)=O ; CN1CCN(c2nc(NCc3cccc(S(C)(=O)=O)c3)c3nccnc3n2)CC1 ; Cc1ccc(Nc2nc(N3CCN(C)CC3)nc3nccnc23)cc1S(N)(=O)=O ; CN1CCN(c2nc(NCc3cccc(S(N)(=O)=O)c3)c3nccnc3n2)CC1]

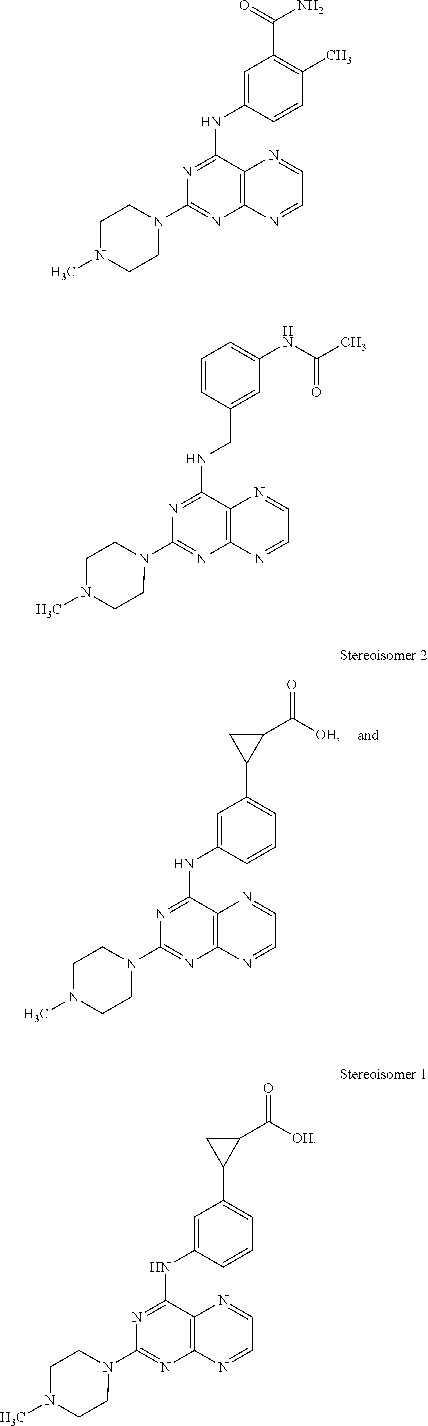

### 3. Methods

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in certain embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy, and/or veterinary applications. In certain embodiments, the subject is a mammal. In certain embodiments, the subject is a human.

The term “therapeutically effective amount” or “effective amount” of a compound described herein or a pharmaceutically acceptable salt, isotopically enriched analog, stereoisomer, mixture of stereoisomers, or prodrug thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition as described herein. The therapeutically effective amount may vary depending on the subject, and disease or condition being treated, the weight and age of the subject, the severity of the disease or condition, and the manner of administering, which can readily be determined by one of ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The compounds may be further characterized to examine the safety or tolerance dosage in human or non-human subjects. Such properties may be examined using commonly known methods to those skilled in the art.

In certain embodiments, provided are compounds, or a pharmaceutically acceptable salt, isotopically enriched analog, stereoisomer, mixture of stereoisomers, or prodrug thereof, that inhibit the activity of the MutSβ complex. In certain embodiments, the compounds provided herein, or a pharmaceutically acceptable salt, isotopically enriched analog, stereoisomer, mixture of stereoisomers, or prodrug thereof, inhibits MutSβ.

In certain methods, uses and compositions provided herein, the compound is a compound of Formula I:

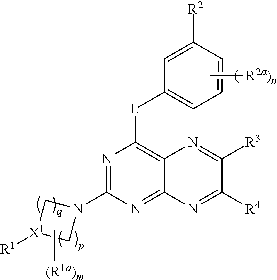

or a pharmaceutically acceptable salt, isotopically enriched analog, stereoisomer, mixture of stereoisomers, or prodrug thereof, wherein:
n is 0, 1, 2, 3, or 4;
m is 0, 1, 2, 3, 4, or 5;
p is 0, 1, 2, 3, 4, or 5;
q is 0, 1, 2, 3, or 4; provided that p+q is 2, 3, 4, or 5;
L is —NH—, —O—, —CH₂—, —NHCH₂—, —OCH₂—, or —CH₂CH₂—;
X¹ is O, N, or CH; provided that when X¹ is O, then R¹ is absent;
R¹, when present, is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R^{1a} and R^{2a} are each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R² is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R³ and R⁴ are each independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
each R¹⁰ is independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —OR¹¹, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl; and
each R¹¹ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl.

In certain methods, uses and compositions provided herein, the compound is a compound of Formula I:

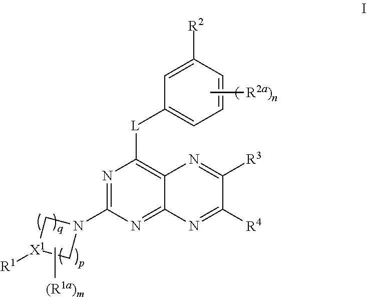

or a pharmaceutically acceptable salt, isotopically enriched analog, stereoisomer, mixture of stereoisomers, or prodrug thereof, wherein:
n is 0, 1, 2, 3, or 4;
m is 0, 1, 2, 3, 4, or 5;
p is 0, 1, 2, 3, 4, or 5;
q is 0, 1, 2, 3, or 4; provided that p+q is 2, 3, 4, or 5;
L is —NH—, —O—, —CH₂—, —NHCH₂—, —OCH₂—, or —CH₂CH₂—;
X¹ is O, N, or CH; provided that when X¹ is O, then R¹ is absent;
R¹, when present, is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 R¹⁰;
R^{1a} and R^{2a} are each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 R¹⁰;
R² is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 R¹⁰;
R³ and R⁴ are each independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 R¹⁰;
each R¹⁰ is independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —OR¹¹, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl; and
each R¹¹ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl.

In certain embodiments, the compound, or pharmaceutically acceptable salt, isotopically enriched analog, stereoisomer, mixture of stereoisomers, or prodrug thereof, is selected from Table 1.

TABLE 1

| Compound | |
|---|---|
| No. | Structure |
| 1 | 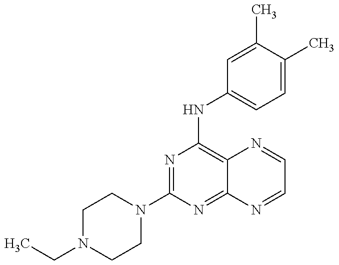 [SMILES: CCN1CCN(c2nc(Nc3ccc(C)c(C)c3)c3nccnc3n2)CC1] |
| 2 | 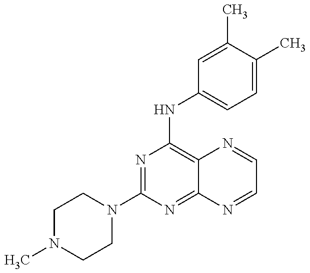 |
| 3 | 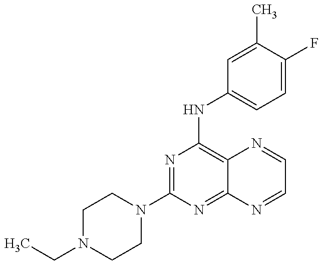 [SMILES: CCN1CCN(c2nc(Nc3ccc(F)c(C)c3)c3nccnc3n2)CC1] |
| 4 | 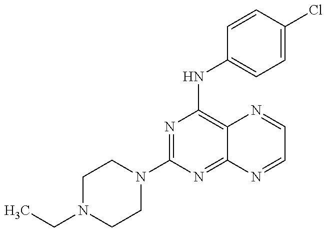 [SMILES: CCN1CCN(c2nc(Nc3ccc(Cl)cc3)c3nccnc3n2)CC1] |
| 5 | 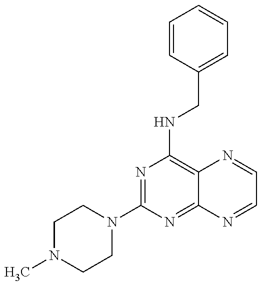 [SMILES: CN1CCN(c2nc(NCc3ccccc3)c3nccnc3n2)CC1] |
| 6 | 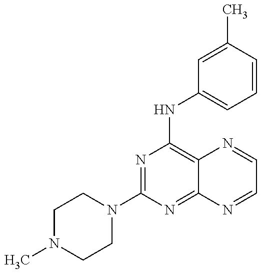 [SMILES: Cc1cccc(Nc2nc(N3CCN(C)CC3)nc3nccnc23)c1] |
| 7 | 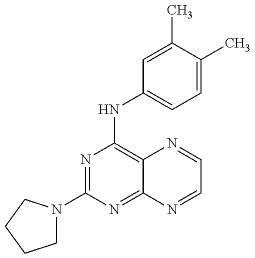 [SMILES: Cc1ccc(Nc2nc(N3CCCC3)nc3nccnc23)cc1C] |
| 8 | 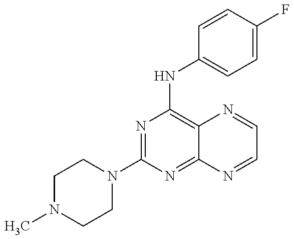 [SMILES: CN1CCN(c2nc(Nc3ccc(F)cc3)c3nccnc3n2)CC1] |
| 9 | 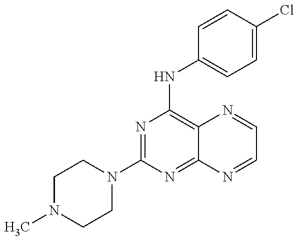 [SMILES: CN1CCN(c2nc(Nc3ccc(Cl)cc3)c3nccnc3n2)CC1] |
| 10 | 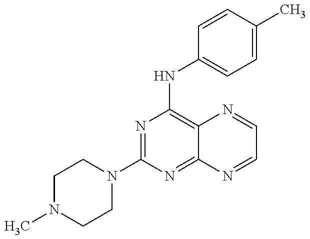 |
| 16 | 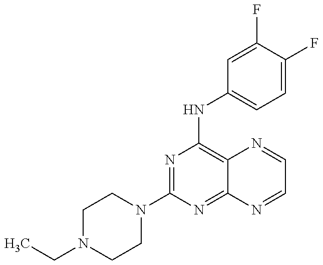 [SMILES: CCN1CCN(c2nc(Nc3ccc(F)c(F)c3)c3nccnc3n2)CC1] |
| 18 | 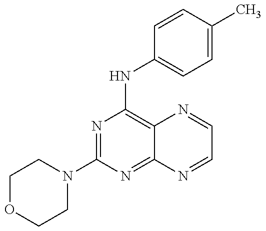 [SMILES: Cc1ccc(Nc2nc(N3CCOCC3)nc3nccnc23)cc1] |
| 20 | 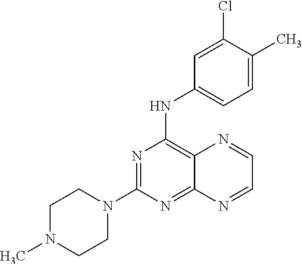 |
| 22 | 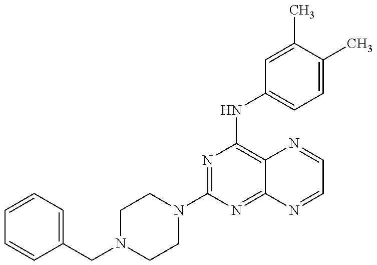 [SMILES: Cc1ccc(Nc2nc(N3CCN(Cc4ccccc4)CC3)nc3nccnc23)cc1C] |
| 23 | 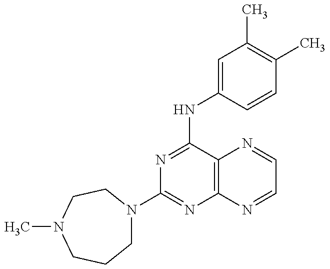 |
| 24 | 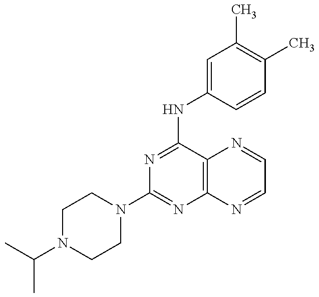 [SMILES: Cc1ccc(Nc2nc(N3CCN(C(C)C)CC3)nc3nccnc23)cc1C] |
| 25 | 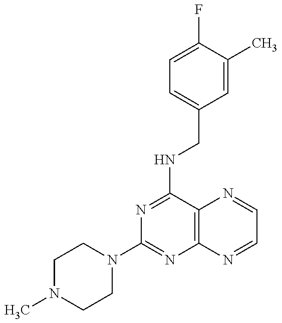 |
| 26 | 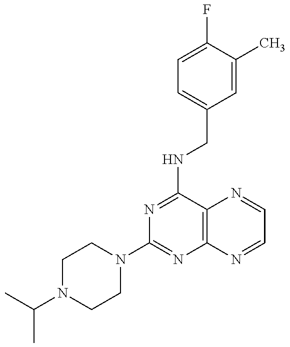 [SMILES: Cc1cc(CNc2nc(N3CCN(C(C)C)CC3)nc3nccnc23)ccc1F] |
| 27 | 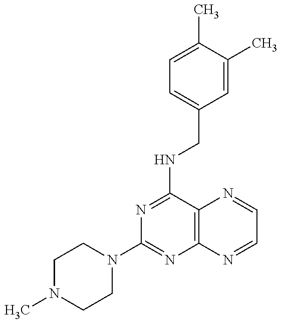 |
| 28 | 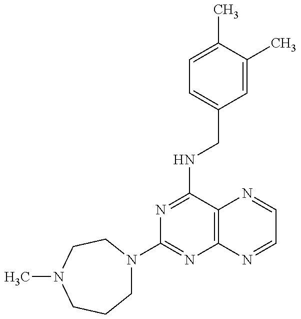 |
| 29 | 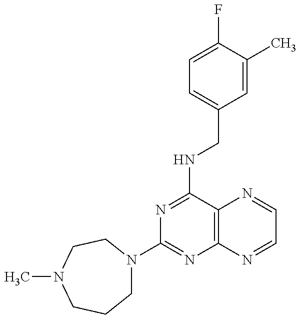 |
| 30 | 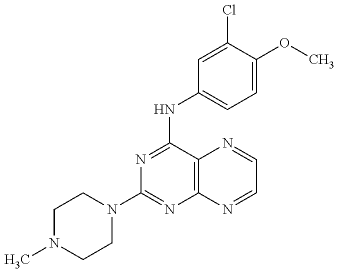 |
| 31 | 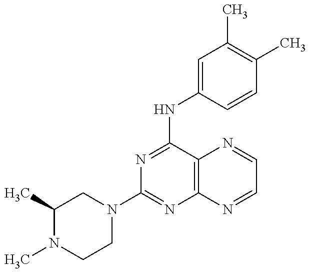 |
| 32 | 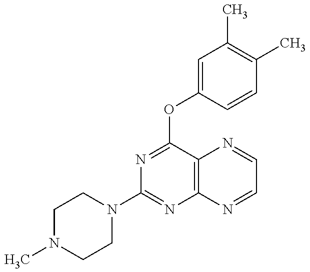 |
| 33 | 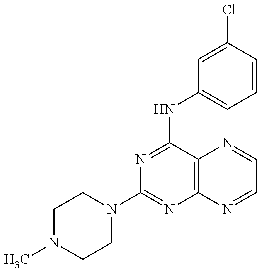 [SMILES: CN1CCN(c2nc(Nc3cccc(Cl)c3)c3nccnc3n2)CC1] |
| 34 | 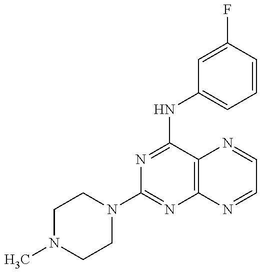 [SMILES: CN1CCN(c2nc(Nc3cccc(F)c3)c3nccnc3n2)CC1] |
| 35 | 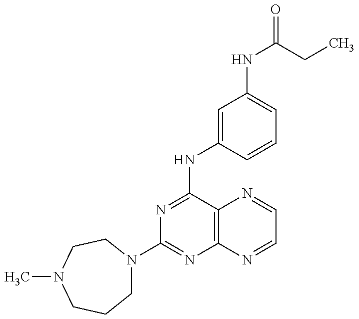 [SMILES: CCC(=O)Nc1cccc(Nc2nc(N3CCCN(C)CC3)nc3nccnc23)c1] |
| 36 | 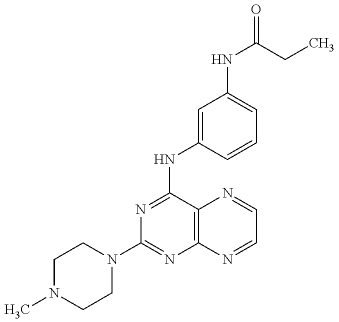 [SMILES: CCC(=O)Nc1cccc(Nc2nc(N3CCN(C)CC3)nc3nccnc23)c1] |
| 37 | 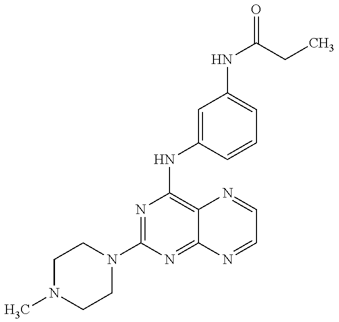 [SMILES: CCC(=O)Nc1cccc(Nc2nc(N3CCN(C)CC3)nc3nccnc23)c1] |
| 38 | 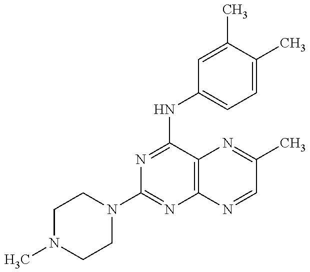 |
| 39 | 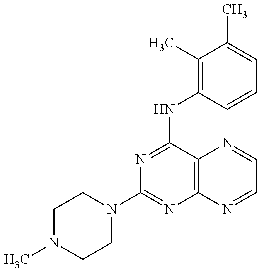 [SMILES: Cc1cccc(Nc2nc(N3CCN(C)CC3)nc3nccnc23)c1C] |
| 40 | 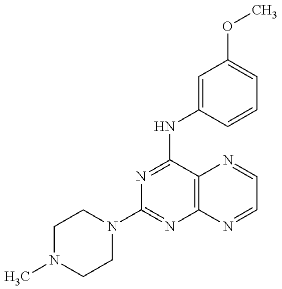 |
| 41 | 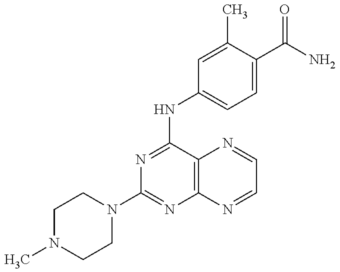 [SMILES: Cc1cc(Nc2nc(N3CCN(C)CC3)nc3nccnc23)ccc1C(N)=O] |
| 42 | 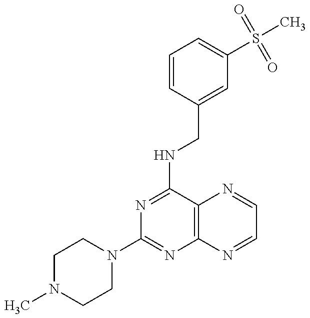 |
| 43 | 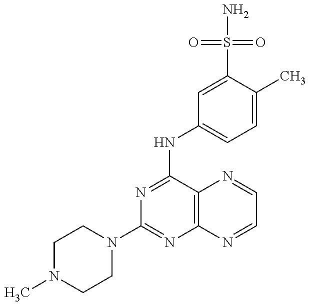 |
| 44 | 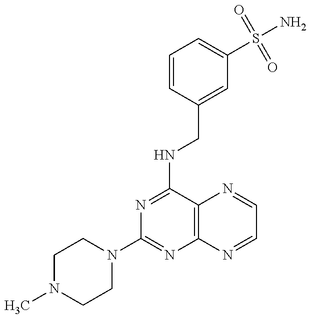 [SMILES: CN1CCN(c2nc(NCc3cccc(S(N)(=O)=O)c3)c3nccnc3n2)CC1] |
| 45 | 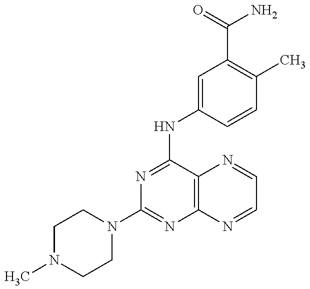 |
| 46 | 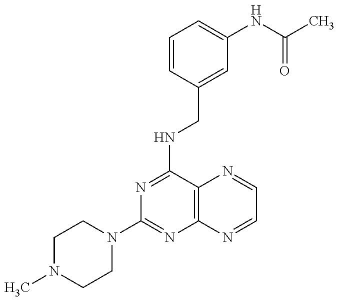 [SMILES: CC(=O)Nc1cccc(CNc2nc(N3CCN(C)CC3)nc3nccnc23)c1] |
| 47 | 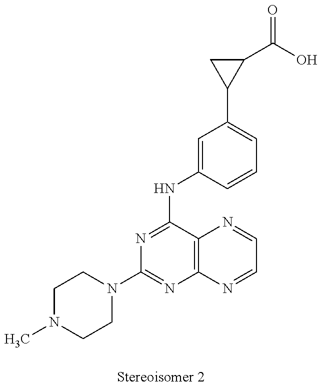 [SMILES: CN1CCN(c2nc(Nc3cccc(C4CC4C(=O)O)c3)c3nccnc3n2)CC1] |
| 48 | 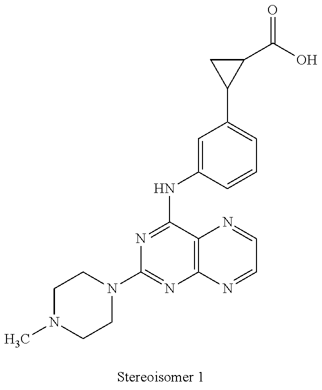 [SMILES: CN1CCN(c2nc(Nc3cccc(C4CC4C(=O)O)c3)c3nccnc3n2)CC1] |

In certain embodiments, the compound, or pharmaceutically acceptable salt, isotopically enriched analog, stereoisomer, mixture of stereoisomers, or prodrug thereof, is selected from Table 2.

TABLE 2
Structure

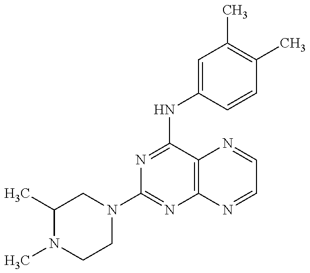

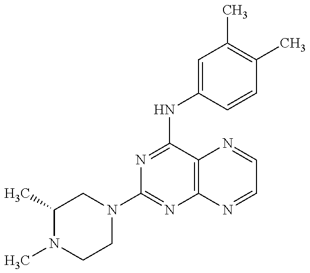

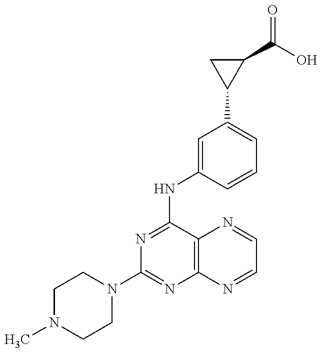 [SMILES: CN1CCN(c2nc(Nc3cccc([C@@H]4C[C@H]4C(=O)O)c3)c3nccnc3n2)CC1]

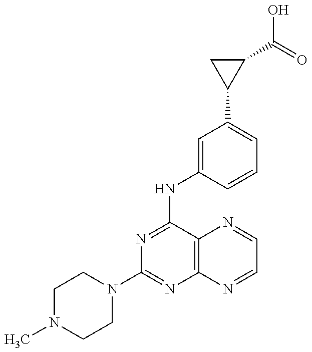 [SMILES: CN1CCN(c2nc(Nc3cccc([C@@H]4C[C@@H]4C(=O)O)c3)c3nccnc3n2)CC1]

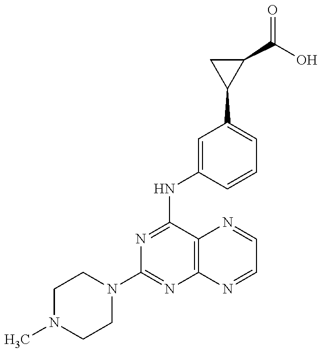 [SMILES: CN1CCN(c2nc(Nc3cccc([C@H]4C[C@H]4C(=O)O)c3)c3nccnc3n2)CC1]

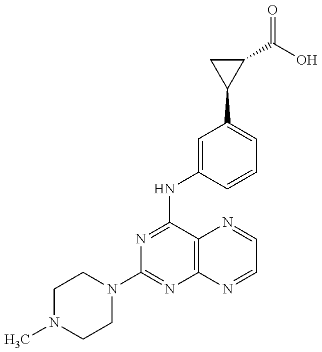 [SMILES: CN1CCN(c2nc(Nc3cccc([C@H]4C[C@@H]4C(=O)O)c3)c3nccnc3n2)CC1]

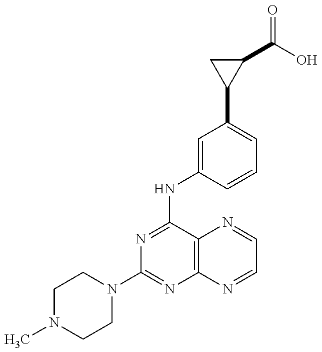

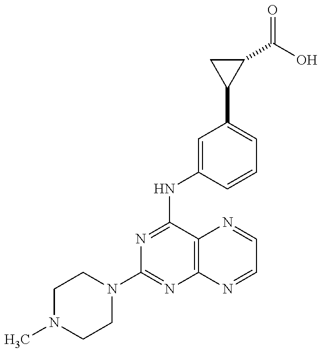

In certain embodiments, provided is a method for inhibiting the activity of MutSβ, comprising administering to a subject in need thereof, an effective amount of a compound disclosed herein, or a pharmaceutically acceptable salt, isotopically enriched analog, stereoisomer, mixture of stereoisomers, or prodrug thereof or a pharmaceutical composition as disclosed herein. In certain embodiments, provided is a method for inhibiting the activity of MutSβ, comprising administering to a subject in need thereof, an effective amount of a compound disclosed herein, or a pharmaceutically acceptable salt, isotopically enriched analog, stereoisomer, tautomer, or mixture of stereoisomers thereof or a pharmaceutical composition as disclosed herein. The inhibiting can be in vitro or in vivo.

In certain embodiments, provided is a compound as disclosed herein, or a pharmaceutically acceptable salt, isotopically enriched analog, stereoisomer, mixture of stereoisomers, or prodrug thereof, for use in inhibiting MutSβ activity (e.g., in vitro or in vivo).

In certain embodiments, the present disclosure provides use of a compound as disclosed herein, or a pharmaceutically acceptable salt, isotopically enriched analog, stereoisomer, mixture of stereoisomers, or prodrug thereof, in the manufacture of a medicament for inhibiting MutSβ activity (e.g., in vitro or in vivo).

In certain embodiments, provided is a method for treating a neurodegenerative or neurological disease or disorder, comprising administering to a subject in need thereof, an effective amount of a compound disclosed herein, or a pharmaceutically acceptable salt, isotopically enriched analog, stereoisomer, mixture of stereoisomers, or prodrug thereof or a pharmaceutical composition as disclosed herein. In certain embodiments, provided is a method for treating a neurodegenerative or neurological disease or disorder, comprising administering to a subject in need thereof, an effective amount of a compound disclosed herein, or a pharmaceutically acceptable salt, isotopically enriched analog, stereoisomer, tautomer, or mixture of stereoisomers thereof or a pharmaceutical composition as disclosed herein.

In certain embodiments, provided is a compound as disclosed herein, or a pharmaceutically acceptable salt, isotopically enriched analog, stereoisomer, mixture of stereoisomers, or prodrug thereof, for inhibiting trinucleotide repeat expansion promoted by MutSβ (see Guo et al., Cell Res., 2016, 26(7): 775-86). In certain embodiments, provided is a method of inhibiting trinucleotide repeat expansion promoted by MutSβ and/or treating a neurodegenerative or neurological disease or disorder in a subject in need thereof, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a disease or condition mediated, at least in part, by MutSβ, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a neurodegenerative or neurological disease or disorder associated with trinucleotide repeat expansion, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a neurodegenerative or neurological disease or condition mediated, at least in part, by MutSβ, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, the neurodegenerative or neurological disease or disorder associated with trinucleotide repeat expansion is Huntington's disease (HD), spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 8, spinocerebellar ataxia type 12, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, oculopharyngeal muscular dystrophy, or dentatorubro-pallidoluysian atrophy (see Brouwer et al, Bioessays, 2009, 31(1): 71-83).

In certain embodiments, provided is a compound disclosed herein, or a pharmaceutically acceptable salt, isotopically enriched analog, stereoisomer, mixture of stereoisomers, or prodrug thereof, or a pharmaceutical composition disclosed herein, for use in treating Huntington's disease (HD), spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 8, spinocerebellar ataxia type 12, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, oculopharyngeal muscular dystrophy, or dentatorubro-pallidoluysian atrophy.

In certain embodiments, provided is a compound disclosed herein, or a pharmaceutically acceptable salt, isotopically enriched analog, stereoisomer, tautomer, or mixture of stereoisomers thereof, or a pharmaceutical composition disclosed herein, for use in treating Huntington's disease (HD), spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 8, spinocerebellar ataxia type 12, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, oculopharyngeal muscular dystrophy, or dentatorubro-pallidoluysian atrophy.

In certain embodiments, the neurodegenerative or neurological disease or disorder is Huntington's disease (HD), spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 8, spinocerebellar ataxia type 12, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, oculopharyngeal muscular dystrophy, or dentatorubro-pallidoluysian atrophy. In certain embodiments, the neurodegenerative or neurological disease or disorder is Huntington's Disease (HD).

In certain embodiments, the present disclosure provides inhibitors of MutSβ for treatment of neurodegenerative or neurological diseases or disorders that are associated with trinucleotide repeat expansion. The present disclosure also provides methods of using inhibitors of MutSβ to treat, prevent or ameliorate neurodegenerative or neurological diseases or disorders.

In certain embodiments, the treating comprises reducing one or more symptoms or features of neurodegeneration.

In certain embodiments, one or more compounds or compositions as described herein is characterized that, when administered to a population of subjects, reduces one or more symptoms or features of neurodegeneration.

In certain embodiments, the present methods comprise administering an effective amount of a compound and/or composition as described herein (e.g., a compound of Formula I) to a subject in need thereof.

In certain embodiments, the subject is at risk of developing a neurodegenerative disorder. In certain embodiments, the subject is elderly. In certain embodiments, the subject is known to have a genetic risk factor for neurodegeneration. In certain embodiments, the subject has a family history of neurodegenerative disease. In certain embodiments, the subject expresses one or more copies of a known genetic risk factor for neurodegeneration. In certain embodiments, the subject is drawn from a population with a high incidence of neurodegeneration.

In certain embodiments, provided is a compound disclosed herein, or a pharmaceutically acceptable salt, isotopically enriched analog, stereoisomer, mixture of stereoisomers, or prodrug thereof, or a pharmaceutical composition described herein, for use in therapy. In certain embodiments, provided is a compound disclosed herein, or a pharmaceutically acceptable salt, isotopically enriched analog, stereoisomer, tautomer, or mixture of stereoisomers thereof, or a pharmaceutical composition described herein, for use in therapy.

### 4. Kits

Provided herein are also kits that include a compound of the disclosure, or a pharmaceutically acceptable salt, isotopically enriched analog, stereoisomer, mixture of stereoisomers, or prodrug thereof, and suitable packaging. In certain embodiments, a kit further includes instructions for use. In one aspect, a kit includes a compound of the disclosure, or a pharmaceutically acceptable salt, isotopically enriched analog, stereoisomer, mixture of stereoisomers, or prodrug thereof, and a label and/or instructions for use of the compounds in the treatment of the indications, including the diseases or conditions, described herein.

Provided herein are also articles of manufacture that include a compound described herein or a pharmaceutically acceptable salt, isotopically enriched analog, stereoisomer, mixture of stereoisomers, or prodrug thereof in a suitable container. The container may be a vial, jar, ampoule, preloaded syringe, or intravenous bag.

### 5. Pharmaceutical Compositions and Modes of Administration

Compounds provided herein are usually administered in the form of pharmaceutical compositions. Thus, provided herein are also pharmaceutical compositions that contain one or more of the compounds described herein, or a pharmaceutically acceptable salt, stereoisomer, mixture of stereoisomers, or prodrug thereof, and one or more pharmaceutically acceptable vehicles selected from carriers, adjuvants, and excipients. Suitable pharmaceutically acceptable vehicles may include, for example, inert solid diluents and fillers, diluents, including sterile aqueous solution and various organic solvents, permeation enhancers, solubilizers, and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, isotopically enriched analog, stereoisomer, mixture of stereoisomers, or prodrug thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include, e.g., lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, isotopically enriched analog, stereoisomer, mixture of stereoisomers, or prodrug thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, isotopically enriched analog, stereoisomer, mixture of stereoisomers, or prodrug thereof. When referring to these preformulation compositions as homogeneous, the active ingredient may be dispersed evenly throughout the composition so that the composition may be readily subdivided into equally effective unit dosage forms such as tablets, pills, and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In certain embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, in one embodiment, orally or nasally, from devices that deliver the formulation in an appropriate manner.

The amount of the compound in a pharmaceutical composition or formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below (“q.s.”=sufficient quantity).

### Formulation Example 1—Tablet Formulation

The following ingredients are mixed intimately and pressed into single scored tablets.

| Ingredient | Quantity per tablet, mg |
|---|---|
| compound of this disclosure | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

| Ingredient | Quantity per capsule, mg |
|---|---|
| compound of this disclosure | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| coloring | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compound of this disclosure | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this disclosure with Witepsol® H-15 (triglycerides of saturated vegetable fatty acid; Riches-Nelson, Inc., New York), and has the following composition:

| Ingredient | Amount |
|---|---|
| compound of this disclosure | 500 mg |
| Witepsol ® H-15 | balance |

### 6. Dosing

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In certain embodiments, about 0.1 and 100 mg/kg may be appropriate. In other embodiments, a dosage of between 0.5 and 60 mg/kg may be appropriate. In certain embodiments, a dosage of from about 0.0001 to about 100 mg per kg of body weight per day, from about 0.001 to about 50 mg of compound per kg of body weight, or from about 0.01 to about 10 mg of compound per kg of body weight may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

### 7. Synthesis of the Compounds

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents and starting materials may be purchased commercially, e.g., from Sigma Aldrich or other chemical suppliers.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups (“PG”) may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein. For example, protecting groups for alcohols, such as hydroxy, include silyl ethers (including trimethylsilyl (TMS), tert-butyldimethylsilyl (TBDMS), tri-iso-propylsilyloxymethyl (TOM), and triisopropylsilyl (TIPS) ethers), which can be removed by acid or fluoride ion, such as NaF, TBAF (tetra-n-butylammonium fluoride), HF-Py, or HF-NEt₃. Other protecting groups for alcohols include acetyl, removed by acid or base, benzoyl, removed by acid or base, benzyl, removed by hydrogenation, methoxyethoxymethyl ether, removed by acid, dimethoxytrityl, removed by acid, methoxymethyl ether, removed by acid, tetrahydropyranyl or tetrahydrofuranyl, removed by acid, and trityl, removed by acid. Examples of protecting groups for amines include carbobenzyloxy, removed by hydrogenolysis, p-methoxybenzyl carbonyl, removed by hydrogenolysis, tert-butyloxycarbonyl, removed by concentrated strong acid (such as HCl or CF₃COOH), or by heating to greater than about 80° C., 9-fluorenylmethyloxycarbonyl, removed by base, such as piperidine, acetyl, removed by treatment with a base, benzoyl, removed by treatment with a base, benzyl, removed by hydrogenolysis, carbamate group, removed by acid and mild heating, p-methoxybenzyl, removed by hydrogenolysis, 3,4-dimethoxybenzyl, removed by hydrogenolysis, p-methoxyphenyl, removed by ammonium cerium(IV) nitrate, tosyl, removed by concentrated acid (such as HBr or H₂SO₄) and strong reducing agents (sodium in liquid ammonia or sodium naphthalenide), troc (trichloroethyl chloroformate), removed by Zn insertion in the presence of acetic acid, and sulfonamides (Nosyl & Nps), removed by samarium iodide or tributyltin hydride.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### General Synthesis

Scheme I illustrates general methods which can be employed for the synthesis of compounds described herein, wherein n, m, p, q, L, X¹, R¹, R^{1a}, R², R^{2a}, R³, and R⁴ are independently as defined herein; LG is a leaving group (e.g., halo, alkoxy, etc); and Nuc is a nucleophilic group (e.g., amine, hydroxy, etc).

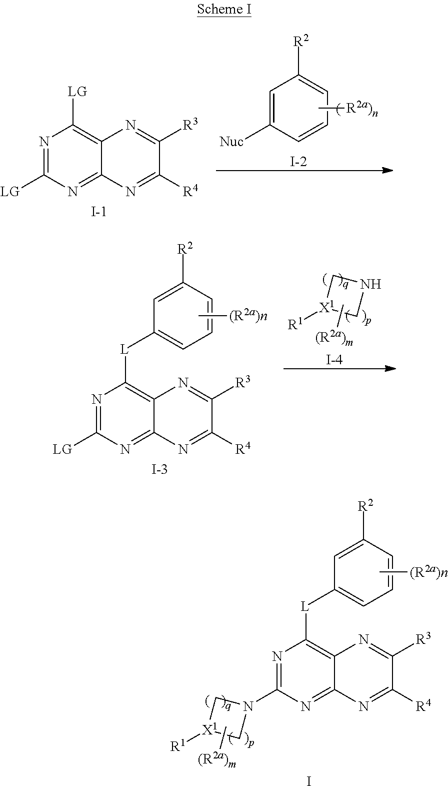

In Scheme I, compounds of Formula I-3 can be prepared by contacting compound I-1 with compound I-2 under suitable reaction conditions (e.g., in the presence of a base such as DIPEA), followed by optional functionalization or deprotection when required. Compounds of Formula I can then be prepared by contacting compound I-3 with compound I-4 under suitable acidic nucleophilic aromatic substitution conditions (e.g., in the presence of trifluoroacetic acid), followed by optional functionalization or deprotection when required. Alternatively, compounds of Formula I can be prepared by contacting compound I-3 with compound I-4 under suitable basic nucleophilic aromatic substitution conditions (e.g., in the presence of a base such as DIPEA), followed by optional functionalization or deprotection when required.

Scheme II also illustrates general methods which can be employed for the synthesis of compounds described herein, wherein n, m, p, q, L, X¹, R¹, R^{1a}, R², R^{2a}, R³, and R⁴ are independently as defined herein; and Nuc is a nucleophilic group (e.g., amine, hydroxy, etc).

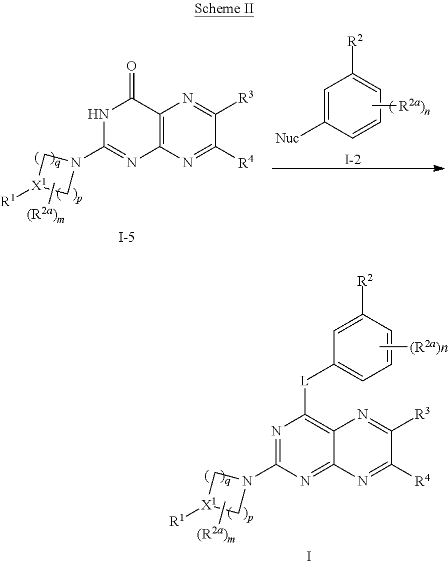

In Scheme II, compounds of Formula I can be prepared by contacting compound I-5 with compound I-2 under suitable reaction conditions (e.g., in the presence of a suitable base and coupling reagent), followed by optional functionalization or deprotection when required. In some embodiments, Nuc is an —NH₂, and L is —NH—.

Upon each reaction completion, each of the intermediate or final compounds can be recovered, and optionally purified, by conventional techniques such as neutralization, extraction, precipitation, chromatography, filtration and the like.

It should be understood that any of the compounds or intermediates shown in Scheme I or Scheme II may be prepared using traditional methods or purchased from commercial sources. In addition, any of the intermediates or any product obtained by the process outlined in Scheme I or Scheme II can be derivatized at any step to provide various compounds of Formula I. In certain embodiments, the various substituents of the compounds or intermediates as used in Scheme I and Scheme II are as defined for Formula I.

In certain embodiments, provided is a process for providing a compound of Formula I, comprising contacting a compound of Formula I-3:

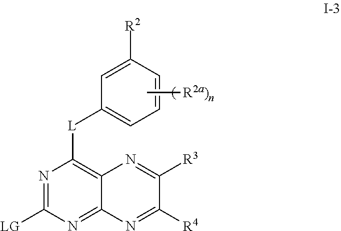

with a compound of Formula I-4:

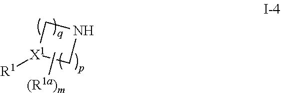

under conditions sufficient to provide a compound of Formula I, wherein n, m, p, q, L, X¹, R¹, R^{1a}, R², R^{2a}, R³, and R⁴ are independently as defined herein; and LG is a leaving group (e.g., halo, alkoxy, etc).

In certain embodiments, the LG is halo.

In certain embodiments, provided is a process for providing a compound of Formula I, comprising contacting a compound of Formula I-5:

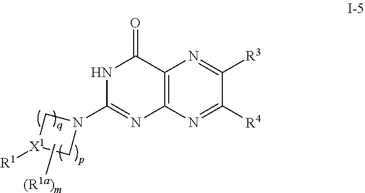

with a compound of Formula I-2:

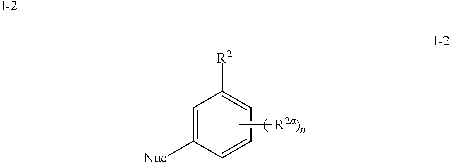

under conditions sufficient to provide a compound of Formula I, wherein n, m, p, q, L, X¹, R¹, R^{1a}, R², R^{2a}, R³, and R⁴ are independently as defined herein; and Nuc is a nucleophilic group (e.g., amine, hydroxy, etc).

In certain embodiments, Nuc is an —NH₂, and L is —NH—.

### EXAMPLES

The following examples are included to demonstrate specific embodiments of the disclosure. It should be appreciated by those skilled in the art that the techniques disclosed in the examples which follow represent techniques to function well in the practice of the disclosure, and thus can be considered to constitute specific modes of its practice. However, those of skill in the art should, in light of the present disclosure, appreciate that many changes can be made in the specific embodiments which are disclosed and still obtain a like or similar result without departing from the spirit and scope of the disclosure.

### General Experimental Methods

Commercially available reagents and solvents (HPLC grade) were used without further purification.

NMR Spectroscopy: ¹H NMR spectra were recorded on a Bruker Avance III HD 500 MHz spectrometer or Bruker Avance III HD 400 MHz spectrometer in deuterated solvents. Chemical shifts (δ) are in parts per million. Thin-layer chromatography (TLC) analysis was performed with Kieselgel 60 F254 (Merck) plates and visualized using UV light. Variable temperature NMR's (where run) were used to resolve rotomeric spectra.

Analytical HPLC-MS (METCR1603), was performed on Hewlett Packard HPLC systems using reverse phase Phenomenex Gemini C18 columns (2 μm, 2.0×100 mm), gradient 5-100% B (A=2 mM ammonium bicarbonate in water buffered to pH 10, B=acetonitrile) over 5.9 min injection volume 3 μL, flow=0.5 mL/min. UV spectra were recorded at 215 nm using a Waters photo diode array detector. Mass spectra were obtained over the range m/z 150 to 850 at a sampling rate of 2 scans per second using a Waters ZQ. Data were integrated and reported using OpenLynx software.

Analytical HPLC-MS (METCR1278) was performed on Shimadzu LCMS-2010EV systems using reverse phase Atlantis dC18 columns (3 μm, 2.1×50 mm), gradient 5-100% B (A=water/0.1% formic acid, B=acetonitrile/0.1% formic acid) over 3 min, injection volume 3 μL, flow=1.0 mL/min. UV spectra were recorded at 215 nm using a Waters 2788 dual wavelength UV detector. Mass spectra were obtained over the range m/z 150 to 850 at a sampling rate of 2 scans per second using Waters LCT or analytical HPLC-MS on Shimadzu LCMS-2010EV systems using reverse phase Waters Atlantis dC18 columns (3 μm, 2.1×100 mm), gradient 5-100% B (A=water/0.1% formic acid, B=acetonitrile/0.1% formic acid) over 7 min, injection volume 3 μL, flow=0.6 mL/min. UV spectra were recorded at 215 nm using a Waters 2996 photo diode array. Data were integrated and reported using Shimadzu PsiPort software.

Analytical HPLC-MS (METCR1410), was performed on Shimadzu LCMS-2010EV systems using reverse phase Kinetex Core-Shell C18 50×2.1 mm 5 μm column, gradient 5-100% B (A=water/0.1% formic acid, B=MeCN/0.1% formic acid) over 1.2 min injection volume 3 μL, flow=1.2 mL/min. UV spectra were recorded at 215 nm using a SPD-M20A photo diode array detector. Mass spectra were obtained over the range m/z 100 to 1000 at a sampling rate of 2 scans per second using a LCMS2010EV. Data were integrated and reported using Shimadzu LCMS-Solutions and PsiPort software.

Analytical HPLC-MS (METCR1704) was performed on a Waters Acquity UPLC system with Waters PDA and ELS detectors using a Waters Acquity UPLC BEH C-18 column, (1.7 μM, 2.1 mm×50 mm at a column temp of 40° C., gradient 5-100% B (A=water/0.1% formic acid; B=MeCN/0.1% formic acid) over 1.1 min, then 100% B for 0.25 min, flow=0.9 mL/min.

Analytical HPLC-MS (METCR1906) was performed on a Waters Acquity UPLC system with Waters PDA and ELS detectors using a Waters Acquity UPLC CORTECS C-8 column, (1.6 μM, 2.1 mm×50 mm at a column temp of 40° C., gradient 5-100% B (A=water/0.1% formic acid; B=MeCN/0.10% formic acid) over 1.1 min, then 100% B for 0.3 min, flow=0.9 mL/min.

Analytical HPLC-MS (MET-uHPLC-AB-2005) was performed on a Waters Acquity UPLC system with Waters PDA and ELS detectors using a Waters UPLC BEH C-18 column, (1.7 μM, 2.1 mm×30 mm) at a column temp of 40° C., gradient 1-100% B (A=2 nM ammonium bicarbonate modified to pH10 with ammonium hydroxide; B=MeCN) over 1.1 min, then 100% B for 0.25 min, flow=1.0 mL/min.

Analytical HPLC-MS (MET-uHPLC-AB2010) was performed on a Waters Acquity UPLC system with Waters PDA and ELS detectors using a Waters UPLC BEH C-18 column, (1.7 μM, 2.1 mm×30 mm) at a column temp of 55° C., gradient 1-100% B (A=2 nM ammonium bicarbonate modified to pH10 with ammonium hydroxide; B=MeCN) over 1.1 min, then 100% B for 0.25 min, flow=1.0 mL/min.

Alternatively, HPLC-MS (MET-uHPLC-AB-101) analytical HPLC-MS were performed on a Waters Acquity UPLC system with Waters PDA and ELS detectors using a Phenomenex Kinetex-XB C-18 column, (1.7 μM, 2.1 mm×100 mm) at a column temp of 40° C., gradient 5-100% B (A=water/0.10% formic acid; B=MeCN/0.10% formic acid) over 5.3 min, then 100% B for 0.5 min, flow=0.6 mL/min.

Alternatively, HPLC-MS (MET-CR-AB106) analytical HPLC-MS were performed on a Waters Acquity UPLC system with Waters PDA and ELS detectors using a Waters Acquity UPLC CORTECS C-8 column, (1.6 μM, 2.1 mm×50 mm at a column temp of 40° C., gradient 5-100% B (A=water/0.10% formic acid; B=MeCN/0.10% formic acid) over 5.3 min, then 100% B for 0.5 min, flow=0.6 mL/min.

Alternatively, HPLC-MS (MET-uHPLC-AB-107) was performed on a Waters Acquity UPLC system with Waters PDA and ELS detectors using a Waters UPLC BEH C-18 column, (1.7 μM, 2.1 mm×100 mm) at a column temp of 55° C., gradient 5-100% B (A=2 nM ammonium bicarbonate modified to pH10 with ammonium hydroxide; B=MeCN) over 5.3 min, then 100% B for 0.5 min, flow=0.6 mL/min.

Alternatively, the following analytical methods were used:

### Acidic QC Methods:

### AcHSSC18—Standard Acidic UPLC-MS

| Instrumentation | UPLC + Waters DAD + Waters SQD2, single | | |
|---|---|---|---|
| | quadrapole UPLC-MS | | |
| MassLynx Files | | | |
| Column | Acquity UPLC HSS C18 1.8 μm 100 × 2.1 mm. | | |
| | (Plus guard cartridge), maintained at temp | | |
| Mobile Phase A | Water (High purity via PureLab Option unit) with | | |
| | 0.1% formic acid | | |
| Mobile Phase B | Acetonitrile (Far UV grade) with 0.1% (V/V) | | |
| | formic acid | | |
| Flow | 0.4 mL/min | | |
| Gradient Program | Time (mins) | % A | % B |
| | 0.0 | 95 | 05 |
| | 0.4 | 95 | 05 |
| | 6.0 | 05 | 95 |
| | 6.8 | 05 | 95 |
| | 7.0 | 95 | 05 |
| | 8.0 | 95 | 05 |

| | |
|---|---|
| Sample | 0.5-2 μL (concentration ~0.2-1 mg/mL). |
| Detectors | UV, diode array 210 nm-400 nm Resolution 1.2 nm |
| | Other wavelength traces are extracted from the |
| | DAD data |
| | MS, mass 100-700 (or −1500 for HM method) in ES+ |
| | & ES− (300 μL/min split to MS) |

10 cm_Formic_AQ—Standard Acidic UPLC-MS

| Instrumentation | UPLC + Waters DAD + Waters SQD2, single | | |
|---|---|---|---|
| | quadrapole UPLC-MS | | |
| MassLynx Files | | | |
| Column | Acquity UPLC HSS C18 1.8 μm 100 × 2.1 mm. | | |
| | (Plus guard cartridge), maintained at temp | | |
| Mobile Phase A | Water (High purity via PureLab Option unit) with | | |
| | 0.1% formic acid | | |
| Mobile Phase B | Acetonitrile (Far UV grade) with 0.1% (V/V) | | |
| | formic acid | | |
| Flow | 0.5 mL/min | | |
| Gradient Program | Time (mins) | % A | % B |
| | 0.0 | 95 | 05 |
| | 1.2 | 95 | 05 |
| | 3.5 | 0 | 100 |
| | 4.9 | 0 | 100 |
| | 5 | 95 | 05 |
| | 6 | 95 | 05 |

| | |
|---|---|
| Sample | 0.5-2 μL (concentration ~0.2-1 mg/mL). |
| Detectors | UV, diode array 210 nm-400 nm Resolution 1.2 nm |
| | Other wavelength traces are extracted from the |
| | DAD data |
| | MS, mass 100-700 (or −1500 for HM method) in ES+ |
| | & ES− (300 μL/min split to MS) |

### Basic QC Methods

### BicarbBEHC18—Standard Basic UPLC-MS

| Instrumentation | UPLC + Waters DAD + Waters SQD2, single | | |
|---|---|---|---|
| | quadrapole UPLC-MS | | |
| MassLynx Files | | | |
| Column | Acquity UPLC BEH Shield RP18 1.7 μm 100 × 2.1 | | |
| | mm. (Plus guard cartridge), maintained at temp | | |
| Mobile Phase A | Water (High purity via PureLab Option unit) with 10 | | |
| | mM ammonium bicarbonate (ammonium hydrogen | | |
| | carbonate) | | |
| Mobile Phase B | Acetonitrile (Far UV grade) | | |
| Flow | 0.4 mL/min | | |
| Gradient Program | Time (mins) | % A | % B |
| | 0.0 | 95 | 05 |
| | 0.4 | 95 | 05 |
| | 6.0 | 05 | 95 |
| | 6.8 | 05 | 95 |
| | 7.0 | 95 | 05 |
| | 8.0 | 95 | 05 |

| | |
|---|---|
| Sample | 0.5-2 μL (concentration ~0.2-1 mg/mL). |
| Detectors | UV, diode array 210 nm-400 nm Resolution 1.2 nm |
| | Other wavelength traces are extracted from the |
| | DAD data |
| | MS, mass 100-700 (or −1500 for HM method) in ES+ |
| | & ES− (300 μL/min split to MS) |

10 cm_Bicarb_AQ−Standard Basic UPLC-MS

| Instrumentation | UPLC + Waters DAD + Waters SQD2, single | | |
|---|---|---|---|
| | quadrapole UPLC-MS | | |
| MassLynx Files | | | |
| Column | Acquity UPLC BEH Shield RP18 1.7 μm 100 × 2.1 | | |
| | mm. (Plus guard cartridge), maintained at temp | | |
| Mobile Phase A | Water (High purity via PureLab Option unit) with 10 | | |
| | mM ammonium bicarbonate (ammonium hydrogen | | |
| | carbonate) | | |
| Mobile Phase B | Acetonitrile (Far UV grade) | | |
| Flow | 0.5 mL/min | | |
| Gradient Program | Time (mins) | % A | % B |
| | 0.0 | 95 | 05 |
| | 1.2 | 95 | 05 |
| | 3.5 | 0 | 100 |
| | 4.9 | 0 | 100 |
| | 5 | 95 | 05 |
| | 6 | 95 | 05 |

| | |
|---|---|
| Sample | 0.5-2 μL (concentration ~0.2-1 mg/mL). |
| Detectors | UV, diode array 210 nm-400 nm Resolution 1.2 nm |
| | Other wavelength traces are extracted from the |
| | DAD data |
| | MS, mass 100-700 (or −1500 for HM method) in ES+ |
| | & ES− (300 μL/min split to MS) |

Final compounds display purity of >95% or >93% as determined by methods described herein, unless stated otherwise.

### General Synthetic Procedures

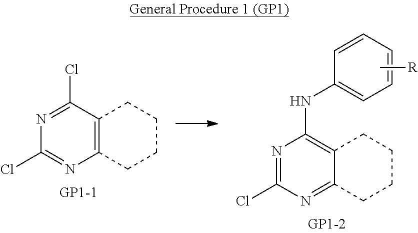

DIPEA (1.8 eq.) was added to a stirred suspension of GP1-1 (1.0 eq.) and aniline (1.1 eq.) in IPA (1 M) and the mixture was heated at reflux (90° C. external) for 2 hours. The mixture was allowed to cool to RT and the product collected by filtration. Alternatively, the reaction mixture was concentrated in vacuo to afford the crude product. The product was separated by FCC to afford the desired product (GP1-2) and the material was used as such in the next stage.

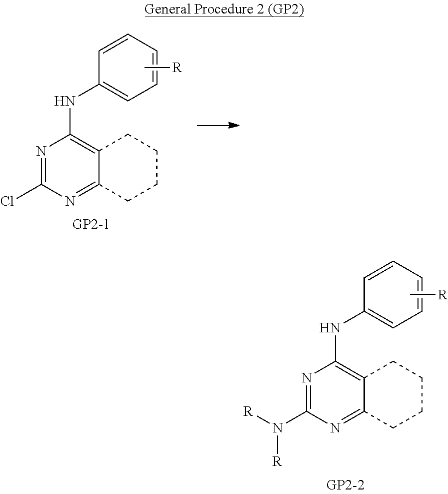

Trifluoroacetic acid (2.0 eq.) was added to a stirred mixture of GP2-1 (1.0 eq.) and amine (2.0 eq.) in 1-butanol (0.2 M) and the reaction sealed and heated at 120° C. for 2 hours. After allowing to cool to RT, the mixture was diluted with dichloromethane (20 mL) and quenched with saturated aqueous sodium carbonate solution (10 mL). The biphasic mixture was separated using a hydrophobic frit and the aqueous layer was extracted with dichloromethane (2×10 mL). The organic filtrate was concentrated in vacuo to afford the crude product. The product was separated by high pH prep. HPLC (ammonium hydroxide-water-acetonitrile) or low pH prep. HPLC (formic acid-water-acetonitrile) to afford the title compound after freeze drying.

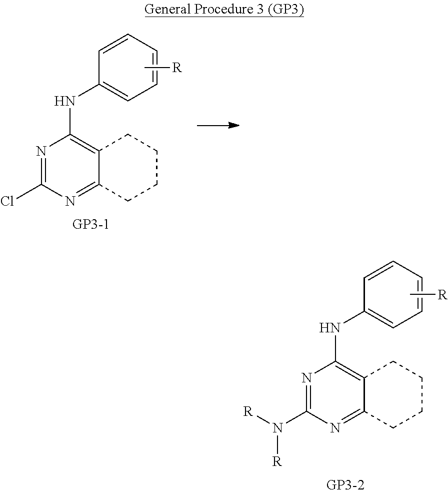

DIPEA (1.8 eq.) was added to a stirred mixture of GP3-1 (1.0 eq.) and amine (1.0 eq.) in DMSO (0.18 M) and the reaction sealed and heated at 100° C. for 2 hours. After allowing to cool to RT, the mixture was diluted with water (5 mL) to afford precipitation. Alternatively, the mixture was diluted with water (10 mL), the pH adjusted to 14 with 2 M aqueous sodium hydroxide and the product extracted into DCM (3×15 mL) using a hydrophobic frit to separate the phases. The organic filtrate was concentrated in vacuo to afford the crude product. Where required, the product was separated by high pH prep. HPLC (ammonium hydroxide-water-acetonitrile), low pH prep. HPLC (formic acid-water-acetonitrile), or reverse phase column chromatography (ammonium hydroxide-water-acetonitrile) to afford the title compound after freeze drying.

### Example 1

### Preparation of N-(3,4-dimethylphenyl)-2-(4-methylpiperazin-1-yl)pteridin-4-amine (Compound 2)

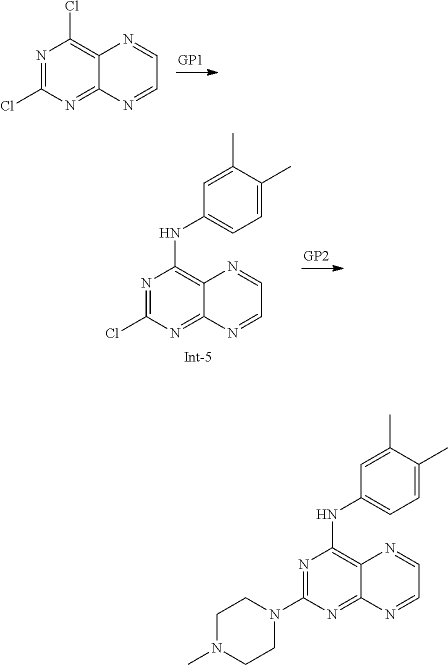

### Step 1: Preparation of 2-chloro-N-(3,4-dimethylphenyl)pteridin-4-amine (Int-5)

Prepared according to GP1, using DIPEA (0.31 mL, 1.79 mmol), 2,4-dichloropteridine (200 mg, 1.00 mmol) and 3,4-dimethylaniline (120 mg, 1.00 mmol) in IPA (3 mL). The product was separated by filtration, washing with methanol (3 mL) to afford Int-5 (203 mg, 68%). The material was used as such in the next stage. ¹H NMR (400 MHz, DMSO) δ 10.84 (s, 1H), 9.14 (d, J=2.0 Hz, 1H), 8.93 (d, J=2.0 Hz, 1H), 7.63 (d, J=9.3 Hz, 2H), 7.19 (d, J=8.0 Hz, 1H), 2.25 (s, 3H), 2.23 (s, 3H). Tr(METCR1704)=0.87 min, m/z (ES⁺) [M+H]⁺=286.0, 288.0 (Cl³⁵/Cl³⁷), 97%.

### Step 2: Preparation of N-(3,4-dimethylphenyl)-2-(4-methylpiperazin-1-yl)pteridin-4-amine (Compound 2)

Prepared according to GP2, using trifluoroacetic acid (27 μL, 0.35 mmol), 2-chloro-N-(3,4-dimethylphenyl)pteridin-4-amine (Int-5, 100%, 50 mg, 0.18 mmol) and 1-methylpiperazine (40 μL, 0.18 mmol) in 1-butanol (1 mL). The product was separated by reverse phase FCC (Biotage isolera, high pH, 12 g C18 column, 10 to 100% acetonitrile in water) to afford Compound 2 (41 mg, 64%) after freeze drying.

¹H NMR (400 MHz, DMSO) δ 9.99 (s, 1H), 8.76 (d, J=2.1 Hz, 1H), 8.39 (d, J=2.1 Hz, 1H), 7.75 (d, J=1.9 Hz, 1H), 7.59 (dd, J=8.1, 2.2 Hz, 1H), 7.12 (d, J=8.3 Hz, 1H), 3.85 (s, 4H), 2.40 (s, 4H), 2.24-2.17 (m, 9H). Tr(MET-uHPLC-AB-101)=2.04 min, m/z (ES⁺) [M+H]⁺

### Example 2

### Preparation of N-(4-Chlorophenyl)-2-(4-ethylpiperazin-1-yl)pteridin-4-amine (Compound 4)

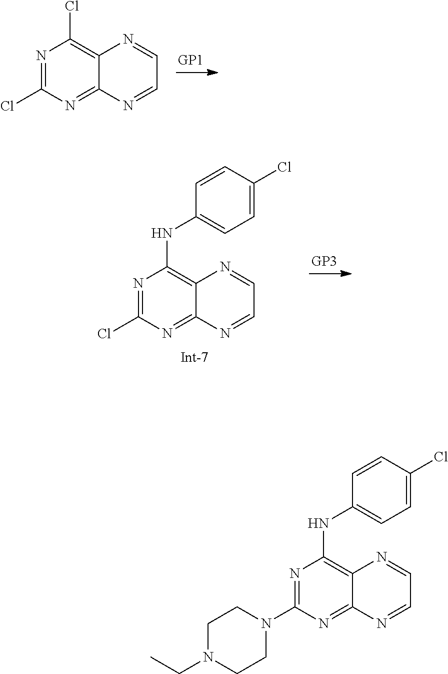 [SMILES: Clc1nc(Cl)c2nccnc2n1 ; Clc1ccc(Nc2nc(Cl)nc3nccnc23)cc1 ; CCN1CCN(c2nc(Nc3ccc(Cl)cc3)c3nccnc3n2)CC1]

### Step 1: Preparation of 2-chloro-N-(4-chlorophenyl)pteridin-4-amine (Int-7)

Prepared according to GP1, using DIPEA (0.14 mL, 0.85 mmol), 2,4-dichloropteridine (100 mg, 0.47 mmol) and 4-chloroaniline (60 mg, 0.47 mmol) in IPA (1 mL). The product was separated by filtration, washing with water (3 mL) and IPA (3 mL) to afford Int-7 (105 mg, 74%). The material was used as such in the next stage.

¹H NMR (400 MHz, DMSO) δ 11.15 (s, 1H), 9.17 (d, J=2.0 Hz, 1H), 8.97 (d, J=2.0 Hz, 1H), 8.02-7.94 (m, 2H), 7.56-7.47 (m, 2H). Tr(METCR1704)=0.85 min, m/z (ES⁺) [M+H]⁺=292.0, 293.9 (Cl³⁵/Cl³⁷), 97%.

### Step 2: Preparation of N-(4-chlorophenyl)-2-(4-ethylpiperazin-1-yl)pteridin-4-amine (Compound 4)

Prepared according to GP3, using DIPEA (0.11 mL, 0.62 mmol), 2-chloro-N-(4-chlorophenyl)pteridin-4-amine (Int-7, 97%, 100 mg, 0.34 mmol) and 1-ethylpiperazine (39 mg, 0.34 mmol) in DMSO (2.5 mL). Water was added and the precipitate that formed was filtered, washing with DMSO (5 mL) and water (5 mL) to afford Compound 4.

¹H NMR (400 MHz, DMSO) δ 10.34 (s, 1H), 8.81 (d, J=2.1 Hz, 1H), 8.43 (d, J=2.0 Hz, 1H), 8.02-7.94 (m, 2H), 7.49-7.41 (m, 2H), 3.87-3.83 (m, 4H), 2.48-2.40 (m, 4H), 2.37 (q, J=7.2 Hz, 2H), 1.04 (t, J=7.2 Hz, 3H). Tr(MET-uHPLC-AB-101)=1.86 min, m/z (ES⁺) [M+H]⁺ 370.3, 372.3 (Cl³⁵/Cl³⁷) 98%.

### Example 3

### Preparation of: 2-(4-methylpiperazin-1-yl)-N-(m-tolyl)pteridin-4-amine (Compound 6)

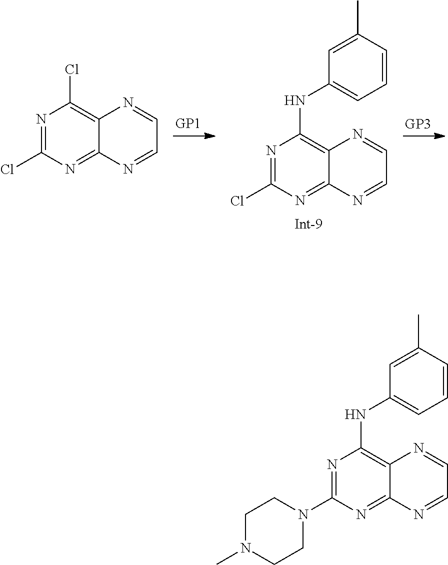 [SMILES: Clc1nc(Cl)c2nccnc2n1 ; Cc1cccc(Nc2nc(Cl)nc3nccnc23)c1 ; Cc1cccc(Nc2nc(N3CCN(C)CC3)nc3nccnc23)c1]

### Step 1: Preparation of 2-chloro-N-(m-tolyl)pteridin-4-amine (Int-9)

Prepared according to GP1, using DIPEA (78 μL, 0.44 mmol), 2,4-dichloropteridine (50 mg, 0.25 mmol) and 3-methylaniline (27 mg, 0.25 mmol) in IPA (0.75 mL). The product was separated by reverse phase FCC (Biotage isolera, low pH, 12 g C18 column, 10 to 100% acetonitrile in water) to afford Int-9 (45 mg, 63%) after freeze drying. The material was used as such in the next stage.

¹H NMR (400 MHz, DMSO) δ 10.90 (s, 1H), 9.16 (d, J=2.0 Hz, 1H), 8.95 (d, J=2.0 Hz, 1H), 7.81-7.70 (m, 2H), 7.37-7.28 (m, 1H), 7.05 (d, J=7.8 Hz, 1H), 2.35 (s, 3H). Tr(METCR1704)=0.81 min, m/z (ES⁺) [M+H]⁺=272.0, 274.0 (Cl³⁵/Cl³⁷), 95%.

### Step 2: Preparation of 2-(4-methylpiperazin-1-yl)-N-(m-tolyl)pteridin-4-amine (Compound 6)

Prepared according to GP3, using DIPEA (46 μL, 0.27 mmol), 2-chloro-N-(m-tolyl)pteridin-4-amineamine (Int-9, 95%, 40 mg, 0.15 mmol) and 1-methylpiperazine (15 mg, 0.15 mmol) in DMSO (1 mL). Water was added and the precipitate that formed over 18 h was filtered, washing with water (5 mL) to afford Compound 6.

¹H NMR (500 MHz, DMSO) δ 10.07 (s, 1H), 8.79 (d, J=2.0 Hz, 1H), 8.42 (d, J=2.0 Hz, 1H), 7.84 (s, 1H), 7.70 (d, J=8.2 Hz, 1H), 7.27 (app. t, J=7.8 Hz, 1H), 6.94 (d, J=7.5 Hz, 1H), 3.89-3.84 (m, 4H), 2.43-2.37 (m, 4H), 2.32 (s, 3H), 2.22 (s, 3H). Tr(MET-uHPLC-AB-101)=1.59 min, m/z (ES⁺) [M+H]⁺ 336.2, 100%.

### Example 4

### Preparation of N-(3-chloro-4-methyl-phenyl)-2-(4-methylpiperazin-1-yl)pteridin-4-amine (Compound 20)

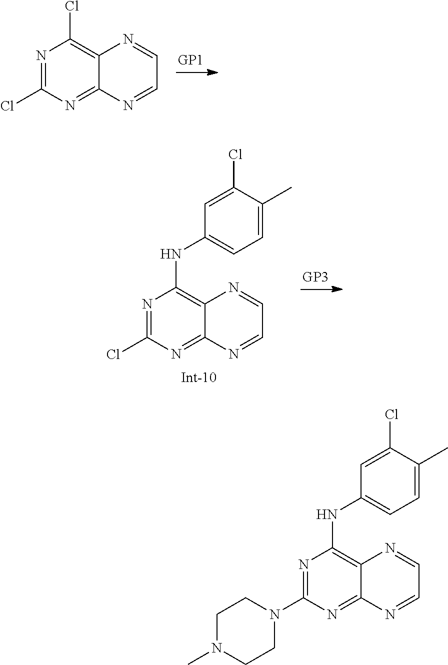

### Step 1: Preparation of 2-chloro-N-(3-chloro-4-methyl-phenyl)pteridin-4-amine (Int-10)

Prepared according to GP1, using DIPEA (78 μL, 0.45 mmol), 2,4-dichloropteridine (50 mg, 0.25 mmol) and 3-chloro-4-methylaniline (35 mg, 0.249 mmol) in IPA (0.75 mL) and the reaction mixture was concentrated in vacuo to afford the crude product. The product was separated by FCC (Biotage isolera, 25 g Sfar Duo column, eluent: 5 to 80% ethyl acetate in heptane) to afford Int-10 (25 mg, 22%). [M+H]⁺ 306.0/308.0/310.0 (Cl³⁵Cl³⁵/Cl³⁵—Cl³⁷/Cl³⁷—Cl³⁷).

### Step 2: Preparation of N-(3-chloro-4-methyl-phenyl)-2-(4-methylpiperazin-1-yl)pteridin-4-amine (Compound 20)

Prepared according to GP3, using DIPEA (17 μL, 0.098 mmol), 2-chloro-N-(3-chloro-4-methyl-phenyl)pteridin-4-amine (Int-10, 66%, 25 mg, 0.054 mmol) and 1-methylpiperazine (7.2 μL, 0.065 mmol) in anhydrous DMSO (0.5 mL) and the products were extracted into DCM from aqueous NaOH (pH 14). The products were separated by high pH prep. HPLC (ammonium hydroxide-water-acetonitrile) to afford Compound 20.

Compound 20 after freeze drying. ¹H NMR (500 MHz, DMSO) δ 10.33 (s, 1H), 8.81 (d, J=2.1 Hz, 1H), 8.44 (d, J=2.0 Hz, 1H), 8.17 (d, J=2.2 Hz, 1H), 7.78 (dd, J=8.3, 2.2 Hz, 1H), 7.35 (dd, J=8.3, 0.8 Hz, 1H), 3.87 (app. t, J=5.1 Hz, 4H), 2.40 (app. t, J=5.1 Hz, 4H), 2.32 (s, 3H), 2.23 (s, 3H). Tr(MET-uHPLC-AB-107)=3.62 min, m/z (ES⁺) [M+H]⁺ 370.2/372.2 (Cl³⁵/Cl³⁷), 100%.

### Example 5

### Preparation of 2-(4-ethylpiperazin-1-yl)-N-(4-fluoro-3-methyl-phenyl)pteridin-4-amine (Compound 3)

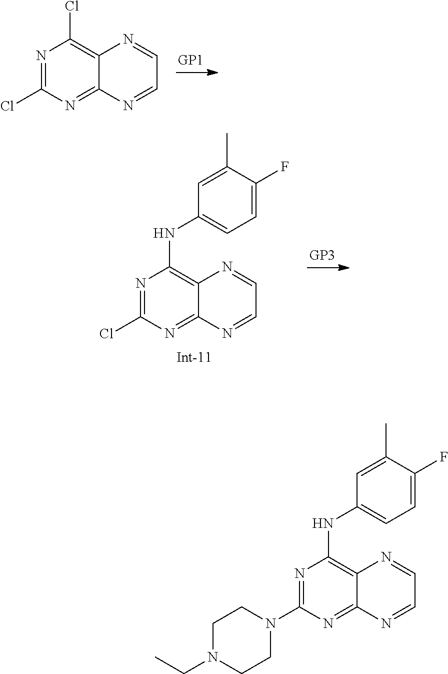 [SMILES: Clc1nc(Cl)c2nccnc2n1 ; Cc1cc(Nc2nc(Cl)nc3nccnc23)ccc1F ; CCN1CCN(c2nc(Nc3ccc(F)c(C)c3)c3nccnc3n2)CC1]

### Step 1: Preparation of 2-chloro-N-(4-fluoro-3-methyl-phenyl)pteridin-4-amine (Int-11)

Prepared according to GP1, using DIPEA (78 μL, 0.45 mmol), 2,4-dichloropteridine (50 mg, 0.25 mmol) and 4-fluoro-3-methylaniline (31 mg, 0.249 mmol) in IPA (0.75 mL) and the reaction mixture was concentrated in vacuo to afford the crude product. The product was separated by FCC (Biotage isolera, 25 g Sfar Duo column, eluent: 5 to 80% ethyl acetate in heptane) to afford Int-11 (54.7 mg, 71%). ¹H NMR (400 MHz, DMSO) δ 10.99 (s, 1H), 9.15 (d, J=2.0 Hz, 1H), 8.94 (d, J=2.0 Hz, 1H), 7.84-7.65 (m, 2H), 7.22 ((dd, ³J_{HF}=9.2 Hz, J=9.2 Hz, 1H), 2.27 (d, ⁴J_{HF}=2.0 Hz, 3H). ¹⁹F NMR (376 MHz, DMSO) δ −121.17. Tr(MET-uPLC-AB2010)=0.70 min, m/z (ES⁺) [M+H]⁺ 290.0/292.0 (Cl³⁵/Cl³⁷), 93%.

### Step 2: Preparation of 2-(4-ethylpiperazin-1-yl)-N-(4-fluoro-3-methyl-phenyl)pteridin-4-amine (Compound 3)

Prepared according to GP3, using DIPEA (55 μL, 0.32 mmol), 2-chloro-N-(4-fluoro-3-methyl-phenyl)pteridin-4-amine (Int-11, 93%, 55 mg, 0.18 mmol) and 1-ethylpiperazine (27 μL, 0.21 mmol) in anhydrous DMSO (1.7 mL) and the products were extracted into DCM from aqueous NaOH (pH 14). The product was separated by high pH prep. HPLC (ammonium hydroxide-water-acetonitrile) to afford Compound 3 after freeze drying. ¹H NMR (500 MHz, DMSO) δ 10.17 (s, 1H), 8.79 (d, J=2.0 Hz, 1H), 8.41 (d, J=2.0 Hz, 1H), 7.89 (dd, ⁴J_{HF}=7.0, J=2.5 Hz, 1H), 7.72 (ddd, J=9.0, ⁴J_{HF}=4.6, J=2.8 Hz, 1H), 7.16 (dd, ³J_{HF}=9.2, J=9.2 Hz, 1H), 3.85 (br. s, 4H), 2.44 (app. t, J=5.1 Hz, 4H), 2.36 (q, J=7.2 Hz, 2H), 2.25 (d, ⁴J_{HF}=2.0 Hz, 3H), 1.04 (t, J=7.2 Hz, 3H). ¹⁹F NMR (376 MHz, DMSO) δ −123.09. Tr(MET-uHPLC-AB-107)=3.55 min, m/z (ES⁺) [M+H]⁺ 386.3, 100%.

### Example 6

### Preparation of N-(3,4-difluorophenyl)-2-(4-ethylpiperazin-1-yl)pteridin-4-amine (Compound 16)

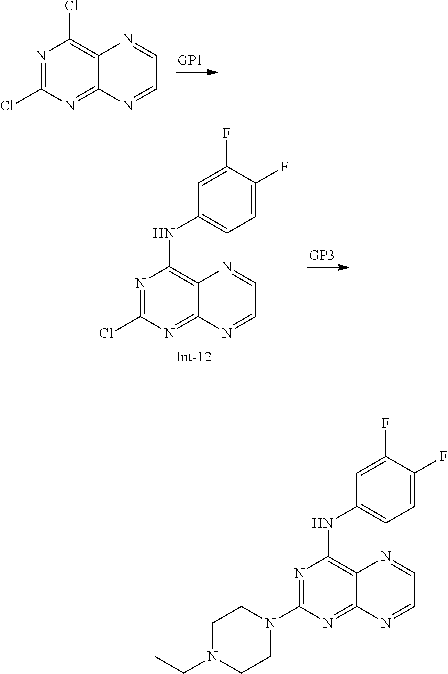 [SMILES: Clc1nc(Cl)c2nccnc2n1 ; Fc1ccc(Nc2nc(Cl)nc3nccnc23)cc1F ; CCN1CCN(c2nc(Nc3ccc(F)c(F)c3)c3nccnc3n2)CC1]

### Step 1: Preparation of 2-chloro-N-(3,4-difluorophenyl)pteridin-4-amine (Int-12)

Prepared according to GP1, using DIPEA (78 μL, 0.45 mmol), 2,4-dichloropteridine (50 mg, 0.25 mmol) and 3,4-difluoroaniline (32 mg, 0.25 mmol) in IPA (0.75 mL) and the reaction mixture was concentrated in vacuo to afford the crude product. The product was separated by FCC (Biotage isolera, 25 g Sfar Duo column, eluent: 5 to 100% ethyl acetate in heptane) to afford Int-12 (43 mg, 55%).

¹H NMR (400 MHz, DMSO) δ 11.20 (s, 1H), 9.18 (d, J=2.0 Hz, 1H), 8.98 (d, J=2.0 Hz, 1H), 8.10 (ddd, ³J_{HF}=13.1, ⁴J_{HF}=7.5, J=2.6 Hz, 1H), 7.88-7.73 (m, 1H), 7.54 (ddd, ³J_{HF}=10.6, ⁴J_{HF}=9.2, J=9.2 Hz, 1H). ¹⁹F NMR (376 MHz, DMSO) δ −137.09 (d, ³J_{FF}=23.1 Hz), −142.53 (d, ³J_{FF}=23.1 Hz). Tr(MET-uPLC-AB2010)=0.68 min, m/z (ES⁺) [M+H]⁺ 294.0/296.0 (Cl³⁵/Cl³⁷), 94%.

### Step 2: Preparation of N-(3,4-difluorophenyl)-2-(4-ethylpiperazin-1-yl)pteridin-4-amine (Compound 16)

Prepared according to GP3, using DIPEA (45 μL, 0.18 mmol), 2-chloro-N-(3,4-difluorophenyl)pteridin-4-amine (Int-12, 94%, 45 mg, 0.15 mmol) and 1-ethylpiperazine (22 μL, 0.17 mmol) in anhydrous DMSO (1.4 mL) and the products were extracted into DCM from aqueous NaOH (pH 14). The product was separated by high pH prep. HPLC (ammonium hydroxide-water-acetonitrile) to afford Compound 16 after freeze drying.

¹H NMR (500 MHz, DMSO) δ 10.42 (s, 1H), 8.81 (d, J=2.0 Hz, 1H), 8.44 (d, J=2.0 Hz, 1H), 8.07 (ddd, ³J_{HF}=13.4, ⁴J_{HF}=7.5, J=2.6 Hz, 1H), 7.87-7.72 (m, 1H), 7.46 (ddd, ³J_{HF}=10.6, ⁴J_{HF}=9.2, J=9.2 Hz, 1H), 3.85 (br. s, 4H), 2.45 (app. t, J=5.1 Hz, 4H), 2.37 (q, J=7.2 Hz, 2H), 1.04 (t, J=7.1 Hz, 3H). ¹⁹F NMR (376 MHz, DMSO) δ −137.51 (d, ³J_{FF}=23.4 Hz), −144.36 (d, ³J_{FF}=23.3 Hz). Tr(MET-uHPLC-AB-107)=3.39 min, m/z (ES⁺) [M+H]⁺ 372.3, 100%.

### Example 7

### Preparation of N-benzyl-2-(4-methylpiperazin-1-yl)pteridin-4-amine (Compound 5)

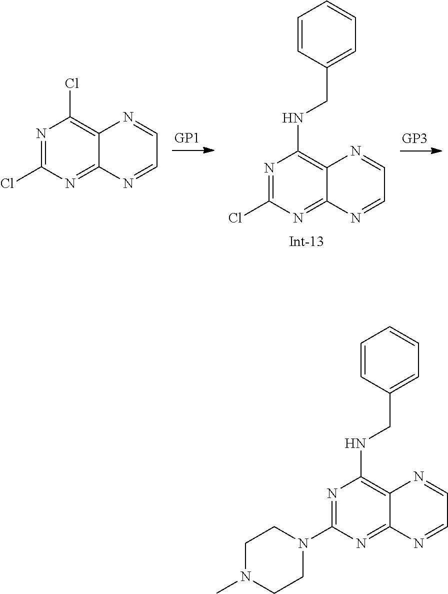 [SMILES: Clc1nc(Cl)c2nccnc2n1 ; Clc1nc(NCc2ccccc2)c2nccnc2n1 ; CN1CCN(c2nc(NCc3ccccc3)c3nccnc3n2)CC1]

### Step 1: Preparation of N-benzyl-2-chloro-pteridin-4-amine (Int-13)

Prepared according to GP1, using DIPEA (78 μL, 0.45 mmol), 2,4-dichloropteridine (50 mg, 0.25 mmol) and benzylamine (27 μL, 0.25 mmol) in IPA (0.75 mL) and the reaction mixture was concentrated in vacuo to afford the crude product. The product was separated by FCC (Biotage isolera, 25 g Sfar Duo column, eluent: 5 to 100% ethyl acetate in heptane) to afford Int-13 (50 mg, 68%).

¹H NMR (400 MHz, DMSO) δ 9.93 (s, 1H), 9.08 (d, J=2.0 Hz, 1H), 8.85 (d, J=2.0 Hz, 1H), 7.42-7.29 (m, 4H), 7.28-7.21 (m, 1H), 4.74 (s, 2H). Tr(MET-uPLC-AB2010)=0.64 min, m/z (ES⁺) [M+H]⁺ 272.1/274.0 (Cl³⁵/Cl³⁷), 100%.

### Step 2: Preparation of N-benzyl-2-(4-methylpiperazin-1-yl)pteridin-4-amine (Compound 5)

Prepared according to GP3, using DIPEA (58 μL, 0.33 mmol), N-benzyl-2-chloro-pteridin-4-amine (Int-13, 100%, 50 mg, 0.19 mmol) and 1-methylpiperazine (25 μL, 0.22 mmol) in anhydrous DMSO (1.7 mL) and the products were extracted into DCM from aqueous NaOH (pH 14). The product was separated by high pH prep. HPLC (ammonium hydroxide-water-acetonitrile) to afford Compound 5 after freeze drying.

¹H NMR (500 MHz, DMSO) δ 9.06 (t, J=6.3 Hz, 1H), 8.70 (d, J=2.1 Hz, 1H), 8.31 (d, J=2.1 Hz, 1H), 7.42-7.35 (m, 2H), 7.32-7.27 (m, 2H), 7.25-7.19 (m, 1H), 4.65 (d, J=6.3 Hz, 2H), 3.80 (app. t, J=5.1 Hz, 4H), 2.30 (app. t, J=5.1 Hz, 4H), 2.18 (s, 3H). Tr(MET-uHPLC-AB-107)=2.92 min, m/z (ES⁺) [M+H]⁺ 336.3, 100%.

### Example 8

### N-(3-chlorophenyl)-2-(4-methylpiperazin-1-yl)pteridin-4-amine (Compound 33)

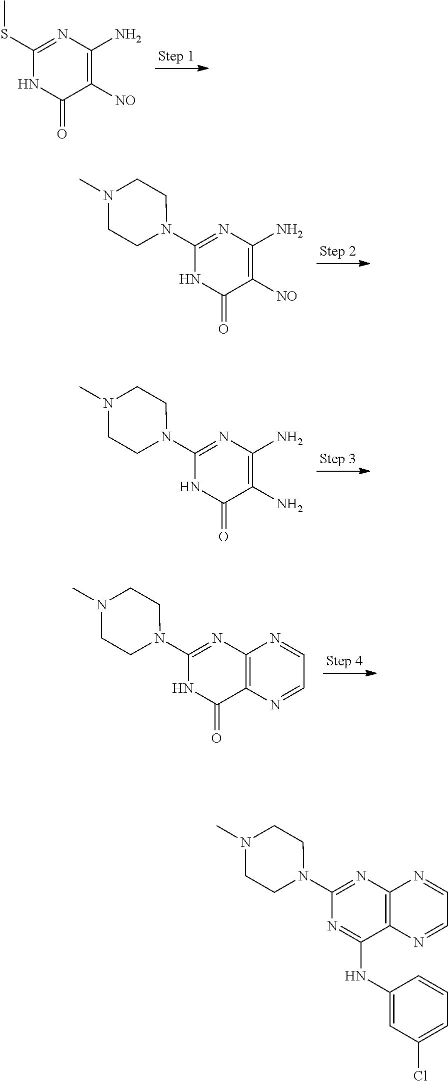

### Step 1: Preparation of 6-amino-2-(4-methylpiperazin-1-yl)-5-nitrosopyrimidin-4(3H)-one

4-Amino-2-methylsulfanyl-5-nitroso-1H-pyrimidin-6-one (500 mg, 2.69 mmol) was dissolved in water (15 mL) to give a violet solution. 1-Methylpiperazine (0.30 mL, 2.69 mmol) was added and the reaction was stirred at RT for 48 h. The water was removed in vacuo to give a residue. The residue was purified by SCX chromatography (20 g, eluting with 50% water/MeOH and releasing with 10% 7 M NH₃ in MeOH/MeOH). The solvent was removed in vacuo to give 6-amino-2-(4-methylpiperazin-1-yl)-5-nitrosopyrimidin-4(3H)-one (640 mg, 100%). LCMS (AQ7 generic basic run) RT=0.89 min, (ES+) 239 (M+H).

### Step 2: Preparation of 5,6-diamino-2-(4-methylpiperazin-1-yl)pyrimidin-4(3H)-one

4-Amino-2-(4-methylpiperazin-1-yl)-5-nitroso-1H-pyrimidin-6-one (640 mg, 2.69 mmol) was dissolved in water (7 mL) and 30% ammonium hydroxide solution (0.60 mL, 2.69 mmol) was added. The reaction was stirred for 20 minutes at RT. Sodium hydrosulfite (1169 mg, 6.72 mmol) was added and the reaction stirred for 16 h at 60° C. The solvent was removed in vacuo and azeotroped from toluene ×3 to give 5,6-diamino-2-(4-methylpiperazin-1-yl)pyrimidin-4(3H)-one. The crude material was used in the next step without further purification, assuming 100% yield. LCMS (AQ7 generic basic run) RT=0.55 min, (ES+) 255 (M+H).

### Step 3: Preparation of 2-(4-methylpiperazin-1-yl)pteridin-4(3H)-one

The crude 4,5-diamino-2-(4-methylpiperazin-1-yl)-1H-pyrimidin-6-one (602 mg, 2.69 mmol, assume 100% yield from previous step) was dissolved in methyl alcohol (15 mL) and 1 drop of 2 M HCl was added. Glyoxal solution (40% in water, 0.62 mL, 5.37 mmol) was added and the reaction was heated at reflux for 4 h. The reaction was cooled to RT and loaded directly onto an SCX cartridge (20 g), which was washed with water. The compound was released using 10% 7 N NH₃ in MeOH/MeOH. The basic fraction was concentrated in vacuo to give 2-(4-methylpiperazin-1-yl)pteridin-4(3H)-one (458 mg, 69%). LCMS (AQ7 generic basic run) RT=0.89 min, (ES+) 247 (M+H).

### Step 4: Preparation of N-(3-chlorophenyl)-2-(4-methylpiperazin-1-yl)pteridin-4-amine

8-Diazabicyclo[5.4.0]undec-7-ene (0.096 mL, 0.640 mmol) was added to a solution of 2-(4-methylpiperazin-1-yl)-3H-pteridin-4-one (105 mg, 0.426 mmol) and (benzotriazol-1-yloxy)tris(dimethylamino)phosphonium hexafluorophosphate (245 mg, 0.554 mmol) in anhydrous acetonitrile (3 mL). The solution was stirred at room temperature for 10 min and 3-chloroaniline (0.055 mL, 0.518 mmol) was added. The reaction was stirred at RT for 1 h and then at 60° C. for 16 h and cooled to RT. The reaction was cooled to RT and dry loaded onto silica before purification by silica chromatography (10 g, eluting with MeOH/DCM 0-10%) to give the target material (˜80 mg). The material was purified by preparative HPLC to give the title compound (15 mg, 12%). LCMS (ES+) 356 (M+H)+, RT (retention time) 2.93 min (Analytical method AcHSSC18); ¹H NMR (400 MHz, DMSO): δ 10.39 (s, 1H), 8.82 (d, J=2.0 Hz, 1H), 8.45 (d, J=2.0 Hz, 1H), 8.20 (dd, J=2.0, 2.0 Hz, 1H), 7.89 (dd, J=1.1, 8.2 Hz, 1H), 7.42 (dd, J=8.1, 8.1 Hz, 1H), 7.18-7.16 (m, 1H), 3.87 (dd, J=4.8, 4.8 Hz, 4H), 2.41 (dd, J=5.1, 5.1 Hz, 4H), 2.23 (s, 3H).

Further analogues were prepared using the same chemistry and commercially available anilines.

| Compound | | |
|---|---|---|
| No. | Structure | Analytical data |
| 30 | 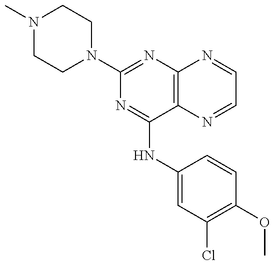 | Yield: 25 mg, 15% LCMS (ES+) 386 (M + H)+, RT 4.24 min (Analytical method BicarbBEHC18). ¹H NMR (400 MHz, DMSO): δ 10.26 (s, 1 H), 8.79 (d, J = 2.0 Hz, 1 H), 8.42 (d, J = 2.0 Hz, 1 H), 8.14 (d, J = 2.8 Hz, 1 H), 7.85 (dd, J = 2.5, 8.8 Hz, 1 H), 7.19 (d, J = 9.1 Hz, 1 H), 3.87 (s, 7 H), 2.40 (dd, J = 4.9, 4.9 Hz, 4 H), 2.22 (s, 3 H) |
| 34 | 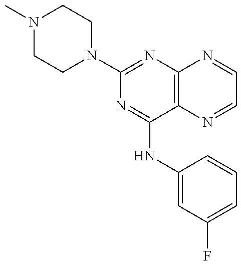 [SMILES: CN1CCN(c2nc(Nc3cccc(F)c3)c3nccnc3n2)CC1] | Yield: 7.3 mg, 6.2% LCMS (ES+) 340 (M + H)+, RT 2.71 min (Analytical method AcHSSC18). ¹H NMR (400 MHz, DMSO): δ 10.38 (s, 1 H), 8.83 (d, J = 2.3 Hz, 1 H), 8.45 (d, J = 2.0 Hz, 1 H), 7.93-7.88 (m, 1 H), 7.83-7.80 (m, 1 H), 7.46-7.39 (m, 1 H), 6.97- 6.92 (m, 1 H), 3.87 (dd, J = 4.8, 4.8 Hz, 4 H), 2.41 (dd, J = 4.9, 4.9 Hz, 4 H), 2.23 (s, 3 H). |

### Example 9

### N-(2,3-dimethylphenyl)-2-(4-methylpiperazin-1-yl)pteridin-4-amine (Compound 39)

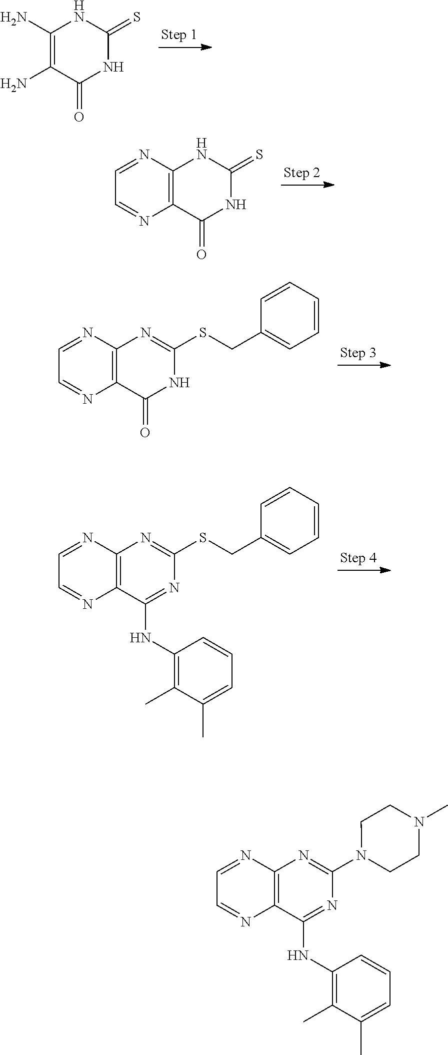 [SMILES: Nc1[nH]c(=S)[nH]c(=O)c1N ; O=c1[nH]c(=S)[nH]c2nccnc12 ; O=c1[nH]c(SCc2ccccc2)nc2nccnc12 ; Cc1cccc(Nc2nc(SCc3ccccc3)nc3nccnc23)c1C ; Cc1cccc(Nc2nc(N3CCN(C)CC3)nc3nccnc23)c1C]

### Step 1: Preparation of 2-thioxo-2,3-dihydropteridin-4(1H)-one

5,6-Diamino-2-thioxo-1H-pyrimidin-4-one (1000 mg, 6.32 mmol) was dissolved in methyl alcohol (30 mL) and glyoxal solution (40%, 3.6 mL, 31.6 mmol) was added. The reaction was heated at reflux for 3 h. The reaction was cooled to RT and a light brown solid formed. The solid was collected by filtering and the solid washed with cold MeOH to give 2-thioxo-2,3-dihydropteridin-4(1H)-one (750 mg, 66%). ¹H NMR (400 MHz, DMSO): δ 13.32-13.27 (m, 1H), 12.79 (s, 1H), 8.72 (d, J=2.3 Hz, 1H), 8.60 (d, J=2.3 Hz, 1H).

### Step 2: Preparation of 2-(benzylthio)pteridin-4(3H)-one

2-Thioxo-1H-pteridin-4-one (1040 mg, 5.77 mmol) was dissolved in tetrahydrofuran (20 mL) and 2 M sodium hydroxide (10 mL, 20.8 mmol) was added. Benzyl bromide (0.62 mL, 5.19 mmol) was added and the reaction stirred at RT for 18 h. The reaction was acidified with 2 M HCl to pH˜3. The aqueous layer was extracted with EtOAc ×3 and the organic layer dried by passing through phase separator paper. The organic layer was concentrated in vacuo to give a residue, which was purified by silica chromatography (10 g, eluting with EtOAc/cyclohexane) to give 2-(benzylthio)pteridin-4(3H)-one (900 mg, 58%). ¹H NMR (400 MHz, DMSO): δ 13.16 (s, 1H), 8.92 (d, J=2.0 Hz, 1H), 8.74 (d, J=2.1 Hz, 1H), 7.50 (d, J=7.0 Hz, 2H), 7.34 (dd, J=7.3, 7.3 Hz, 2H), 7.30-7.26 (m, 1H), 4.55 (s, 2H).

### Step 3: Preparation of 2-(benzylthio)-N-(2,3-dimethylphenyl)pteridin-4-amine

2-(Benzylthio)pteridin-4(3H)-one (70 mg, 0.259 mmol) was dissolved in acetonitrile (2 mL) and (benzotriazol-1-yloxy)tris(dimethylamino)phosphonium hexafluorophosphate (149 mg, 0.337 mmol) was added. 1,8-Diazabicyclo[5.4.0]undec-7-ene (0.058 mL, 0.388 mmol) was added dropwise and the reaction stirred at RT for 10 minutes. 2,3-Dimethylaniline (0.047 mL, 0.388 mmol) was added and the reaction stirred at 60° C. for 18 h. The reaction was cooled to RT and diluted with DCM and washed with water. The DCM layer was dried by passing through phase separator paper and the DCM removed in vacuo to give a residue. The residue was purified by silica chromatography (10 g, eluting with MeOH/DCM 0-10%) to give 2-(benzylthio)-N-(2,3-dimethylphenyl)pteridin-4-amine. LCMS (AQ7 generic basic run) RT=1.86 min, (ES+) 374 (M+H).

### Step 4: Preparation of N-(2,3-dimethylphenyl)-2-(4-methylpiperazin-1-yl)pteridin-4-amine

2-Benzylsulfanyl-N-(2,3-dimethylphenyl)pteridin-4-amine (81 mg, 0.217 mmol) was dissolved in dichloromethane (5 mL) and cooled to 0° C. using an ice-bath. 3-Chloroperbenzoic acid (77, 73 mg, 0.325 mmol) was added in one portion and the reaction stirred at 0° C. for 1 h. The reaction was analyzed by LCMS (AQ6 generic acidic run): the starting sulfide was converted to sulfoxide and was observed M+1=390 found at RT=1.57. The solvent was immediately removed in vacuo keeping the water bath at RT and the residue was immediately dissolved in acetonitrile (5 mL). 1-Methylpiperazine (0.072 mL, 0.651 mmol) was added and the reaction stirred at RT for 3 h. The solvent was removed in vacuo and the residue was purified by preparative HPLC. The sample was returned (60 mg). ¹H NMR indicated impurities >10% in the aromatic region. The sample was further purified by SFC to give the title compound (41 mg, 54%). LCMS (ES+) 350 (M+H)+, RT (retention time) 2.94 min (Analytical method AcHSSC18); ¹H NMR (400 MHz, DMSO): δ 9.81 (s, 1H), 8.78 (d, J=2.0 Hz, 1H), 8.41 (d, J=2.0 Hz, 1H), 7.42 (d, J=7.8 Hz, 1H), 7.13 (dd, J=7.7, 7.7 Hz, 1H), 7.08 (d, J=7.4 Hz, 1H), 3.71-3.70 (m, 4H), 2.35-2.25 (m, 7H), 2.15 (d, J=17.9 Hz, 6H).

Further analogues were prepared using the same chemistry and commercially available anilines.

| Compound | | |
|---|---|---|
| No. | Structure | Analytical data |
| 40 | 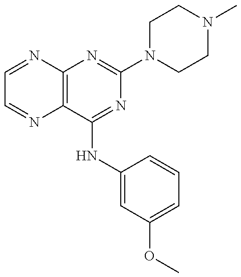 [SMILES: COc1cccc(Nc2nc(N3CCN(C)CC3)nc3nccnc23)c1] | Yield: 66 mg, 78% LCMS (ES+) 352 (M + H)+, RT 2.72 min (Analytical method AcHSSC18); ¹H NMR (400 MHz, DMSO): δ 10.13 (s, 1 H), 8.82 (d, J = 2.0 Hz, 1 H), 8.44 (d, J = 2.0 Hz, 1 H), 7.73 (dd, J = 2.2, 2.2 Hz, 1 H), 7.53 (d, J = 8.1 Hz, 1 H), 7.29 (dd, J = 8.1, 8.1 Hz, 1 H), 6.70 (dd, J = 2.4, 8.1 Hz, 1 H), 3.89 (dd, J = 4.8, 4.8 Hz, 4 H), 3.78 (s, 3 H), 2.41 (dd, J = 4.9, 4.9 Hz, 4 H), 2.23 (s, 3 H). |
| 41 | 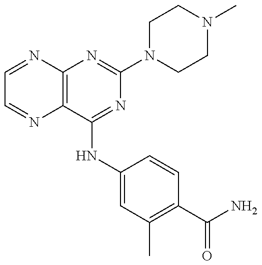 [SMILES: Cc1cc(Nc2nc(N3CCN(C)CC3)nc3nccnc23)ccc1C(N)=O] | Yield: 14 mg, 15% LCMS (ES+) 379 (M + H)+, RT 2.06 min (Analytical method AcHSSC18); ¹H NMR (400 MHz, DMSO): δ 10.23 (s, 1 H), 8.82 (d, J = 2.0 Hz, 1 H), 8.45 (d, J = 2.0 Hz, 1 H), 7.91 (s, 1 H), 7.78 (dd, J = 1.9, 8.4 Hz, 1 H), 7.67 (s, 1 H), 7.44 (d, J = 8.4 Hz, 1 H), 7.27 (s, 1 H), 3.89 (dd, J = 4.5, 4.5 Hz, 4 H), 2.42 (dd, J = 5.2, 5.2 Hz, 6 H), 2.24 (s, 3 H). |
| 42 | 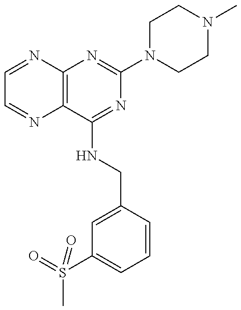 [SMILES: CN1CCN(c2nc(NCc3cccc(S(C)(=O)=O)c3)c3nccnc3n2)CC1] | Yield: 33 mg, 35% LCMS (ES+) 414 (M + H)+, RT 2.17 min (Analytical method AcHSSC18); ¹H NMR (400 MHz, DMSO): δ 9.22 (dd, J = 6.2, 6.2 Hz, 1 H), 8.72 (d, J = 2.1 Hz, 1 H), 8.33 (d, J = 2.0 Hz, 1 H), 8.00 (s, 1 H), 7.80 (d, J = 7.8 Hz, 1 H), 7.74 (d, J = 7.8 Hz, 1 H), 7.59 (dd, J = 7.8, 7.8 Hz, 1 H), 4.73 (d, J = 6.3 Hz, 2 H), 3.78 (dd, J = 4.5, 4.5 Hz, 4 H), 3.18 (s, 3 H), 2.33-2.27 (m, 4 H), 2.18 (s, 3 H). |
| 43 | 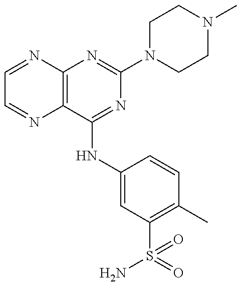 [SMILES: Cc1ccc(Nc2nc(N3CCN(C)CC3)nc3nccnc23)cc1S(N)(=O)=O] | Yield: 18 mg, 22% LCMS (ES+) 415 (M + H)+, RT 2.40 min (Analytical method AcHSSC18); ¹H NMR (400 MHz, DMSO): δ 10.39 (s, 1 H), 8.98 (d, J = 2.3 Hz, 1 H), 8.81 (d, J = 2.0 Hz, 1 H), 8.44 (d, J = 2.0 Hz, 1 H), 7.85 (dd, J = 2.3, 8.2 Hz, 1 H), 7.36 (d, J = 8.3 Hz, 1 H), 7.31 (s, 2 H), 3.90 (dd, J = 5.0, 5.0 Hz, 4 H), 2.57 (s, 3 H), 2.40 (dd, J = 4.8, 4.8 Hz, 4 H), 2.23 (s, 3 H). |
| 44 | 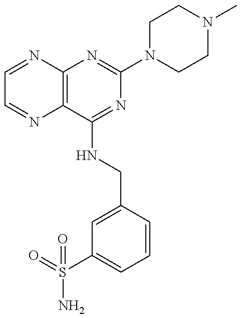 [SMILES: CN1CCN(c2nc(NCc3cccc(S(N)(=O)=O)c3)c3nccnc3n2)CC1] | Yield: 7.5 mg, 8.8% LCMS (ES+) 415 (M + H)+, RT 2.00 min (Analytical method AcHSSC18); ¹H NMR (400 MHz, DMSO): δ 9.20 (dd, J = 6.3, 6.3 Hz, 1 H), 8.71 (d, J = 2.0 Hz, 1 H), 8.33 (d, J = 2.1 Hz, 1 H), 7.88 (s, 1 H), 7.70 (d, J = 7.6 Hz, 1 H), 7.61 (d, J = 7.8 Hz, 1 H), 7.50 (dd, J = 7.7, 7.7 Hz, 1 H), 7.32 (s, 2 H), 4.71 (d, J = 6.1 Hz, 2 H), 3.78 (dd, J = 4.4, 4.4 Hz, 4 H), 2.33-2.30 (m, 4 H), 2.18 (s, 3 H). |
| 45 | 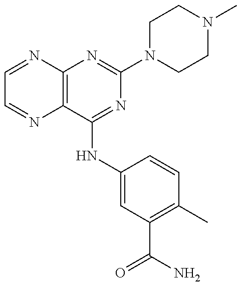 [SMILES: Cc1ccc(Nc2nc(N3CCN(C)CC3)nc3nccnc23)cc1C(N)=O] | Yield: 38 mg, 63% LCMS (ES+) 379 (M + H)+, RT 2.14 min (Analytical method AcHSSC18); ¹H NMR (400 MHz, DMSO): δ 10.23 (s, 1 H), 8.81 (d, J = 2.0 Hz, 1 H), 8.44 (d, J = 2.0 Hz, 1 H), 8.18 (d, J = 2.1 Hz, 1 H), 7.80 (dd, J = 2.3, 8.3 Hz, 1 H), 7.70 (s, 1 H), 7.37 (s, 1 H), 7.24 (d, J = 8.3 Hz, 1 H), 3.87 (dd, J = 4.6, 4.6 Hz, 4 H), 2.41-2.37 (m, 4 H), 2.35 (s, 3 H), 2.23 (s, 3 H). |
| 46 | 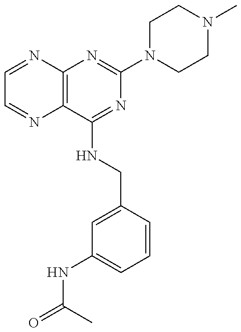 [SMILES: CC(=O)Nc1cccc(CNc2nc(N3CCN(C)CC3)nc3nccnc23)c1] | Yield: 20 mg, 19% LCMS (ES+) 393 (M + H)+, RT 2.14 min (Analytical method AcHSSC18); ¹H NMR (400 MHz, DMSO): δ 9.86 (s, 1 H), 9.06 (dd, J = 6.2, 6.2 Hz, 1 H), 8.71 (d, J = 2.1 Hz, 1 H), 8.32 (d, J = 2.0 Hz, 1 H), 7.58 (s, 1 H), 7.46 (dd, J = 1.4, 6.7 Hz, 1 H), 7.21 (dd, J = 7.8, 7.8 Hz, 1 H), 7.05 (d, J = 7.7 Hz, 1 H), 4.62 (d, J = 6.1 Hz, 2 H), 3.80 (dd, J = 4.6, 4.6 Hz, 4 H), 2.30 (dd, J = 5.1, 5.1 Hz, 4 H), 2.19 (s, 3 H), 2.00 (s, 3 H). |

### Example 10

### Compound 48 (Stereoisomer 1)

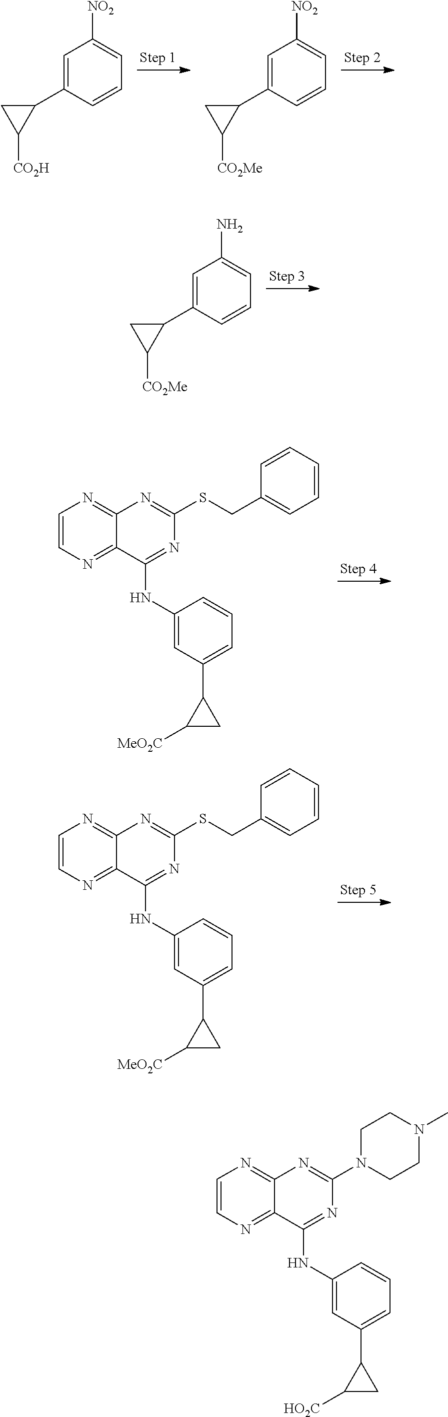 [SMILES: O=C(O)C1CC1c1cccc([N+](=O)[O-])c1 ; COC(=O)C1CC1c1cccc([N+](=O)[O-])c1 ; COC(=O)C1CC1c1cccc(N)c1 ; COC(=O)C1CC1c1cccc(Nc2nc(SCc3ccccc3)nc3nccnc23)c1 ; COC(=O)C1CC1c1cccc(Nc2nc(SCc3ccccc3)nc3nccnc23)c1 ; CN1CCN(c2nc(Nc3cccc(C4CC4C(=O)O)c3)c3nccnc3n2)CC1]

### Step 1: Preparation of methyl 2-(3-nitrophenyl)cyclopropane-1-carboxylate

A 1:1 mixture of cis and trans 2-(3-nitrophenyl)cyclopropanecarboxylic acid (250 mg, 1.21 mmol) was dissolved in N,N-dimethylformamide (3 mL) and cooled in an ice-bath. Potassium carbonate (500 mg, 3.62 mmol) was added followed by iodomethane (0.083 mL, 1.33 mmol, 1.10 eq). The reaction was allowed to warm to RT and stirred for 18 h. The reaction was diluted with EtOAc and washed with water. The organic layer was separated and was dried by passing through phase separator paper. The solvent was removed in vacuo to give a clear oil. The residue was purified by silica chromatography (10 g, eluting with EtOAc/cyclohexane 0-100%) to give a 1:1 mixture of cis and trans methyl 2-(3-nitrophenyl)cyclopropane-1-carboxylate (200 mg, 75%).

### Step 2: Preparation of methyl 2-(3-aminophenyl)cyclopropane-1-carboxylate

A 1:1 mixture of cis and trans methyl 2-(3-nitrophenyl)cyclopropane-1-carboxylate (250 mg, 1.13 mmol) was dissolved in ethanol (10 mL) and tin(II) chloride dihydrate (893 mg, 3.96 mmol) was added. The reaction was cooled to 0° C. in an ice-bath. Hydrochloric acid (0.5 mL, 37%) was added dropwise and the reaction allowed to warm to RT for 18 h. The reaction was quenched with 2 M sodium carbonate, and the aqueous layer was extracted with DCM. The layers were separated using a phase separator and the DCM removed in vacuo to give a residue. The residue was purified by silica chromatography (10 g, eluting with EtOAc/cyclohexane 0-100%) to give a 1:1 cis and trans mixture of methyl 2-(3-aminophenyl)cyclopropane-1-carboxylate (200 mg, 93%).

### Step 3: Preparation of methyl 2-[3-[(2-benzylsulfanylpteridin-4-yl)amino]phenyl]cyclopropanecarboxylate

2-Benzylsulfanyl-3H-pteridin-4-one (188 mg, 0.695 mmol) was dissolved in acetonitrile (2 mL) and (benzotriazol-1-yloxy)tris(dimethylamino)phosphonium hexafluorophosphate (400 mg, 0.904 mmol) was added. 1,8-Diazabicyclo[5.4.0]undec-7-ene (0.16 mL, 1.04 mmol) was added dropwise and the reaction stirred at RT for 10 minutes. A 1:1 mixture of cis and trans methyl 2-(3-aminophenyl)cyclopropane-1-carboxylate (200 mg, 1.04 mmol) was added and the reaction stirred at 60° C. for 18 h. The reaction was diluted with DCM and washed with water. The DCM layer was dried by passing through phase separator paper and the DCM removed in vacuo to give a residue. The residue was purified by silica chromatography (10 g, eluting with MeOH/DCM 0-10%) to give cis and trans methyl 2-[3-[(2-benzylsulfanylpteridin-4-yl)amino]phenyl]cyclopropanecarboxylate (250 mg, 81%). LCMS (acidic): M+1=444 found at 1.82 and 1.85 mins corresponding to cis and trans isomers.

### Step 4: Preparation of methyl 2-[3-[[2-(4-methylpiperazin-1-yl)pteridin-4-yl]amino]phenyl]cyclopropanecarboxylate

A 1:1 mixture of cis and trans methyl 2-[3-[(2-benzylsulfanylpteridin-4-yl)amino]phenyl]cyclopropanecarboxylate (250 mg, 0.564 mmol) was dissolved in tetrahydrofuran (5 mL) and cooled to 0° C. using an ice-bath. 3-Chloroperbenzoic acid (77%, 164 mg, 0.733 mmol) was added in one portion and the reaction stirred at 0° C. for 1 h. 1-Methylpiperazine (0.19 mL, 1.69 mmol) was added, and the reaction stirred at RT for 4 h. The solvent was removed in vacuo and the residue purified by SFC to give a 1:1 mixture of cis and trans methyl 2-[3-[[2-(4-methylpiperazin-1-yl)pteridin-4-yl]amino]phenyl]cyclopropanecarboxylate (91 mg, 38%). LCMS (basic): M+1=420 found at RT=1.61 and 1.67 mins.

### Step 5: Preparation of Compound 48 (Stereoisomer 1)

A 1:1 mixture of cis and trans methyl 2-[3-[[2-(4-methylpiperazin-1-yl)pteridin-4-yl]amino]phenyl]cyclopropanecarboxylate (91 mg, 0.217 mmol) was dissolved in methyl alcohol (0.5 mL) and water (0.5 mL) and lithium hydroxide monohydrate (10 mg, 0.239 mmol) was added at RT. The reaction was stirred for 18 h at RT. The crude reaction mixture was purified by preparative HPLC to give the title compound as the “First Eluting Stereoisomer” (Stereoisomer 1) (14 mg, 16%). Based on NOESY NMR data of Example 11, it is contemplated that the majority product is the cis isomer. LCMS (ES+) 406 (M+H)+, RT (retention time) 2.59 min (Analytical method AcHSSC18); ¹H NMR (400 MHz, DMSO): δ 10.10 (s, 1H), 8.80 (d, J=1.9 Hz, 1H), 8.43 (d, J=1.9 Hz, 1H), 8.08 (s, 1H), 7.65 (d, J=8.1 Hz, 1H), 7.25 (dd, J=7.9, 7.9 Hz, 1H), 6.99 (d, J=7.6 Hz, 1H), 3.89 (s, 4H), 2.67-2.54 (m, 1H), 2.41 (dd, J=4.8, 4.8 Hz, 4H), 2.23 (s, 3H), 2.03 (dd, J=7.7, 14.8 Hz, 1H), 1.49 (dd, J=5.3, 12.2 Hz, 1H), 1.33-1.26 (m, 1H).

### Example 11

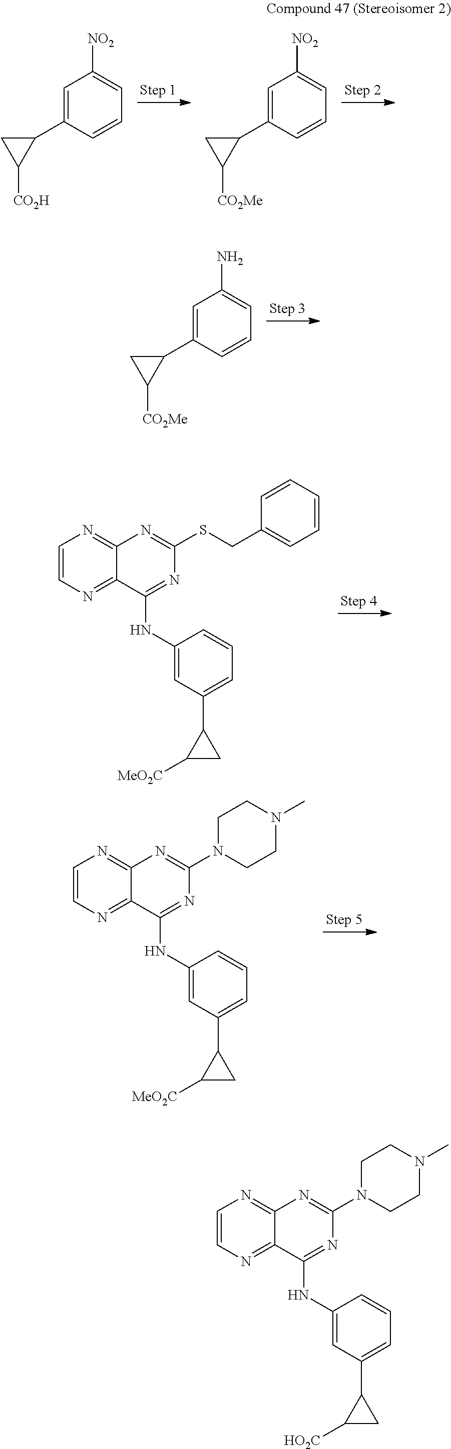 [SMILES: O=C(O)C1CC1c1cccc([N+](=O)[O-])c1 ; COC(=O)C1CC1c1cccc([N+](=O)[O-])c1 ; COC(=O)C1CC1c1cccc(N)c1 ; COC(=O)C1CC1c1cccc(Nc2nc(SCc3ccccc3)nc3nccnc23)c1 ; COC(=O)C1CC1c1cccc(Nc2nc(N3CCN(C)CC3)nc3nccnc23)c1 ; CN1CCN(c2nc(Nc3cccc(C4CC4C(=O)O)c3)c3nccnc3n2)CC1]

### Step 1

2-(3-Nitrophenyl)cyclopropanecarboxylic acid (250 mg, 1.21 mmol) was dissolved in N,N-dimethylformamide (3 mL) and cooled in an ice-bath. Potassium carbonate (500 mg, 3.62 mmol) was added followed by iodomethane (0.083 mL, 1.33 mmol). The reaction was allowed to warm to RT and stirred for 16 h. The reaction was diluted with EtOAc and washed with water. The organic layer was separated and was dried by passing through phase separator paper. The solvent was removed in vacuo to give methyl 2-(3-nitrophenyl)cyclopropanecarboxylate (250 mg, 94%) as a 1:1 mixture of cis and trans isomers. The diastereomers were separated by SFC (Conditions: LUX Cellulose-iC5 10×250 mm, 5 μm, 20/80 IPA (0.1% NH₄OH)/CO₂, 15 mL/min, 120 bar, 40° C., DAD 260 nm).

Isomer 1 (59 mg, 22%). RT (retention time) 1.43 min. ¹H NMR (400 MHz, CDCl₃): δ 8.09-8.05 (m, 1H), 7.94 (s, 1H), 7.47-7.45 (m, 2H), 3.74 (s, 3H), 2.63 (ddd, J=4.2, 6.4, 9.2 Hz, 1H), 2.01-1.96 (m, 1H), 1.73-1.68 (m, 1H), 1.40 (ddd, J=4.9, 6.5, 8.6 Hz, 1H). Based on NOESY spectra, it is contemplated that the majority product is the trans isomer. This product was taken forward into the next step.

Isomer 2 (38 mg, 14%). RT (retention time) 1.78 min. ¹H NMR (400 MHz, CDCl₃): δ 8.15 (s, 1H), 8.09-8.06 (m, 1H), 7.60 (d, J=7.7 Hz, 1H), 7.44 (dd, J=8.0, 8.0 Hz, 1H), 3.48 (s, 3H), 2.64 (q, J=8.4 Hz, 1H), 2.23-2.17 (m, 1H), 1.79-1.74 (m, 1H), 1.50-1.44 (m, 1H). Based on NOESY spectra, it is contemplated that the majority product is the cis isomer.

### Step 2

Hydrochloric Acid (0.1 mL, 37% solution) was added to a solution of Isomer 1 of Step 1 (59 mg, 0.267 mmol) and tin(II) chloride dihydrate (211 mg, 0.934 mmol) in ethanol (3 mL) at 0° C. The reaction was stirred at RT for 16 h. The reaction was quenched by addition of 2 M sodium carbonate solution and extracted with DCM. The organic layer was dried by passing through a phase separator paper and the solvent removed in vacuo to give a residue. The residue was purified by silica chromatography (10 g, eluting with EtOAc/cyclohexane 0-100%) to give the desired product (53 mg, >90%).

LCMS (AQ6) M+1=192 found at 1.15 mins.

### Step 3

2-Benzylsulfanyl-3H-pteridin-4-one (50 mg, 0.185 mmol) was dissolved in acetonitrile (2 mL) and (benzotriazol-1-yloxy)tris(dimethylamino)phosphonium hexafluorophosphate (106 mg, 0.240 mmol) was added. 1,8-Diazabicyclo[5.4.0]undec-7-ene (0.041 mL, 0.277 mmol) was added dropwise and the reaction stirred at RT for 10 minutes. The product of Step 2 (53 mg, 0.277 mmol) was added and the reaction stirred at 60° C. for 16 h. The reaction was diluted with DCM and washed with water. The DCM layer was dried by passing through phase separator paper and the DCM removed in vacuo to give a residue. The residue was purified by silica chromatography (10 g, eluting with MeOH/DCM 0-10%) to give the desired product (54 mg, 66%). LCMS (acidic): M+1=444 found at RT=1.46 mins.

### Step 4

The desired product of Step 3 (54 mg, 0.122 mmol) was dissolved in tetrahydrofuran (5 mL) and cooled to 0° C. using an ice-bath. 3-Chloroperbenzoic acid (77%, 35 mg, 0.158 mmol) was added in one portion and the reaction stirred at 0° C. for 1 h. 1-Methylpiperazine (0.041 mL, 0.365 mmol) was added and the reaction stirred at RT for 3 h. The solvent was removed in vacuo and the residue purified by silica chromatography (10 g, eluting with EtOAc/cyclohexane 0-100%) to give the desired product (46 mg, 0.110 mmol, 90%).

### Step 5: Preparation of Compound 47 (Stereoisomer 2)

The desired product of Step 4 (46 mg, 0.110 mmol) was dissolved in water (0.5 mL) and methyl alcohol (0.5 mL) and lithium hydroxide monohydrate (4.8 mg, 0.115 mmol) was added. The reaction was stirred at RT for 16 h. The crude reaction mixture was purified by preparative HPLC to give the title compound (20 mg, 45%) as the “Second Eluting Stereoisomer” (Stereoisomer 2). Based on NOESY spectra of the intermediate as described herein, it is contemplated that the majority product is the trans isomer. LCMS (ES+) 406 (M+H)+, RT (retention time) 2.65 min (Analytical method AcHSSC18); ¹H NMR (400 MHz, DMSO): δ 10.13 (s, 1H), 8.81 (d, J=2.0 Hz, 1H), 8.43 (d, J=2.0 Hz, 1H), 7.82 (s, 1H), 7.71 (d, J=9.4 Hz, 1H), 7.30 (t, J=7.7 Hz, 1H), 7.00 (d, J=7.7 Hz, 1H), 3.90-3.85 (m, 4H), 2.45-2.40 (m, 5H), 1.83-1.77 (m, 1H), 1.49-1.42 (m, 1H), 1.38-1.31 (m, 1H).

### Example 12

### N-(3,4-dimethylphenyl)-6-methyl-2-(4-methylpiperazin-1-yl)pteridin-4-amine (Compound 38)

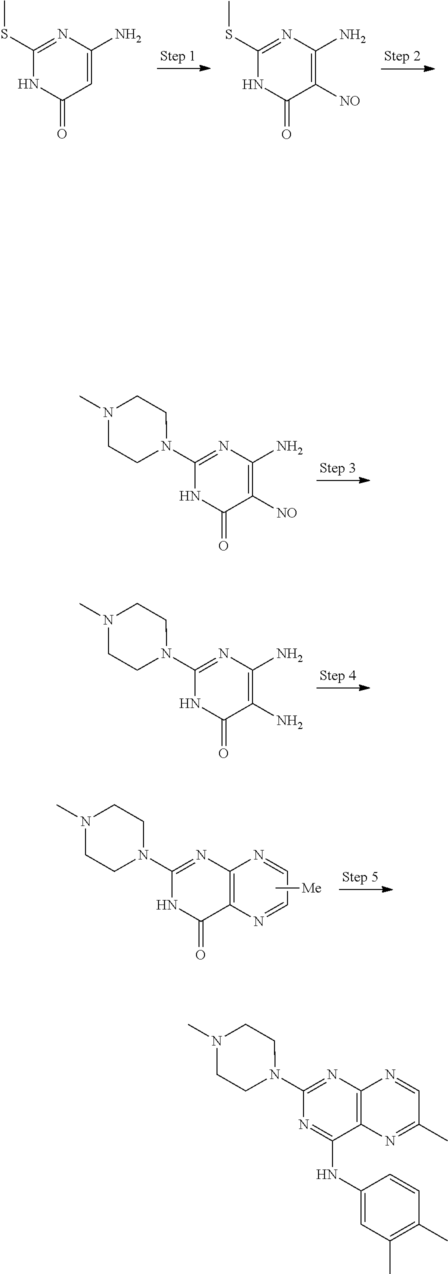

### Step 1: Preparation of 6-amino-2-(methylthio)-5-nitrosopyrimidin-4(3H)-one

4-Amino-2-methylsulfanyl-1H-pyrimidin-6-one (2000 mg, 12.7 mmol) was dissolved in dimethyl sulfoxide (50 mL) and isopentyl nitrite (1.9 mL, 14.0 mmol) was added. The reaction was stirred for 16 h at RT. The reaction was diluted with water to form a purple solid, which was collected by filtration. The purple solid was washed with water and dried in a vacuum oven overnight to give 6-amino-2-(methylthio)-5-nitrosopyrimidin-4(3H)-one (2.1 g, 89%). ¹H NMR (400 MHz, DMSO): δ 12.73 (s, 1H), 11.27-11.24 (m, 1H), 9.08-9.07 (m, 1H), 2.55 (s, 3H).

### Step 2: Preparation of 6-amino-2-(4-methylpiperazin-1-yl)-5-nitrosopyrimidin-4(3H)-one

6-Amino-2-(methylthio)-5-nitrosopyrimidin-4(3H)-one (500 mg, 2.69 mmol) was dissolved in water (15 mL) to give a violet solution. 1-Methylpiperazine (0.30 mL, 2.69 mmol) was added, and the reaction was stirred at RT for 48 h. The water was removed in vacuo to give a residue. The residue was purified by SCX chromatography (20 g, eluting with 50% water/MeOH and releasing with 10% 7 M NH₃ in MeOH/MeOH). The solvent was removed in vacuo to give 6-amino-2-(4-methylpiperazin-1-yl)-5-nitrosopyrimidin-4(3H)-one (640 mg, 100%). LCMS (basic) RT=0.89 min, (ES+) 239 (M+H).

### Step 3: Preparation of 5,6-diamino-2-(4-methylpiperazin-1-yl)pyrimidin-4(3H)-one

6-amino-2-(4-methylpiperazin-1-yl)-5-nitrosopyrimidin-4(3H)-one (640 mg, 2.69 mmol) was dissolved in water (7 mL) and 30% ammonium hydroxide solution (0.60 mL, 2.69 mmol) was added. The reaction was stirred for 20 minutes at RT. Sodium hydrosulfite (1169 mg, 6.72 mmol) was added, and the reaction stirred for 16 h at 60° C. The solvent was removed in vacuo and azeotroped from toluene ×3 to give 5,6-diamino-2-(4-methylpiperazin-1-yl)pyrimidin-4(3H)-one. The crude material was used in the next step without further purification, assuming 100% yield. LCMS (basic) RT=0.55 min, (ES+) 255 (M+H).

### Step 4: Preparation of 6-Methyl-2-(4-methylpiperazin-1-yl)-3H-pteridin-4-one

5,6-diamino-2-(4-methylpiperazin-1-yl)pyrimidin-4(3H)-one (602 mg, 2.69 mmol) was dissolved in methyl alcohol (20 mL) and 1 drop of 2M HCl was added followed by 2-oxopropanal oxime (702 mg, 8.06 mmol). The reaction was heated at reflux for 4 h. The reaction was cooled to RT. The reaction was loaded onto an SCX cartridge, which was washed with water. The compound was released using 10% 7 N NH₃ in MeOH/MeOH. The solvent was removed in vacuo to give 6-methyl-2-(4-methylpiperazin-1-yl)-3H-pteridin-4-one and 7-methyl-2-(4-methylpiperazin-1-yl)-3H-pteridin-4-one (700 mg, 2.69 mmol, 100%). LCMS (basic) M+1=261 found at RT=0.92 mins.

### Step 5: Preparation of N-(3,4-dimethylphenyl)-6-methyl-2-(4-methylpiperazin-1-yl)pteridin-4-amine

A mixture of 6-methyl-2-(4-methylpiperazin-1-yl)-3H-pteridin-4-one and 7-methyl-2-(4-methylpiperazin-1-yl)-3H-pteridin-4-one (3:2, 1.20 g, 4.61 mmol) was dissolved in N,N-dimethylformamide (20 mL), and (benzotriazol-1-yloxy)tris(dimethylamino)phosphonium hexafluorophosphate (2651 mg, 5.99 mmol) was added. 1,8-diazabicyclo[5.4.0]undec-7-ene (1.0 mL, 6.92 mmol) was added dropwise and the reaction stirred at RT for 10 minutes. 3,4-Dimethylaniline (838 mg, 6.92 mmol) was added, and the reaction stirred at 60° C. for 16 h. LCMS (acidic) showed target material as a minor component (two isomers <10%: M+1=364 found at RT=1.82 and 1.86 mins). The DMF was removed in vacuo to give a dark oil residue, which was dissolved in DCM and washed with water. The layers were separated, and the DCM layer dried by passing through phase separator paper. The DCM was removed in vacuo to give a residue, which was purified by silica chromatography (25 g, eluting with MeOH/DCM 0-10%) to give the target material as a yellow oil. LCMS analysis showed further purification was required. Purification by SFC yielded the target material (57 mg) as two regio-isomers. The material was further purified by chiral SFC to separate the two regio-isomers to give the title compound (28 mg, 1.7%). The regio-chemistry of the methyl pteridine was confirmed by HMBC. LCMS (ES+) 364 (M+H)+, RT (retention time) 3.23 min (Analytical method AcHSSC18); ¹H NMR (400 MHz, DMSO): δ 9.68 (s, 1H), 8.71 (s, 1H), 7.75 (s, 1H), 7.62 (dd, J=2.4, 8.3 Hz, 1H), 7.14 (d, J=8.2 Hz, 1H), 3.86-3.80 (m, 4H), 2.42-2.35 (m, 4H), 2.24-2.20 (m, 9H).

### Example 13

### N-(3,4-dimethylphenyl)-2-(4-methyl-1,4-diazepan-1-yl)pteridin-4-amine (Compound 23)

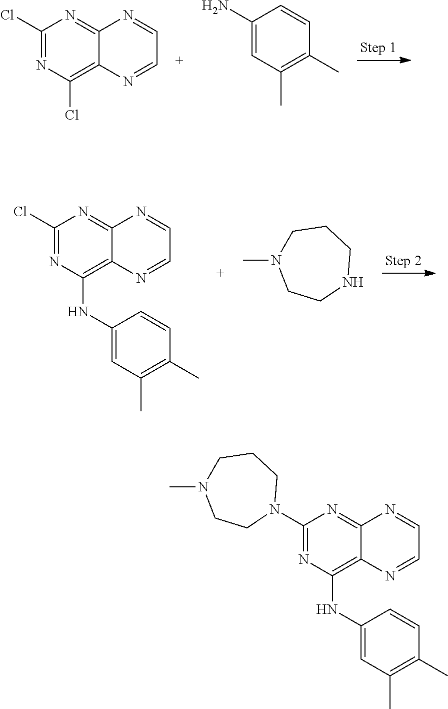

### Step 1: Preparation of 2-chloro-N-(3,4-dimethylphenyl)pteridin-4-amine

A solution of 3,4-dimethylaniline (121 mg, 0.999 mmol, 1.00 eq) and N,N-diisopropylethylamine (179 uL, 1.03 mmol, 1.03 eq) in dichloromethane (3.00 mL) was added to a suspension of 2,4-dichloropteridine (200 mg, 0.995 mmol, 1.00 eq) in dichloromethane (3.00 mL) cooled in ice over 5 minutes. The cooling was removed, and the reaction stirred at ambient for 1 hour. Dichloromethane (15 mL) and water (5 mL) were added (pH˜5) separated and the aqueous extracted with dichloromethane (10 mL). the organics were dried (MgSO₄) and evaporated to give 367 mg of orange solid. The crude was purified by flash chromatography elution gradient 10-100% EtOAc in cyclohexane to give 2-chloro-N-(3,4-dimethylphenyl)pteridin-4-amine (226 mg) LCMS (AQ6 generic acidic run) RT (retention time) 1.63 min, (ES+) 286.0 (M+H). ¹H NMR (400 MHz, CDCl3) δ 9.06 (d, J=1.9 Hz, 1H), 9.03 (s, 1H), 8.71 (d, J=2.0 Hz, 1H), 7.69 (dd, J=2.3, 8.2 Hz, 1H), 7.57 (d, J=2.1 Hz, 1H), 7.21 (d, J=8.2 Hz, 1H), 2.33 (s, 3H), 2.29 (s, 3H).

### Step 2: Preparation of N-(3,4-dimethylphenyl)-2-(4-methyl-1,4-diazepan-1-yl)pteridin-4-amine

A solution of 1-methylhomopiperazine (92 uL, 0.740 mmol, 5.03 eq) in dry dimethyl sulfoxide (1.00 mL) was added to 2-chloro-N-(3,4-dimethylphenyl)pteridin-4-amine (42 mg, 0.147 mmol, 1.00 eq) and the reaction heated to 60° C. for 1 hour. The reaction was cooled and crude material was purified by prep HPLC to give N-(3,4-dimethylphenyl)-2-(4-methyl-1,4-diazepan-1-yl)pteridin-4-amine (29 mg). LCMS (ES+) 364.3 (M+H)+, RT (retention time) 2.99 min (Analytical method AcHSSC18). ¹H NMR (400 MHz, DMSO) δ 9.95 (s, 1H), 8.77 (d, J=2.0 Hz, 1H), 8.39 (d, J=2.0 Hz, 1H), 7.91 (d, J=18.5 Hz, 1H), 7.65 (dd, J=8.7, 12.3 Hz, 1H), 7.13 (d, J=8.2 Hz, 1H), 3.93 (ddd, J=4.9, 4.9, 4.9 Hz, 2H), 3.87 (t, J=6.4 Hz, 2H), 2.72-2.68 (m, 2H), 2.29 (s, 3H), 2.25 (s, 3H), 2.22 (s, 3H), 1.93 (dd, J=5.8, 11.0 Hz, 2H). Additional homopiperazine signal under DMSO peak

Further analogues were prepared using the same chemistry and commercially available amines or phenols.

| Compound | | |
|---|---|---|
| No. | Structure | Analytical data |
| 24 | 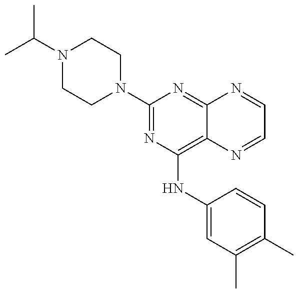 [SMILES: Cc1ccc(Nc2nc(N3CCN(C(C)C)CC3)nc3nccnc23)cc1C] | LCMS (ES+) 378.3 (M + H)+, RT 5.28 min (Analytical method BicarbBEHC18) ¹H NMR (400 MHz, DMSO) δ 9.99 (s, 1H), 8.78 (d, J = 2.0 Hz, 1H), 8.41 (d, J = 2.0 Hz, 1H), 7.78 (d, J = 1.8 Hz, 1H), 7.64 (dd, J = 2.1, 8.1 Hz, 1H), 7.17-7.13 (m, 1H), 3.87-3.83 (m, 4H), 2.75-2.69 (m, 1H), 2.25 (s, 3H), 2.22 (s, 3H), 1.01 (d, J = 6.5 Hz, 6H). 4 piperazine protons under DMSO peak |
| 31 | 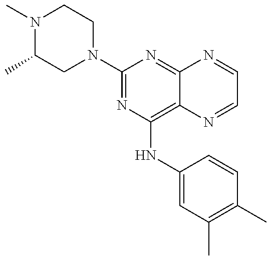 | LCMS (ES+) 364.4 (M + H)+, RT 4.92 min (Analytical method BicarbBEHC18) ¹H NMR (400 MHz, DMSO) δ 10.06 (s, 1H), 8.83 (d, J = 2.0 Hz, 1H), 8.46 (d, J = 2.0 Hz, 1H), 7.90 (s, 1H), 7.64 (d, J = 7.8 Hz, 1H), 7.20 (d, J = 8.1 Hz, 1H), 4.64- 4.55 (m, 2H), 3.27-3.20 (m, 1H), 2.92-2.84 (m, 2H), 2.30 (s, 3H), 2.27 (s, 6H), 2.18 (ddd, J = 11.4, 11.4, 3.4 Hz, 1H), 2.14-2.06 (m, 1H), 1.13 (d, J = 6.3 Hz, 3H). |
| 25 | 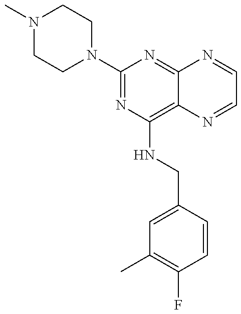 [SMILES: Cc1cc(CNc2nc(N3CCN(C)CC3)nc3nccnc23)ccc1F] | LCMS (ES+)368.4 (M + H)+, RT 4.35 min (Analytical method BicarbBEHC18) ¹H NMR (400 MHz, DMSO) δ 9.05 (dd, J = 6.1, 6.1 Hz, 1H), 8.71 (d, J = 2.0 Hz, 1H), 8.31 (d, J = 2.1 Hz, 1H), 7.31 (d, J = 7.8 Hz, 1H), 7.27-7.21 (m, 1H), 7.08-7.03 (m, 1H), 4.59 (d, J = 6.1 Hz, 2H), 3.83 (dd, J = 4.8, 4.8 Hz, 4H), 2.33 (dd, J = 4.8, 4.8 Hz, 4H), 2.20 (s, 3H), 2.19 (s, 3H). |
| 26 | 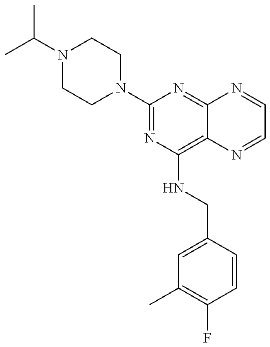 [SMILES: Cc1cc(CNc2nc(N3CCN(C(C)C)CC3)nc3nccnc23)ccc1F] | LCMS (ES+) 396.5 (M + H)+, RT 4.85 min (Analytical method BicarbBEHC18) ¹H NMR (400 MHz, DMSO) δ 9.04 (dd, J = 6.2, 6.2 Hz, 1H), 8.70 (d, J = 2.1 Hz, 1H), 8.31 (d, J = 2.1 Hz, 1H), 7.31 (d, J = 7.5 Hz, 1H), 7.26-7.21 (m, 1H), 7.09-7.03 (m, 1H), 4.59 (d, J = 6.3 Hz, 2H), 3.82-3.82 (m, 4H), 2.73-2.67 (m, 1H), 2.48-2.43 (m, 4H), 2.20 (d, J = 1.6 Hz, 3H), 0.99 (d, J = 6.5 Hz, 6H). |
| 27 | 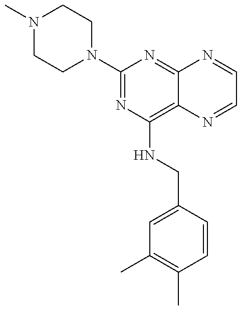 [SMILES: Cc1ccc(CNc2nc(N3CCN(C)CC3)nc3nccnc23)cc1C] | LCMS (ES+) 364.4 (M + H)+, RT 4.58 min (Analytical method BicarbBEHC18) ¹H NMR (400 MHz, DMSO) δ 8.99 (dd, J = 6.3, 6.3 Hz, 1H), 8.70 (d, J = 2.1 Hz, 1H), 8.30 (d, J = 2.0 Hz, 1H), 7.18 (s, 1H), 7.11 (d, J = 7.8 Hz, 1H), 7.05 (d, J = 7.8 Hz, 1H), 4.57 (d, J = 6.3 Hz, 2H), 3.83 (dd, J = 4.6, 4.6 Hz, 4H), 2.33 (dd, J = 4.8, 4.8 Hz, 4H), 2.20 (s, 3H), 2.18 (s, 3H), 2.17 (s, 3H). |
| 28 | 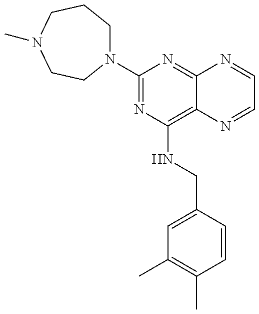 [SMILES: Cc1ccc(CNc2nc(N3CCCN(C)CC3)nc3nccnc23)cc1C] | LCMS (ES+) 378.3 (M + H)+, RT 2.7 min (Analytical method AcHSSC18) ¹H NMR (400 MHz, DMSO) δ 8.63 (d, J = 2.1 Hz, 1H), 8.10 (t, J = 2.4 Hz, 1H), 7.16 (s, 1H), 7.13-7.08 (m, 3H), 4.70 (dd, J = 5.9, 8.0 Hz, 2H), 4.11 (t, J = 4.8 Hz, 1H), 4.05-3.99 (m, 2H), 3.95 (t, J = 6.6 Hz, 1H), 2.76 (dd, J = 4.8, 4.8 Hz, 1H), 2.65 (t, J = 4.6 Hz, 1H), 2.58 (dd, J = 4.6, 10.0 Hz, 2H), 2.37 (d, J = 9.3 Hz, 3H), 2.26 (s, 6H), 2.08-1.95 (m, 2H). |
| 29 | 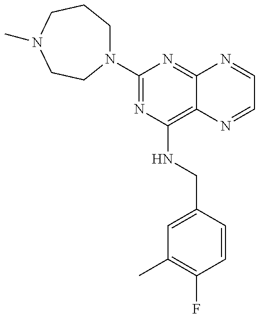 [SMILES: Cc1cc(CNc2nc(N3CCCN(C)CC3)nc3nccnc23)ccc1F] | LCMS (ES+) 382.3 (M + H)+, RT 2.63 min (Analytical method AcHSSC18) ¹H NMR (400 MHz, CDCl3) δ 8.67 (d, J = 2.0 Hz, 1H), 8.16 (s, 1H), 7.23-7.15 (m, 3H), 6.97 (dd, J = 8.9, 8.9 Hz, 1H), 4.69 (dd, J = 6.1, 9.2 Hz, 2H), 4.20-4.14 (m, 1H), 4.06-4.03 (m, 2H), 3.96 (t, J = 6.5 Hz, 1H), 2.98 (s, 1H), 2.79 (s, 3H), 2.48 (d, J = 18.2 Hz, 3H), 2.26 (s, 3H), 2.20-2.10 (m, 2H) |
| 35 | 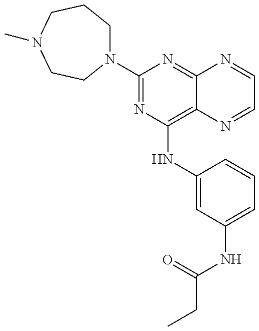 [SMILES: CCC(=O)Nc1cccc(Nc2nc(N3CCCN(C)CC3)nc3nccnc23)c1] | LCMS (ES+) 407.3 (M + H)+, RT 2.37 min (Analytical method AcHSSC18) ¹H NMR (400 MHz, CDCl3) δ 8.90 (s, 1H), 8.71 (d, J = 2.0 Hz, 1H), 8.36 (s, 1H), 8.23-8.22 (m, 1H), 7.75- 7.64 (m, 1H), 7.33 (dd, J = 8.0, 8.0 Hz, 1H), 7.16 (s, 1H), 6.99 (d, J = 8.0 Hz, 1H), 4.17-4.08 (m, 2H), 4.04 (tt, J = 6.6, 6.6 Hz, 2H), 2.81-2.76 (m, 2H), 2.64-2.58 (m, 2H), 2.42 (q, J = 6.9 Hz, 2H), 2.39 (s, 3H), 2.11-2.03 (m, 2H), 1.27 (dd, J = 7.5, 7.5 Hz, 3H). |
| 37 | 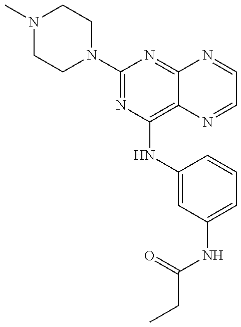 [SMILES: CCC(=O)Nc1cccc(Nc2nc(N3CCN(C)CC3)nc3nccnc23)c1] | LCMS (ES+) 393.2 (M + H)+, RT 2.49 min (Analytical method AcHSSC18 ¹H NMR (400 MHz, DMSO) δ 10.05 (s, 1H), 9.87 (s, 1H), 8.80 (d, J = 2.0 Hz, 1H), 8.62 (s, 1H), 8.43 (d, J = 2.0 Hz, 1H), 7.45 (d, J = 8.8 Hz, 1H), 7.28 (dd, J = 8.0, 8.0 Hz, 1H), 7.11 (d, J = 8.5 Hz, 1H), 3.89 (dd, J = 5.0, 5.0 Hz, 4H), 2.40 (t, J = 4.9 Hz, 3H), 2.34 (dd, J = 7.6, 15.0 Hz, 2H), 2.23 (s, 3H), 1.10 (dd, J = 7.5, 7.5 Hz, 3H). |
| 32 | 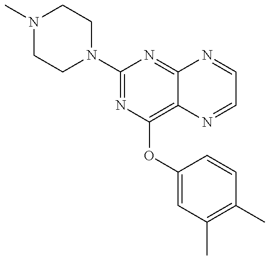 | LCMS (ES+) 351.5 (M + H)+, RT 4.34 min (Analytical method BicarbBEHC18) ¹H NMR (400 MHz, DMSO) δ 8.89 (d, J = 2.0 Hz, 1H), 8.57 (d, J = 2.0 Hz, 1H), 7.24 (d, J = 8.1 Hz, 1H), 7.14 (d, J = 2.5 Hz, 1H), 7.07 (dd, J = 2.5, 8.1 Hz, 1H), 3.89-3.52 (m, 4H), 2.35-2.27 (m, 4H), 2.26 (s, 6H), 2.17 (s, 3H). |
| 36 | 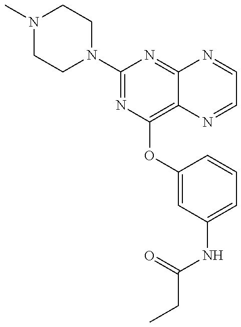 [SMILES: CCC(=O)Nc1cccc(Oc2nc(N3CCN(C)CC3)nc3nccnc23)c1] | LCMS (ES+) 394.3 (M + H)+, RT 2.36 min (Analytical method AcHSSC18) ¹H NMR (400 MHz, DMSO) δ 8.83 (d, J = 1.9 Hz, 1H), 8.52 (d, J = 1.9 Hz, 1H), 7.71 (s, 1H), 7.38 (t, J = 8.0 Hz, 1H), 7.33 (d, J = 8.4 Hz, 1H), 7.22 (s, 1H), 7.03 (d, J = 7.5 Hz, 1H), 4.08 (s, 2H), 3.67 (s, 2H), 2.78 (s, 2H), 2.58- 2.50 (m, 2H), 2.42 (q, J = 7.5 Hz, 2H), 2.34 (s, 3H), 1.25 (dd, J = 7.5, 7.5 Hz, 3H). |

### Biological Assays

The following methods are for evaluating the in vitro biology properties of the test articles.

ATP binding and cross-linking assay: Enzyme human MutSbeta (Msh2(1-934), Gene ID NP_000242.1, Msh3(1-1137), Gene ID J04810.1) was adjusted to 0.2 μM and, in experiments in the presence of 22 bp biotin-labeled DNA containing a GT-mismatch (purchased from Curia Global, TOP 5′-TCATCGATCCGCAGGCTTCAGATGG-3′; BOT 3′-AGTAGCTAGGC - - - CGAAGTCTACC-5′-Biotin), pre-incubated with DNA at equimolar concentration for indicated time and temperature. All reagents were kept on ice. Enzyme was then incubated on ice for the indicated time with 5 nM γ-[³²P]-ATP (Hartmann Analytic, FP-301) or α-[³²P]-ATP (Perkin Elmer, NEG003H250UCSBF4 or Hartmann Analytic, FP-207) and addition of cold ATP (10 μM) or ADP (0.3 μM). The total volume was 20 μL and the samples were handled in Eppendorf reaction tubes. The assay buffer was 25 mM HEPES pH=7.5, 150 mM KCl, 5 mM MgCl₂, 5 mM GSH, 0.01% Tween-20. All steps with radioactive ATP were conducted behind appropriate shielding. The samples were then placed on dry ice until they were frozen solid. They were subsequently inserted into cavities on an ice block which had been frozen overnight at −20° C., and they were irradiated with UV light (Herolab, Type: NU-15, UV Hand Lamp, 254 nm+365 nm, 15 Watt Tube, Cat. No. H469.1, Serial-Nr.: 1519003H469.1) at a distance of 10 cm for 60 minutes. Samples were then mixed with Bolt sample loading buffer (ThermoFisher) and heated to 100% for 10 minutes. A total of 10 μL were loaded to an SDS PAGE (Bolt™ 4-12% Bis-Tris Plus Gel, ThermoFisher) and electrophoresis was conducted at 120 V for 120 min. The gel was then washed in de-ionized water, stained with EZBlue™ (Brilliant Blue G-250; Sigma) and de-stained in de-ionized water. An image of the gel was obtained. The gel was bathed in Gel-Dry™ Drying Solution (ThermoFisher) for 20 min, and then placed between cellophane sheets which were pre-soaked in the same solution. The construct was then fixed into a drying frame to dry for at least 12 hours, and then placed in an exposure cassette (GE Healthcare) on a phosphor screen (Storage Phosphor Screen BAS IP MS 2025E, VWR 28-9564-75) together with a ¹⁴C standard (ARC, 0146R Carbon-14 Standards, Batch: C141006; lot #190109, Cal date Oct. 6, 2014) for a fixed time. The screen was analyzed in the imager (GE Healthcare, Fluorescent Image Analyzer, Typhoon FLA 7000, #5622514B), and the image was analyzed with imageJ software. Data for compounds disclosed herein are shown in Table 3.

TABLE 3

| Compound | cross-link assay IC₅₀ |
|---|---|
| 2 | MSH2: 120 uM/MSH3: 5 uM |
| 4 | MSH2: 215 uM/MSH3: 9 uM |

P33 SPA: Enzyme human MutSbeta (Msh2(1-934), Gene ID NP_000242.1, Msh3(1-1137), Gene ID J04810.1) and 22 bp biotin-labeled DNA containing a GT-mismatch (purchased from Curia Global, TOP 5′-TCATCGATCCGCAGGCTTCAGATGG-3′; BOT 3′-AGTAGCTAGGC - - - CGAAGTCTACC-5′-Biotin) were pre-incubated at equimolar concentration for 15 min at room temperature (“RT”) in assay buffer (25 mM HEPES pH=7.5, 150 mM KCl, 2 mM MgCl₂, 2 mM GSH, 0.01% Tween-20). ATP (10 μL, 2× final concentration of γ-[³³P]-ATP (final 0.1 nM; Hartmann Analytic, FF-301) and cold ATP (final 2 μM)) was added to compounds (400 nL spotted as concentration response in 100% DMSO into wells of an OptiPlate) and pre-incubated for 30 min at RT. Then, enzyme with DNA was added (10 μL, 2× final concentration of 7 nM) and incubated for 60 min at 37° C. in a total volume of 20 μL per well. Decay of [³³P] with a half-life of 25.4 days was corrected by increasing the concentration of the labelled nucleotide with the appropriate factor; all steps with radioactive ATP were conducted behind appropriate shielding. Then, 20 μL of a 1:2 mix of ammonium molybdate (20 mg/mL Ammonium molybdate in 2.4 M HCl) and SPA beads (ProteinA SPA beads from Perkin Elmer Cat #RPNQ0019 at 7.5 mg/mL+1% azide) was added per well and incubated at RT for 2 min. Then, 40 μL of 7 M CsCl and 0.1 M citrate was added per well and incubated for 20 h at RT before measuring on the MicroBeta (top read mode, 1 min per well). For full plate processing, all reagents were transferred with a pipettor (Selma, CyBio) apart from the final reagent (CsCl+citrate) which was added with a dispenser (MultiFlo, BioTek). Data analysis during assay development was done with Prism GraphPad.

Data for compounds disclosed herein are shown in Table 4.

TABLE 4

| Compound | P33 SPA IC₅₀ (μM) |
|---|---|
| 2 | 28.6 |
| 3 | 20.8 |
| 4 | 23.8 |
| 5 | 31 |
| 6 | 26.2 |
| 16 | 121.7 |
| 20 | 12.9 |

P33 SPA 11: For the SPA 1.1 assay, the addition of reagents was reversed, i.e. enzyme was first added to the compounds and pre-incubated followed by ATP addition; thus, the assay was aligned with ADP Glo 1.1 reagent addition order. Data for compounds disclosed herein are shown in Table 5A. The assay was repeated under similar conditions, and the results are shown below in Table 5B. It is contemplated the solubility of the compounds and/or varying test occasions may account for variability of assay results.

TABLE 5A

| Compound | P33 SPA 1.1 IC₅₀ (μM) |
|---|---|
| 2 | 55.9 |
| 3 | 24.5 |
| 4 | 40.5 |
| 5 | 44.8 |
| 6 | 41.5 |
| 16 | >200 |
| 20 | 43.4 |
| 23 | 71 |
| 24 | 24 |
| 25 | 20 |
| 26 | 59 |
| 27 | 28 |
| 28 | 55 |
| 29 | 120 |
| 30 | 23 |
| 31 | 25 |
| 32 | 118 |
| 33 | 44 |
| 34 | 43 |
| 35 | 80 |
| 36 | 66 |
| 37 | 34 |
| 38 | 19 |
| 39 | 158 |
| 40 | 43 |
| 45 | 38 |
| 46 | 104 |
| 47 | 59 |
| 48 | 12 |

TABLE 5B

| Compound | P33 SPA 1.1 IC₅₀ (μM) |
|---|---|
| 23 | 64 |
| 24 | 25 |
| 25 | 24 |
| 26 | 75 |
| 27 | 18 |
| 28 | 131 |
| 29 | 61 |
| 30 | 28 |
| 31 | 45 |
| 32 | 47 |
| 33 | 34 |
| 34 | 90 |
| 35 | 55 |
| 36 | 156 |
| 37 | 119 |
| 38 | 9.5 |
| 39 | 36 |
| 40 | 61 |
| 41 | 50 |
| 42 | 108 |
| 43 | 59 |
| 44 | 71 |
| 45 | 84 |
| 46 | 120 |
| 47 | 56 |
| 48 | 9.0 |

P33 SPA counter assay: This assay was used in the absence of enzyme to determine whether the test compound interferes with the detection system. Otherwise, the same protocol and assay buffer as in the main SPA assay were applied (25 mM HEPES pH=7.5, 150 mM KCl, 2 mM MgCl₂, 2 mM GSH, 0.01% Tween-20) and instead of enzyme addition, assay buffer was added. Hydrolysis of substrate (γ-[³³P]-ATP, 0.1 nM; Hartmann Analytic, FF-301; and cold ATP, 10 μM) to a similar level as under enzymatic conditions was achieved by incubation at 50° C. for 4 days. Decay of [³³P] with a half-life of 25.4 days was corrected by increasing the concentration of the labelled nucleotide with the appropriate factor; all steps with radioactive ATP were conducted behind appropriate shielding. After addition of pre-treated ATP substrate (10 μL) to the compounds and addition of buffer (10 μL, instead of enzyme), 20 μL of a 1:2 mix of ammonium molybdate (20 mg/mL Ammonium molybdate in 2.4M HCl) and SPA beads (ProteinA SPA beads from Perkin Elmer Cat #RPNQ0019 at 7.5 mg/mL+1% azide) was added per well and incubated at RT for 2 min. Then, 40 μL of 7 M CsCl and 0.1 M citrate was added per well and incubated for 1 h at room temperature before measuring on the MicroBeta (top read mode, 1 min per well). For full plate processing, all reagents were transferred with a pipettor (Selma, CyBio) apart from the final reagent (CsCl+citrate) which was added with a dispenser (MultiFlo, BioTek). Data analysis during assay development was done with Prism GraphPad, and IC₅₀ fitting for whole compound plates was done with the in-house software Aplus. Data for compounds disclosed herein are shown in Table 6.

TABLE 6

| Compound | P33 SPA counter assay IC₅₀ (μM) |
|---|---|
| 2 | >200 |
| 3 | >200 |
| 4 | >200 |
| 5 | >200 |
| 6 | >200 |
| 16 | >200 |
| 20 | >200 |

ADP Glo assay 2.1: Enzyme human MutSbeta (Msh2(1-934), Gene ID NP_000242.1, Msh3(1-1137), Gene ID J04810.1) with 22 bp biotin-labeled DNA containing a GT-mismatch (purchased from Curia Global, TOP 5′-TCATCGATCCGCAGGCTTCAGATGG-3′; BOT 3′-AGTAGCTAGGC - - - CGAAGTCTACC-5′-Biotin) was pre-incubated in assay buffer (25 mM HEPES pH=7.5, 150 mM KCl, 5 mM MgCl₂, 5 mM GSH, 0.1% Pluronic F-127, 0.2% Ovalbumin) to allow formation of the enzyme-DNA complex. Plates with spotted compounds in serial dilutions in DMSO (80 nL) were filled with ATP (2 μL, 10 μM final concentration) followed by an incubation of 30 min at RT. Then, enzyme was added (2 μL, 7 nM final concentration) and the reaction was allowed to proceed for 60 min at 37° C. After the ATPase reaction, the commercially available “ADP-Glo Reagent” was added (incubate 40 min at RT) to deplete remaining ATP via the activity of Adenylate cyclase and Pyrophosphatase that convert ATP into cAMP and phosphate. Subsequently, the commercially available “Kinase Detection Reagent” converted ADP to ATP via the activity of PEP and Pyruvate kinase. The ATP was then converted into light by Ultra-Glo Luciferase. The obtained luminescence was proportional to the ADP concentration generated during the initial ATPase reaction. The luminescence signal was measured with an Envision reader, raw data were analyzed towards controls (full reaction=0% inhibition; no reaction in absence of enzyme=100% inhibition) and IC₅₀ curves were analyzed with the inhouse software Aplus. Data for compounds disclosed herein are shown in Table 7A.

TABLE 7A

| Compound | ADP Glo assay 2.1 IC₅₀ (μM) |
|---|---|
| 2 | 67.3 |
| 3 | 32.6 |
| 4 | 152.3 |
| 5 | 148.1 |
| 6 | 47.1 |
| 16 | >200 |
| 20 | 139.6 |
| 23 | 200 |
| 24 | 37 |
| 25 | 29 |
| 26 | 36 |
| 27 | 22 |
| 28 | 38 |
| 29 | 200 |
| 30 | 23 |
| 31 | 19 |
| 32 | 200 |
| 33 | 35 |
| 34 | 200 |
| 35 | 200 |
| 36 | 200 |
| 37 | 71 |
| 38 | 13 |
| 39 | 200 |
| 40 | 16 |
| 41 | 28 |
| 42 | 200 |
| 45 | 55 |
| 46 | 38 |
| 47 | 55 |
| 48 | 13 |

ADP Glo assay 2.2: A similar protocol was used as in the ADP Glo Assay 2.1 above, except the following assay buffer was used: 25 mM HEPES pH=7.5, 150 mM KCl, 5 mM MgCl₂, 2 mM GSH, 0.1% Pluronic F-127, 0.2% Ovalbumin. Data for the compounds disclosed herein are shown in Table 7B. It is contemplated the solubility of the compounds and/or varying test occasions and/or assay conditions may account for variability of assay results.

TABLE 7B

| Compound | ADP Glo assay 2.2 IC₅₀ (μM) |
|---|---|
| 24 | 60 |
| 25 | 35 |
| 26 | 95 |
| 27 | 26 |
| 28 | 82 |
| 30 | 25 |
| 31 | 41 |
| 32 | 200 |
| 33 | 43 |
| 37 | 97 |
| 38 | 10 |
| 39 | 200 |
| 40 | 34 |
| 41 | 50 |
| 42 | 119 |
| 43 | 106 |
| 44 | 88 |
| 45 | 76 |
| 46 | 53 |
| 47 | 38 |
| 48 | 27 |

ADP Glo assay 1.1: The ADP Glo 2.1 assay was adapted to enable assessment of a more direct interaction of the compound with the enzyme: (1) the SPA buffer was applied (25 mM HEPES pH=7.5, 150 mM KCl, 2 mM MgCl₂, 2 mM GSH, 0.01% Tween-20) which contained no decoy Ovalbumin protein. (2) The order of reagent addition was changed, and enzyme was now added directly to the spotted compounds before addition of ATP. Data for compounds disclosed herein are shown in Table 8.

TABLE 8

| Compound | ADP Gol 1.1 assay IC₅₀ (μM) |
|---|---|
| 2 | 54.8 |
| 3 | 42.5 |
| 4 | 64.3 |
| 5 | 63.4 |
| 6 | 82.1 |
| 16 | >200 |
| 20 | 109.4 |

ADP Glo assay with MutSα (2.1): The ADP Glo assay version 2.1 with the enzyme human MutSα (Msh2(1-934), Gene ID NP_000242.1, Msh6(1-1360), Gene ID NP_001393725.1) was based on the same principle and protocol steps as the MutSβ assay. A 22 bp biotin-labeled DNA containing a GT-mismatch (purchased from Curia Global, TOP 5′-TCATCGATCCGCAGGCTTCAGATGG-3′; BOT 3′-AGTAGCTAGGC - - - CGAAGTCTACC-5′-Biotin) was used for binding to MutSα. For this assay, concentrations of enzyme and ATP were optimized to 15 nM MutSα/DNA and 30 μM ATP. Data for compounds disclosed herein are shown in Table 9.

TABLE 9

| | ADP Glo assay with MutSα |
|---|---|
| Compound | (2.1) IC₅₀ (μM) |
| 2 | 135 |
| 3 | 42.3 |
| 4 | 121 |
| 5 | >200 |
| 6 | — |
| 16 | 42.7 |
| 20 | 26.9 |

Unless otherwise defined, all technical and scientific terms used herein have the same meaning as commonly understood by one of ordinary skill in the art to which this disclosure belongs.

The disclosure illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claims.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A compound of Formula I: 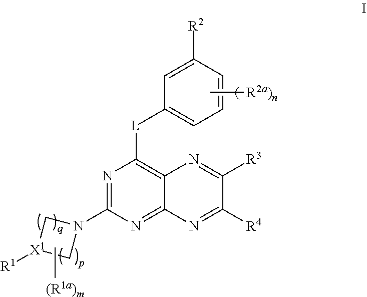
or a pharmaceutically acceptable salt, isotopically enriched analog, stereoisomer, mixture of stereoisomers, or prodrug thereof, wherein:
n is 0, 1, 2, 3, or 4;
m is 0, 1, 2, 3, 4, or 5;
p is 0, 1, 2, 3, 4, or 5;
q is 0, 1, 2, 3, or 4; provided that p+q is 2, 3, 4, or 5;
L is —NH—, —O—, —CH₂—, —NHCH₂—, —OCH₂—, or —CH₂CH₂—;
X¹ is CR⁵ or N;
R¹ is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R^{1a} and R^{2a} are each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R² is halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R³ and R⁴ are each independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —O—R¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R⁵ is hydrogen, C₁₋₆ alkyl, or —N(R¹¹)₂;
each R¹⁰ is independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —OR¹¹, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl; and
each R¹¹ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl;
provided that:
when L is —NH—, R³ and R⁴ are both hydrogen, and the moiety 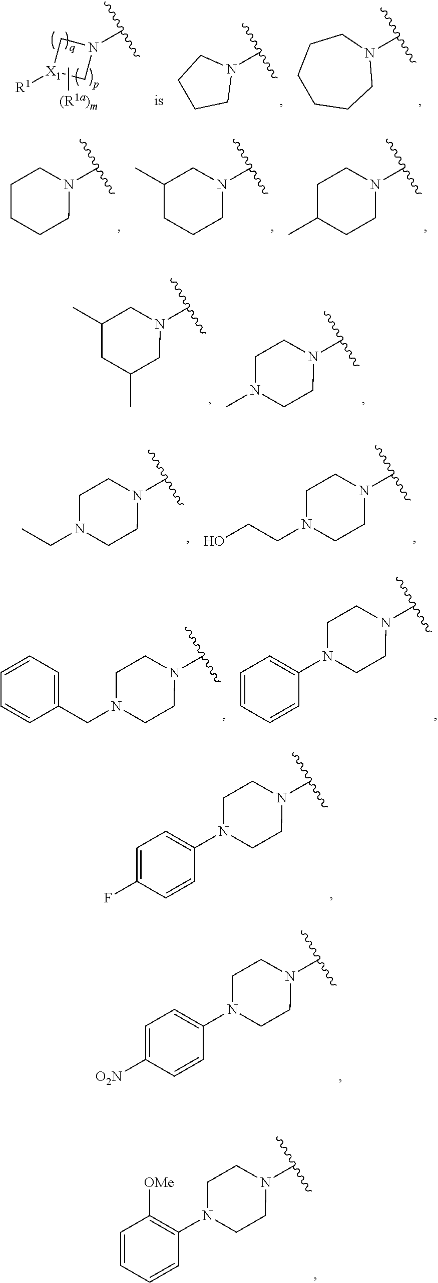 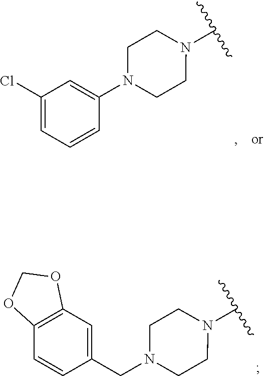 [SMILES: *N1CCN(c2cccc(Cl)c2)CC1 ; *N1CCN(Cc2ccc3c(c2)OCO3)CC1]
then the moiety

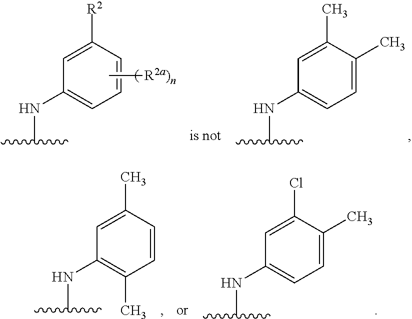

2. The compound of claim 1, wherein p+q is 3 or 4.

3. The compound of claim 1, wherein p is 2 and q is 2.

4. The compound of claim 1, wherein the compound is represented by Formula IA: 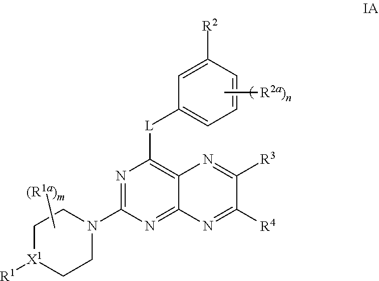
or a pharmaceutically acceptable salt, isotopically enriched analog, stereoisomer, mixture of stereoisomers, or prodrug thereof.

5. The compound of claim 1, wherein X¹ is N.

6. The compound of claim 1, wherein R¹ is C₁₋₆ alkyl optionally substituted with 1-5 R¹⁰.

7.-8. (canceled)

9. The compound of claim 1, wherein the compound is represented by Formula IB: 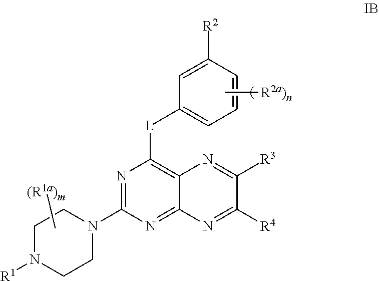
or a pharmaceutically acceptable salt, isotopically enriched analog, stereoisomer, mixture of stereoisomers, or prodrug thereof.

10. The compound of claim 1, wherein L is —NH—.

11. The compound of claim 1, wherein the compound is represented by Formula IC: 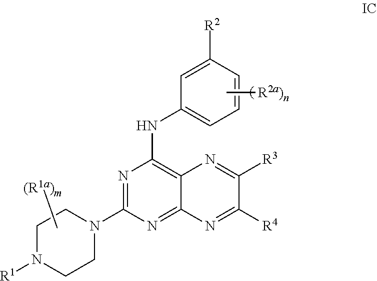
or a pharmaceutically acceptable salt, isotopically enriched analog, stereoisomer, mixture of stereoisomers, or prodrug thereof.

12. The compound of claim 1, wherein n is 0 or 1.

13. The compound of claim 1, wherein the compound is represented by Formula ID: 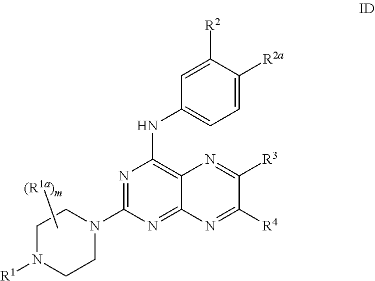
or a pharmaceutically acceptable salt, isotopically enriched analog, stereoisomer, mixture of stereoisomers, or prodrug thereof.

14. The compound of claim 1, wherein the compound is represented by Formula IE: 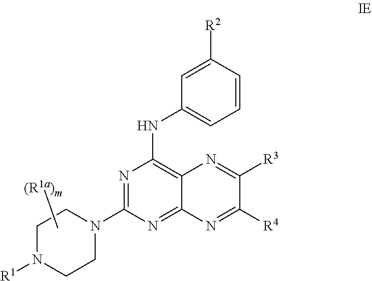
or a pharmaceutically acceptable salt, isotopically enriched analog, stereoisomer, mixture of stereoisomers, or prodrug thereof.

15. The compound of claim 1, wherein R² and R^{2a} are each independently halo or C₁₋₆ alkyl.

16. (canceled)

17. The compound of claim 13, wherein the moiety 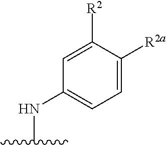
is:

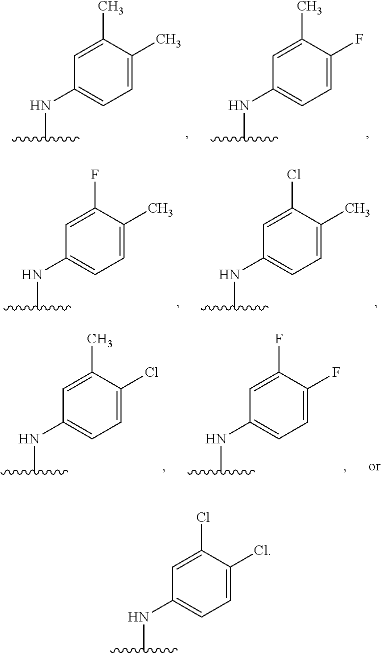

18. The compound of claim 14, wherein the moiety 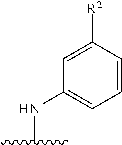
is:

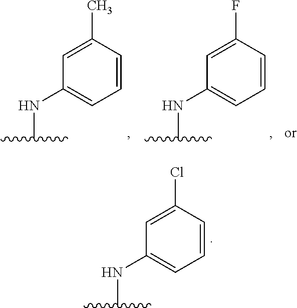

19. The compound of claim 1, wherein each R³ and R⁴ is independently hydrogen, halo, or cyano.

20. The compound of claim 1, wherein R³ and R⁴ are each independently hydrogen or C₁₋₃ alkyl.

21. (canceled)

22. The compound of claim 1, wherein m is 0.

23. The compound of claim 1, wherein the compound is represented by Formula IG: 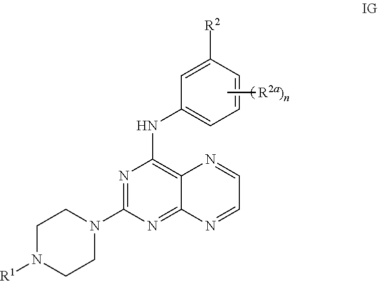
or a pharmaceutically acceptable salt, isotopically enriched analog, stereoisomer, mixture of stereoisomers, or prodrug thereof.

24. The compound of claim 1, wherein:
the moiety 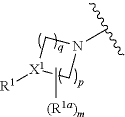
is 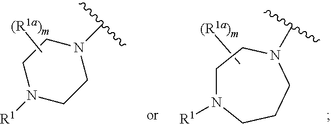
n is 0 or 1;
m is 0 or 1;
L is —NH—, —O—, or —NHCH₂—;
R¹ is C₁₋₃ alkyl; and
R³ and R⁴ are each independently hydrogen or C₁₋₃ alkyl.

25. The compound of claim 1, wherein:
the moiety 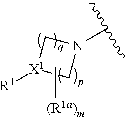
is 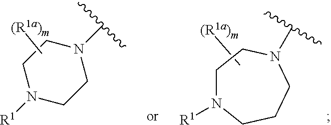
n is 0 or 1;
m is 0 or 1;
L is —NH—, —O—, or —NHCH₂—;
R¹ is C₁₋₃ alkyl;
R^{1a} is C₁₋₃ alkyl; and
R³ and R⁴ are each independently hydrogen or C₁₋₃ alkyl.

26. The compound of claim 1, wherein the moiety

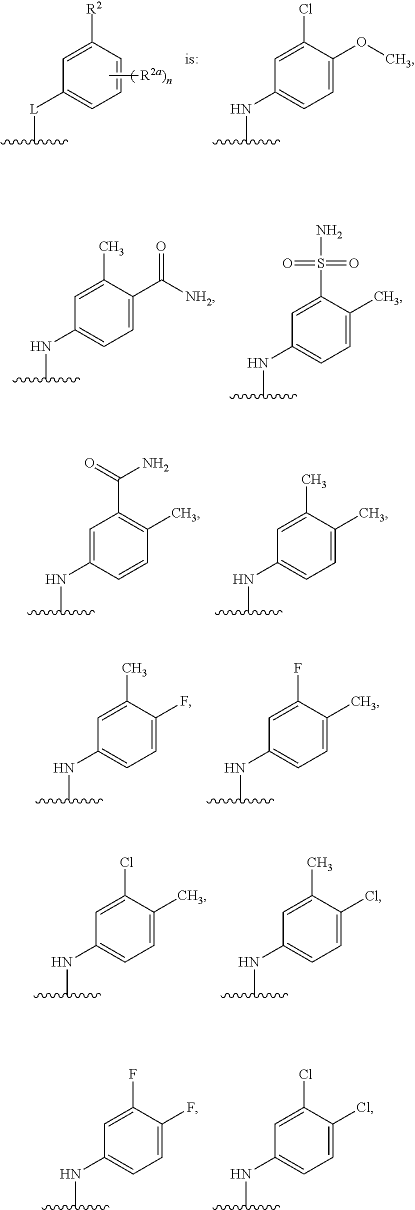

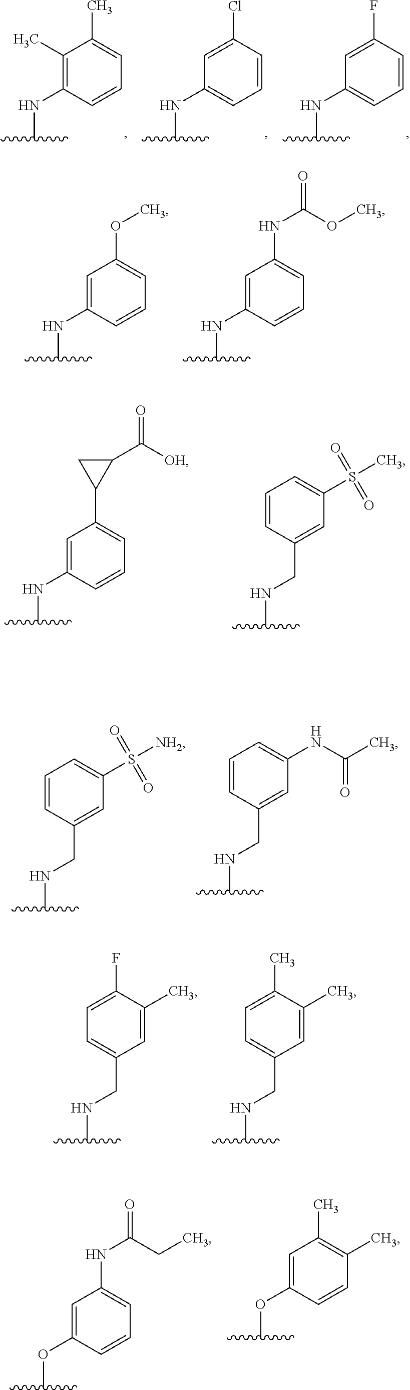

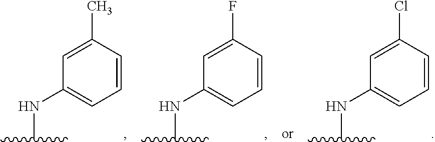

27. The compound of claim 1, or a pharmaceutically acceptable salt, isotopically enriched analog, stereoisomer, mixture of stereoisomers, or prodrug thereof, wherein the compound is selected from:

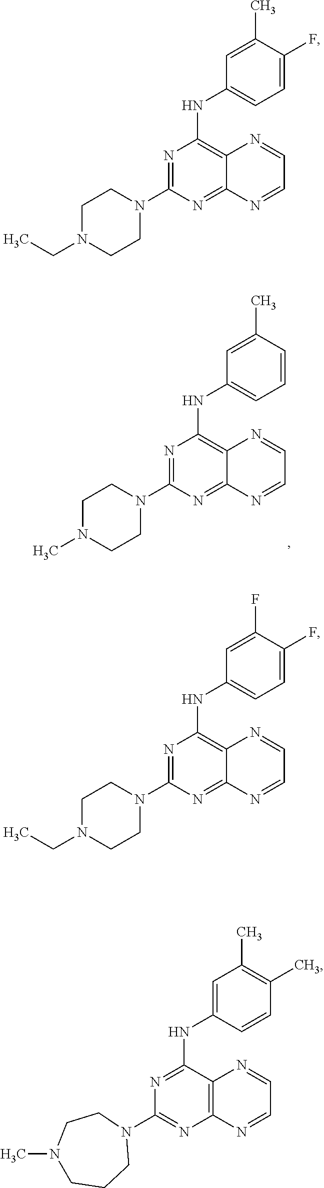 [SMILES: CCN1CCN(c2nc(Nc3ccc(F)c(C)c3)c3nccnc3n2)CC1 ; Cc1cccc(Nc2nc(N3CCN(C)CC3)nc3nccnc23)c1 ; CCN1CCN(c2nc(Nc3ccc(F)c(F)c3)c3nccnc3n2)CC1 ; Cc1ccc(Nc2nc(N3CCCN(C)CC3)nc3nccnc23)cc1C]

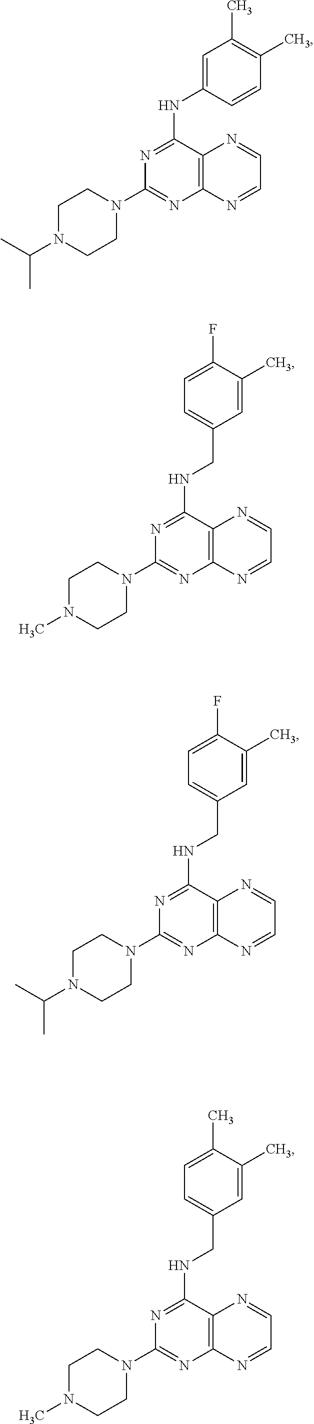 [SMILES: Cc1ccc(Nc2nc(N3CCN(C(C)C)CC3)nc3nccnc23)cc1C ; Cc1cc(CNc2nc(N3CCN(C)CC3)nc3nccnc23)ccc1F ; Cc1cc(CNc2nc(N3CCN(C(C)C)CC3)nc3nccnc23)ccc1F ; Cc1ccc(CNc2nc(N3CCN(C)CC3)nc3nccnc23)cc1C]

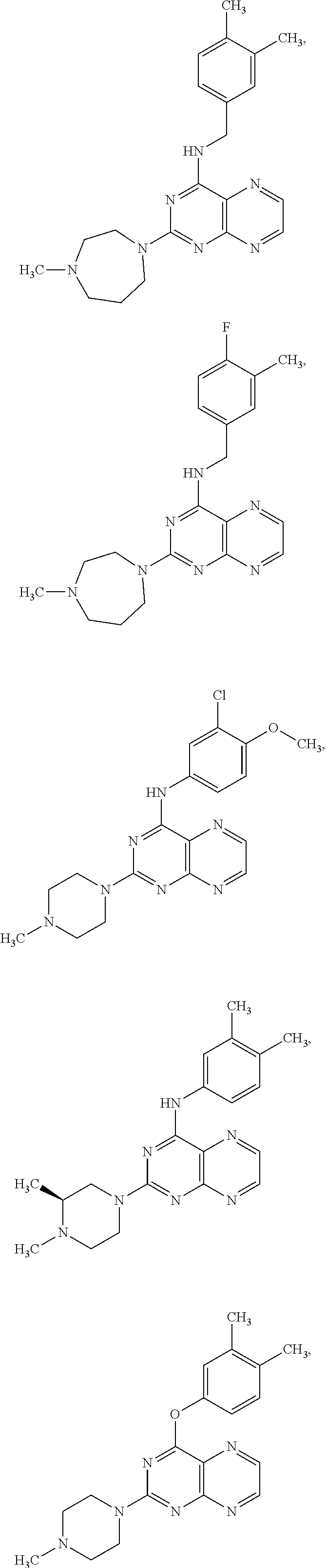 [SMILES: Cc1ccc(CNc2nc(N3CCCN(C)CC3)nc3nccnc23)cc1C ; Cc1cc(CNc2nc(N3CCCN(C)CC3)nc3nccnc23)ccc1F ; COc1ccc(Nc2nc(N3CCN(C)CC3)nc3nccnc23)cc1Cl ; Cc1ccc(Nc2nc(N3CCN(C)[C@@H](C)C3)nc3nccnc23)cc1C ; Cc1ccc(Oc2nc(N3CCN(C)CC3)nc3nccnc23)cc1C]

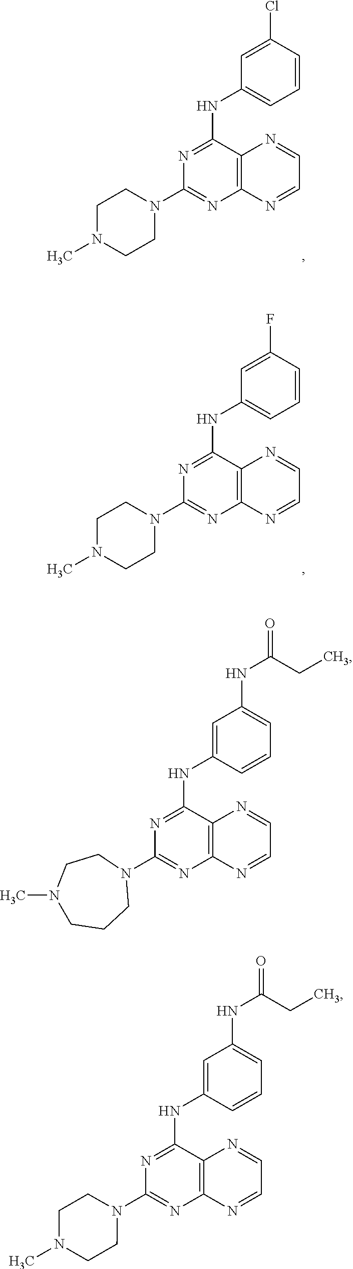 [SMILES: CN1CCN(c2nc(Nc3cccc(Cl)c3)c3nccnc3n2)CC1 ; CN1CCN(c2nc(Nc3cccc(F)c3)c3nccnc3n2)CC1 ; CCC(=O)Nc1cccc(Nc2nc(N3CCCN(C)CC3)nc3nccnc23)c1 ; CCC(=O)Nc1cccc(Nc2nc(N3CCN(C)CC3)nc3nccnc23)c1]

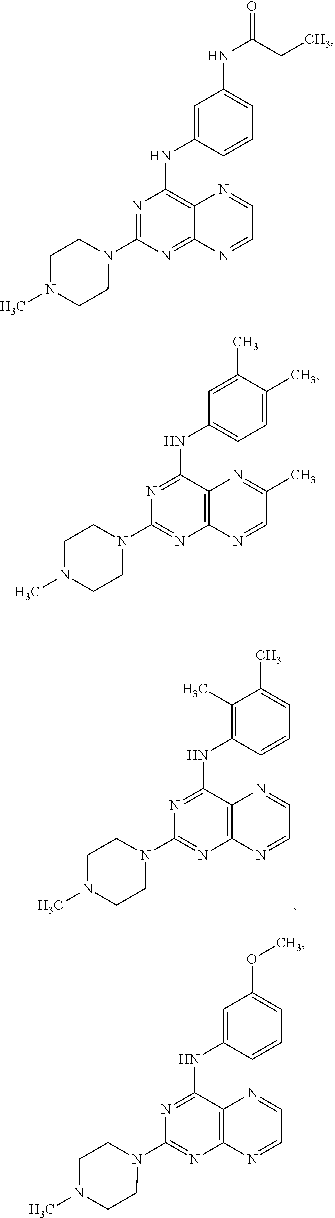

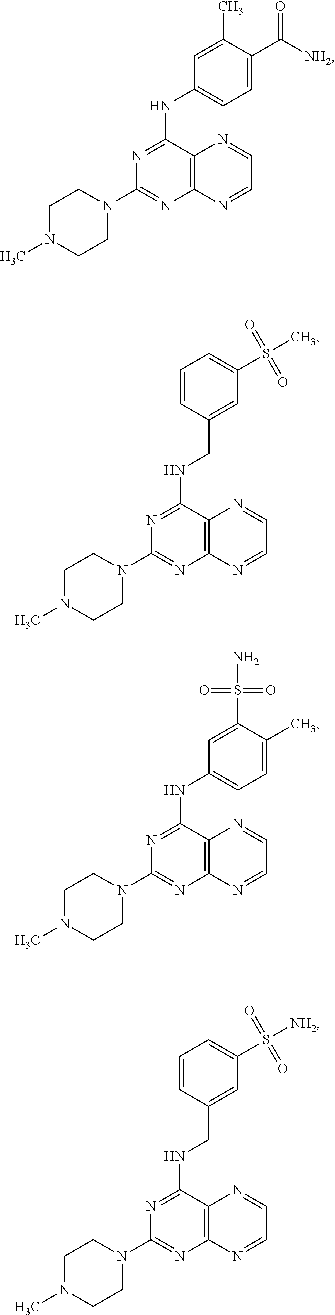 [SMILES: Cc1cc(Nc2nc(N3CCN(C)CC3)nc3nccnc23)ccc1C(N)=O ; CN1CCN(c2nc(NCc3cccc(S(C)(=O)=O)c3)c3nccnc3n2)CC1 ; Cc1ccc(Nc2nc(N3CCN(C)CC3)nc3nccnc23)cc1S(N)(=O)=O ; CN1CCN(c2nc(NCc3cccc(S(N)(=O)=O)c3)c3nccnc3n2)CC1]

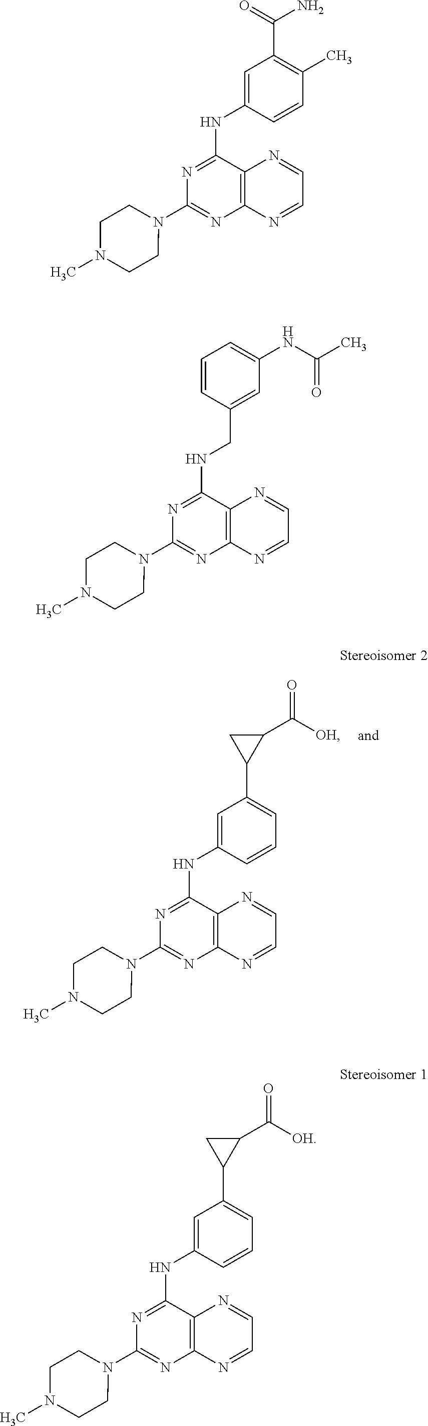

28. A pharmaceutical composition comprising the compound of claim 1, or a pharmaceutically acceptable salt, isotopically enriched analog, stereoisomer, mixture of stereoisomers, or prodrug thereof, and a pharmaceutically acceptable excipient.

29. A method for treating a disease or disorder modulated, at least in part, by MutSβ, in a subject in need thereof, the method comprising administering to the subject an effective amount of a compound of Formula I: 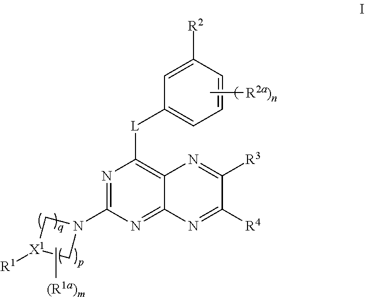
or a pharmaceutically acceptable salt, isotopically enriched analog, stereoisomer, mixture of stereoisomers, or prodrug thereof, wherein:
n is 0, 1, 2, 3, or 4;
m is 0, 1, 2, 3, 4, or 5;
p is 0, 1, 2, 3, 4, or 5;
q is 0, 1, 2, 3, or 4; provided that p+q is 2, 3, 4, or 5;
L is —NH—, —O—, —CH₂—, —NHCH₂—, —OCH₂—, or —CH₂CH₂—;
X¹ is O, N, or CH; provided that when X¹ is O, then R¹ is absent;
R¹, when present, is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R^{1a} and R^{2a} are each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R² is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R³ and R⁴ are each independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
each R¹⁰ is independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —OR¹¹, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl; and
each R¹¹ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl.

30. A method for inhibiting the activity of MutSβ, the method comprising administering to a subject in need thereof, an effective amount of a compound of Formula I: 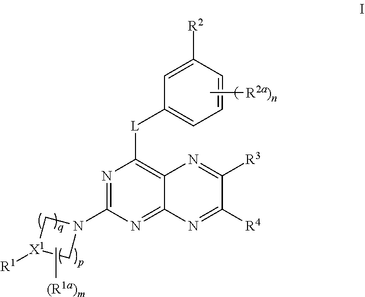
or a pharmaceutically acceptable salt, isotopically enriched analog, stereoisomer, mixture of stereoisomers, or prodrug thereof, wherein:
n is 0, 1, 2, 3, or 4;
m is 0, 1, 2, 3, 4, or 5;
p is 0, 1, 2, 3, 4, or 5;
q is 0, 1, 2, 3, or 4; provided that p+q is 2, 3, 4, or 5;
L is —NH—, —O—, —CH₂—, —NHCH₂—, —OCH₂—, or —CH₂CH₂—;
X¹ is O, N, or CH; provided that when X¹ is O, then R¹ is absent;
R¹, when present, is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R^{1a} and R^{2a} are each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R² is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R³ and R⁴ are each independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
each R¹⁰ is independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —OR¹¹, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl; and
each R¹¹ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl.

31. A method for treating a neurodegenerative or neurological disease or disorder, the method comprising administering to a subject in need thereof, an effective amount of a compound of Formula I: 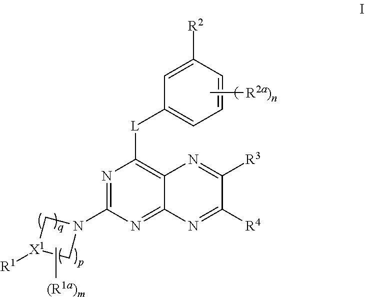
or a pharmaceutically acceptable salt, isotopically enriched analog, stereoisomer, mixture of stereoisomers, or prodrug thereof, wherein:
n is 0, 1, 2, 3, or 4;
m is 0, 1, 2, 3, 4, or 5;
p is 0, 1, 2, 3, 4, or 5;
q is 0, 1, 2, 3, or 4; provided that p+q is 2, 3, 4, or 5;
L is —NH—, —O—, —CH₂—, —NHCH₂—, —OCH₂—, or —CH₂CH₂—;
X¹ is O, N, or CH; provided that when X¹ is O, then R¹ is absent;
R¹, when present, is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R^{1a} and R^{2a} are each independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R² is hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
R³ and R⁴ are each independently hydrogen, halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, or heteroaryl is independently optionally substituted with 1-5 R¹⁰;
each R¹⁰ is independently halo, cyano, C₁₋₆ alkyl, C₂₋₆ alkenyl, C₂₋₆ alkynyl, C₁₋₆ haloalkyl, C₃₋₁₀ cycloalkyl, C₃₋₁₀ cycloalkenyl, heterocyclyl, aryl, heteroaryl, —OR¹¹, —N(R¹¹)₂, —C(O)R¹¹, —C(O)OR¹¹, —S—R¹¹, S(O)R¹¹, —S(O)₂R¹¹, —NR¹¹S(O)R¹¹, —NR¹¹S(O)₂R¹¹, —S(O)N(R¹¹)₂, —S(O)₂N(R¹¹)₂, —NR¹¹S(O)N(R¹¹)₂, —NR¹¹S(O)₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, C₃₋₁₀ cycloalkenyl, aryl, heteroaryl, or heterocyclyl; and
each R¹¹ is independently hydrogen, C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₁₋₆ heteroalkyl, C₃₋₁₀ cycloalkyl, heterocyclyl, aryl, or heteroaryl is independently optionally substituted with 1-5 halo, cyano, —NO₂, oxo, C₁₋₆ alkyl, C₁₋₆ haloalkyl, C₁₋₆ alkoxy, C₁₋₆ haloalkoxy, C₃₋₁₀ cycloalkyl, aryl, heteroaryl, or heterocyclyl.

32. The method of claim 29, wherein the compound, or pharmaceutically acceptable salt, isotopically enriched analog, stereoisomer, mixture of stereoisomers, or prodrug thereof, is selected from Table 1.

33. The method of claim 31, wherein the neurodegenerative or neurological disease or disorder is Huntington's disease (HD), spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 8, spinocerebellar ataxia type 12, spinocerebellar ataxia type 17, Huntington's disease-like 2, fragile X syndrome, fragile X-associated tremor/ataxia syndrome, fragile XE mental retardation, Friedreich's ataxia, myotonic dystrophy type 1, oculopharyngeal muscular dystrophy, or dentatorubro-pallidoluysian atrophy.

34. The method of claim 31, wherein the neurodegenerative or neurological disease or disorder is Huntington's Disease (HD).

35.-36. (canceled)
